SEQUENCE LISTING (250 sequences)

SEQ ID NO: 1 - protein, 124 aa, synthetic construct
  source 1..124: note = 70416_041[TL1a]_H1L1 Variable Heavy (vh) Domain
QVQLVQSGAEVKKPGASVKVSCKVSGFSVTSYNVHWVRQAPGQGLEWMGVMWSGGNTDYNSKFQGRVTMTRDTSTNTVYMELSSLRSEDTAVYYCARAGRYYHDSNYYWDFPYWGQGTLVTVSS

SEQ ID NO: 2 - protein, 5 aa, synthetic construct
  source 1..5: note = 70416_041[TL1a]_H1L1 vhCDR1
SYNVH

SEQ ID NO: 3 - protein, 16 aa, synthetic construct
  source 1..16: note = 70416_041[TL1a]_H1L1 vhCDR2
VMWSGGNTDYNSKFQG

SEQ ID NO: 4 - protein, 16 aa, synthetic construct
  source 1..16: note = 70416_041[TL1a]_H1L1 vhCDR3
AGRYYHDSNYYWDFPY

SEQ ID NO: 5 - protein, 107 aa, synthetic construct
  source 1..107: note = 70416_041[TL1a]_H1L1 Variable Light (vl) Domain
DIQMTQSPSSVSASVGDRVTITCRASEDIYSGLAWYQQKPGKSPKLLIYGASSLQDGVPSRFSGSGSGTDYTLTISSLQPEDFATYFCQEGLKSPYTFGAGTKLEIK

SEQ ID NO: 6 - protein, 11 aa, synthetic construct
  source 1..11: note = 70416_041[TL1a]_H1L1 vlCDR1
RASEDIYSGLA

SEQ ID NO: 7 - protein, 7 aa, synthetic construct
  source 1..7: note = 70416_041[TL1a]_H1L1 vlCDR2
GASSLQD

SEQ ID NO: 8 - protein, 9 aa, synthetic construct
  source 1..9: note = 70416_041[TL1a]_H1L1 vlCDR3
QEGLKSPYT

SEQ ID NO: 9 - protein, 119 aa, synthetic construct
  source 1..119: note = 70417_111 [TL1a]_H1L1 Variable Heavy (vh) Domain
EVQLVESGGGLVQPGGSLRLSCAASGFTFSSFGMHWVRQAPGKGLEWVSYISSGSTTIYYADSVKGRFTISRDNAKNSLYLQMNSLRAEDTAVYYCARAPLLRGAMDYWGQGTLVTVSS

SEQ ID NO: 10 - protein, 5 aa, synthetic construct
  source 1..5: note = 70417_111 [TL1a]_H1L1 vhCDR1
SFGMH

SEQ ID NO: 11 - protein, 17 aa, synthetic construct
  source 1..17: note = 70417_111 [TL1a]_H1L1 vhCDR2
YISSGSTTIYYADSVKG

SEQ ID NO: 12 - protein, 10 aa, synthetic construct
  source 1..10: note = 70417_111 [TL1a]_H1L1 vhCDR3
APLLRGAMDY

SEQ ID NO: 13 - protein, 107 aa, synthetic construct
  source 1..107: note = 70417_111 [TL1a]_H1L1 Variable Light (vl) Domain
DIVMTQSPDSLAVSLGERATINCKASQDVSTAVVWYQQKPGQSPKLLIYWASTRHTGVPDRFTGSGSGTDYTLTISSLQAEDVAVYHCQQHYSTPYTFGGGTKLEIK

SEQ ID NO: 14 - protein, 11 aa, synthetic construct
  source 1..11: note = 70417_111 [TL1a]_H1L1 vlCDR1
KASQDVSTAVV

SEQ ID NO: 15 - protein, 7 aa, synthetic construct
  source 1..7: note = 70417_111 [TL1a]_H1L1 vlCDR2
WASTRHT

SEQ ID NO: 16 - protein, 9 aa, synthetic construct
  source 1..9: note = 70417_111 [TL1a]_H1L1 vlCDR3
QQHYSTPYT

SEQ ID NO: 17 - protein, 114 aa, synthetic construct
  source 1..114: note = 70392_069 [TL1a]_H1L1 Variable Heavy (vh) Domain
EVQLVESGGGLVQPGGSLKLSCAASGFDIIDDYIHWVRQASGKGLEWVGRIDPASANTKYVPSVKGRFTISADTSKNTAYLQMNSLKTEDTAVYYCTRKGFAYWGQGTLVTVSS

SEQ ID NO: 18 - protein, 5 aa, synthetic construct
  source 1..5: note = 70392_069 [TL1a]_H1L1 vhCDR1
DDYIH

SEQ ID NO: 19 - protein, 17 aa, synthetic construct
  source 1..17: note = 70392_069 [TL1a]_H1L1 vhCDR2
RIDPASANTKYVPSVKG

SEQ ID NO: 20 - protein, 5 aa, synthetic construct
  source 1..5: note = 70392_069 [TL1a]_H1L1 vhCDR3
KGFAY

SEQ ID NO: 21 - protein, 107 aa, synthetic construct
  source 1..107: note = 70392_069 [TL1a]_H1L1 Variable Light (vl) Domain
NIVMTQTPLSLSVTPGQPASISCKASENVGTYVSWYLQKPGQSPQLLIYGTSNRYTGVPDRFTGSGSATDFTLKISRVEAEDVGVYHCGQSYRYPYTFGGGTKLEIK

SEQ ID NO: 22 - protein, 11 aa, synthetic construct
  source 1..11: note = 70392_069 [TL1a]_H1L1 vlCDR1
KASENVGTYVS

SEQ ID NO: 23 - protein, 7 aa, synthetic construct
  source 1..7: note = 70392_069 [TL1a]_H1L1 vlCDR2
GTSNRYT

SEQ ID NO: 24 - protein, 9 aa, synthetic construct
  source 1..9: note = 70392_069 [TL1a]_H1L1 vlCDR3
GQSYRYPYT

SEQ ID NO: 25 - protein, 119 aa, synthetic construct
  source 1..119: note = 70392_189[TL1a]_H1L1 Variable Heavy (vh) Domain
QVQLVQSGAEVKKPGASVKVSCKASGYTFTDYEIHWMRQAPGQGLEWMGGIDPETGGTAYNQKFQGRVTMTADKSISTAYMELSRLRSDDTAVYYCTRFLVHHYAMDYWGQGTTVTVSS

SEQ ID NO: 26 - protein, 5 aa, synthetic construct
  source 1..5: note = 70392_189[TL1a]_H1L1 vhCDR1
DYEIH

SEQ ID NO: 27 - protein, 17 aa, synthetic construct
  source 1..17: note = 70392_189[TL1a]_H1L1 vhCDR2
GIDPETGGTAYNQKFQG

SEQ ID NO: 28 - protein, 10 aa, synthetic construct
  source 1..10: note = 70392_189[TL1a]_H1L1 vhCDR3
FLVHHYAMDY

SEQ ID NO: 29 - protein, 112 aa, synthetic construct
  source 1..112: note = 70392_189[TL1a]_H1L1 Variable Light (vl) Domain
DVVMTQSPLSLPVTLGQPASISCRSSQSLLDSDGKTYLNWLQKRPGQSPRRLIYLVSKLDSGVPDRFTGSGSGTDFTLKISRVEAEDVGVYYCWQGTQLPFTFGAGTKLEIK

SEQ ID NO: 30 - protein, 16 aa, synthetic construct
  source 1..16: note = 70392_189[TL1a]_H1L1 vlCDR1
RSSQSLLDSDGKTYLN

SEQ ID NO: 31 - protein, 7 aa, synthetic construct
  source 1..7: note = 70392_189[TL1a]_H1L1 vlCDR2
LVSKLDS

SEQ ID NO: 32 - protein, 9 aa, synthetic construct
  source 1..9: note = 70392_189[TL1a]_H1L1 vlCDR3
WQGTQLPFT

SEQ ID NO: 33 - protein, 118 aa, synthetic construct
  source 1..118: note = 70416_092 [TL1a]_H1L1 Variable Heavy (vh) Domain
QVQLVQSGAEVKKPGASVKVSCKVSGFSLTNYGVSWVRQAPGQGLEWMGGIWGDGSTDYDSKFQGRVTMTRDTSTSTVYMELSSLRSEDTAVYFCTRYSSYTNWFVYWGQGTLVTVSS

SEQ ID NO: 34 - protein, 5 aa, synthetic construct
  source 1..5: note = 70416_092 [TL1a]_H1L1 vhCDR1
NYGVS

SEQ ID NO: 35 - protein, 16 aa, synthetic construct
  source 1..16: note = 70416_092 [TL1a]_H1L1 vhCDR2
GIWGDGSTDYDSKFQG

SEQ ID NO: 36 - protein, 10 aa, synthetic construct
  source 1..10: note = 70416_092 [TL1a]_H1L1 vhCDR3
YSSYTNWFVY

SEQ ID NO: 37 - protein, 107 aa, synthetic construct
  source 1..107: note = 70416_092 [TL1a]_H1L1 Variable Light (vl) Domain
DIQMTQSPSSLSASVGDRVTITCRAREDIYNGLAWYQQKPGKSPKLLIYNANRLHTGVPSRFSGSGSGTDFTLTISSLQPEDFATYFCQQYYDYPLTFGSGTKVEIK

SEQ ID NO: 38 - protein, 11 aa, synthetic construct
  source 1..11: note = 70416_092 [TL1a]_H1L1 vlCDR1
RAREDIYNGLA

SEQ ID NO: 39 - protein, 7 aa, synthetic construct
  source 1..7: note = 70416_092 [TL1a]_H1L1 vlCDR2
NANRLHT

SEQ ID NO: 40 - protein, 9 aa, synthetic construct
  source 1..9: note = 70416_092 [TL1a]_H1L1 vlCDR3
QQYYDYPLT

SEQ ID NO: 41 - protein, 117 aa, synthetic construct
  source 1..117: note = 70392_201 [TL1a]_H1L1 Variable Heavy (vh) Domain
QVQLVQSGAEVKKPGASVKVSCKASGYTFTDYYMDWVRQAPGQSLEWMGYIYPHTGRTDYNQKFQGRVTMTVDKSISTAYMELSRLRSDDTAVYFCARDSNYYFDVWGAGTTVTVSS

SEQ ID NO: 42 - protein, 5 aa, synthetic construct
  source 1..5: note = 70392_201 [TL1a]_H1L1 vhCDR1
DYYMD

SEQ ID NO: 43 - protein, 17 aa, synthetic construct
  source 1..17: note = 70392_201 [TL1a]_H1L1 vhCDR2
YIYPHTGRTDYNQKFQG

SEQ ID NO: 44 - protein, 8 aa, synthetic construct
  source 1..8: note = 70392_201 [TL1a]_H1L1 vhCDR3
DSNYYFDV

SEQ ID NO: 45 - protein, 107 aa, synthetic construct
  source 1..107: note = 70392_201 [TL1a]_H1L1 Variable Light (vl) Domain
DIVMTQSPLSLPVTPGEPASISCRASQNVRTAVAWYLQKPGQSPQALIYLASNRHTGVPDRFTGSGSGTDFTLKISRVEAEDVGVYFCLQHWNYPYTFGGGTKLEIK

SEQ ID NO: 46 - protein, 11 aa, synthetic construct
  source 1..11: note = 70392_201 [TL1a]_H1L1 vlCDR1
RASQNVRTAVA

SEQ ID NO: 47 - protein, 7 aa, synthetic construct
  source 1..7: note = 70392_201 [TL1a]_H1L1 vlCDR2
LASNRHT

SEQ ID NO: 48 - protein, 9 aa, synthetic construct
  source 1..9: note = 70392_201 [TL1a]_H1L1 vlCDR3
LQHWNYPYT

SEQ ID NO: 49 - protein, 119 aa, synthetic construct
  source 1..119: note = 70416_073 [TL1a]_H1L1 Variable Heavy (vh) Domain
QVQLVQSGAEVKKPGASVKVSCKVSGFSLNTYGVIWVRQAPGQGLEWMGVIWANGNTNYNSKLQGRVTMTRDTSTSTVYMELRSLRSDDTAVYFCARARYYYSGVLDYWGQGTLVTVSS

SEQ ID NO: 50 - protein, 5 aa, synthetic construct
  source 1..5: note = 70416_073 [TL1a]_H1L1 vhCDR1
TYGVI

SEQ ID NO: 51 - protein, 16 aa, synthetic construct
  source 1..16: note = 70416_073 [TL1a]_H1L1 vhCDR2
VIWANGNTNYNSKLQG

SEQ ID NO: 52 - protein, 11 aa, synthetic construct
  source 1..11: note = 70416_073 [TL1a]_H1L1 vhCDR3
ARYYYSGVLDY

SEQ ID NO: 53 - protein, 112 aa, synthetic construct
  source 1..112: note = 70416_073 [TL1a]_H1L1 Variable Light (vl) Domain
DVVLTQSPLSLPVTLGQPASISCRSSQSLVHSDGNTYLDWYQQRPGQSPRLLIYKVSNRFSGVPDRFIGSGSGSDFTLKISRVEAEDVGVYYCFQATHDPWAFGGGTKVEIK

SEQ ID NO: 54 - protein, 16 aa, synthetic construct
  source 1..16: note = 70416_073 [TL1a]_H1L1 vlCDR1
RSSQSLVHSDGNTYLD

SEQ ID NO: 55 - protein, 7 aa, synthetic construct
  source 1..7: note = 70416_073 [TL1a]_H1L1 vlCDR2
KVSNRFS

SEQ ID NO: 56 - protein, 9 aa, synthetic construct
  source 1..9: note = 70416_073 [TL1a]_H1L1 vlCDR3
FQATHDPWA

SEQ ID NO: 57 - protein, 114 aa, synthetic construct
  source 1..114: note = 70392_074 [TL1a]_H1L1 Variable Heavy (vh) Domain
QVQLVQSGAEVKKPGASVKVSCKASDFNIEDDYIHWVRQAPGQGLEWMGRIEPAGGNTKYAPKFQGRVTMTADTSINTAYMELSRLRSDDTAVYYCARKGFAYWGQGTLVTVSS

SEQ ID NO: 58 - protein, 5 aa, synthetic construct
  source 1..5: note = 70392_074 [TL1a]_H1L1 vhCDR1
DDYIH

SEQ ID NO: 59 - protein, 17 aa, synthetic construct
  source 1..17: note = 70392_074 [TL1a]_H1L1 vhCDR2
RIEPAGGNTKYAPKFQG

SEQ ID NO: 60 - protein, 5 aa, synthetic construct
  source 1..5: note = 70392_074 [TL1a]_H1L1 vhCDR3
KGFAY

SEQ ID NO: 61 - protein, 107 aa, synthetic construct
  source 1..107: note = 70392_074 [TL1a]_H1L1 Variable Light (vl) Domain
KIVMTQSPDSLAVSLGERATINCKASENVGTYVSWYQQKPGQSPKLLIYGTSNRYTGVPDRFTGSGSATDFTLTISSLQAEDVAVYHCGQSYSYPYTFGGGTKLEIK

SEQ ID NO: 62 - protein, 11 aa, synthetic construct
  source 1..11: note = 70392_074 [TL1a]_H1L1 vlCDR1
KASENVGTYVS

SEQ ID NO: 63 - protein, 7 aa, synthetic construct
  source 1..7: note = 70392_074 [TL1a]_H1L1 vlCDR2
GTSNRYT

SEQ ID NO: 64 - protein, 9 aa, synthetic construct
  source 1..9: note = 70392_074 [TL1a]_H1L1 vlCDR3
GQSYSYPYT

SEQ ID NO: 65 - protein, 124 aa, synthetic construct
  source 1..124: note = 70416_094 [TL1a]_H1L1 Variable Heavy (vh) Domain
EVQLVESGGGLVKPGGSLRLSCAVSGFSLTSHSVSWVRQAPGKGPEWVGRMWYDGDTVYNSPVKGRFTISRDTSKNTLYLQMNSLKTEDTAVYYCTRNSRYYDVSYHRFVMDAWGQGTTVTVSS

SEQ ID NO: 66 - protein, 5 aa, synthetic construct
  source 1..5: note = 70416_094 [TL1a]_H1L1 vhCDR1
SHSVS

SEQ ID NO: 67 - protein, 16 aa, synthetic construct
  source 1..16: note = 70416_094 [TL1a]_H1L1 vhCDR2
RMWYDGDTVYNSPVKG

SEQ ID NO: 68 - protein, 16 aa, synthetic construct
  source 1..16: note = 70416_094 [TL1a]_H1L1 vhCDR3
NSRYYDVSYHRFVMDA

SEQ ID NO: 69 - protein, 107 aa, synthetic construct
  source 1..107: note = 70416_094 [TL1a]_H1L1 Variable Light (vl) Domain
DTVLTQSPSSLSASVGDRVTITCRASESVSTGMHWYQQKPGKQPKLLIYGASNLESGVPARFSGSGSGTDFTLTISSLQPEDFATYFCQQSWSDPWTFGGGTKVEIK

SEQ ID NO: 70 - protein, 11 aa, synthetic construct
  source 1..11: note = 70416_094 [TL1a]_H1L1 vlCDR1
RASESVSTGMH

SEQ ID NO: 71 - protein, 7 aa, synthetic construct
  source 1..7: note = 70416_094 [TL1a]_H1L1 vlCDR2
GASNLES

SEQ ID NO: 72 - protein, 9 aa, synthetic construct
  source 1..9: note = 70416_094 [TL1a]_H1L1 vlCDR3
QQSWSDPWT

SEQ ID NO: 73 - protein, 119 aa, synthetic construct
  source 1..119: note = 70392_292 [TL1a]_H1L1 Variable Heavy (vh) Domain
QVQLVQSGAEVKKPGASVKVSCKASGYKFTDHYMDWVRQAPGQSLEWMGYIHPHTGRTSYNQKFQGRVTMTVDRSTNTAYMELSSLRSEDTAVYYCARDSNYVNAVDYWGQGTLVTVSS

SEQ ID NO: 74 - protein, 5 aa, synthetic construct
  source 1..5: note = 70392_292 [TL1a]_H1L1 vhCDR1
DHYMD

SEQ ID NO: 75 - protein, 17 aa, synthetic construct
  source 1..17: note = 70392_292 [TL1a]_H1L1 vhCDR2
YIHPHTGRTSYNQKFQG

SEQ ID NO: 76 - protein, 10 aa, synthetic construct
  source 1..10: note = 70392_292 [TL1a]_H1L1 vhCDR3
DSNYVNAVDY

SEQ ID NO: 77 - protein, 108 aa, synthetic construct
  source 1..108: note = 70392_292 [TL1a]_H1L1 Variable Light (vl) Domain
QVVLTQSPGTLSLSPGERATLSCRASSSVSSSYLHWYQQKPGQSPRLLIYSTSNLASGVPSRFSGSGSGTDYTLTISRLEPEDFAVYYCQQYDSSPSTFGSGTKLEIK

SEQ ID NO: 78 - protein, 12 aa, synthetic construct
  source 1..12: note = 70392_292 [TL1a]_H1L1 vlCDR1
RASSSVSSSYLH

SEQ ID NO: 79 - protein, 7 aa, synthetic construct
  source 1..7: note = 70392_292 [TL1a]_H1L1 vlCDR2
STSNLAS

SEQ ID NO: 80 - protein, 9 aa, synthetic construct
  source 1..9: note = 70392_292 [TL1a]_H1L1 vlCDR3
QQYDSSPST

SEQ ID NO: 81 - protein, 251 aa, Homo sapiens
  source 1..251: note = sp|O95150|TNF15_HUMAN Tumor necrosis factor ligand superfamily member 15 OS=Homo sapiens OX=9606 GN=TNFSF15; PE = 1 SV=2
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 82 - protein, 252 aa, Mus musculus
  source 1..252: note = sp|Q5UBV8|TNF15_MOUSE Tumor necrosis factor ligand superfamily member 15 OS=Mus musculus OX=10090 GN=Tnfsf15; PE = 2 SV=2
MAEELGLGFGEGVPVEVLPEGCRHRPEARAGLAARSKACLALTCCLLSFPILAGLSTLLMAGQLRVPGKDCMLRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITVVITKVADSYPEPARLLTGSKSVCEISNNWFQSLYLGAMFSLEEGDRLMVNVSDISLVDYTKEDKTFFGAFLL

SEQ ID NO: 83 - protein, 252 aa, Rattus norvegicus
  source 1..252: note = sp|Q8K3Y7|TNF15_RAT Tumor necrosis factor ligand superfamily member 15 OS=Rattus norvegicus OX=10116; GN = Tnfsf15 PE=2 SV=1
MAEELGLGFGEAVPVEMLPEGCRHRREARTGLAARSKACLALTCCLLSFPILAGLSTLLMTGQLRIPGKDCMFPTVTEERSAPSAQPVYTPSRDKPKAHLTIMRQTPVPHLKNELAALHWENNLGMAFTKNRMNYTNKFLVIPESGDYFIYSQITFRGTTSECGDISRVRRPKKPDSITVVITKVADSYPEPAHLLTGTKSVCEISSNWFQPIYLGAMFSLEEGDRLMVNVSDISLVDYTKEDKTFFGAFLI

SEQ ID NO: 84 - protein, 216 aa, synthetic construct
  source 1..216: note = Fc backbone 1
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 216 aa, synthetic construct
  source 1..216: note = Fc backbone 2
APPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 86 - protein, 217 aa, synthetic construct
  source 1..217: note = Fc backbone 3
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 87 - protein, 98 aa, synthetic construct
  source 1..98: note = IgG1 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 88 - protein, 98 aa, synthetic construct
  source 1..98: note = IgG2 CH1
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 89 - protein, 98 aa, synthetic construct
  source 1..98: note = IgG4 CH1
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 90 - protein, 15 aa, synthetic construct
  source 1..15: note = IgG1 hinge
EPKSCDKTHTCPPCP

SEQ ID NO: 91 - protein, 12 aa, synthetic construct
  source 1..12: note = IgG2 hinge
ERKCCVECPPCP

SEQ ID NO: 92 - protein, 12 aa, synthetic construct
  source 1..12: note = IgG4 hinge
ESKYGPPCPSCP

SEQ ID NO: 93 - protein, 107 aa, synthetic construct [sequence omitted]

SEQ ID NO: 94 - protein, 106 aa, synthetic construct
  source 1..106: note = Light Chain Constant Domain - Lambda
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 95 - protein, 453 aa, synthetic construct
  source 1..453: note = XENP47498 Chain 1- 70416_041[TL1a]_H0_Fab-IgG1_PVA_/S267K
QVQLMESGPGLVQPSETLALTCTVSGFSVTSYNVHWVRQPPGKGLEWMGVMWSGGNTDYNSALKSRLTISRDTSKNQVFLKMNSLQSEDTTTYYCARAGRYYHDSNYYWDFPYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 214 aa, synthetic construct
  source 1..214: note = XENP47498 Chain 2 70416_041[TL1a]_L0_Light Chain
DIQMTQSPASLSASLGESVTIQCRASEDIYSGLAWYQQKPGKSPQLLIYGASSLQDGVPSRFSGSGSGTQYSLKISSMQTEDEGVYFCQEGLKSPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 443 aa, synthetic construct
  source 1..443: note = XENP47824 Chain 1- 70392_069[TL1a]_H1_Fab-IgG1_PVA_/S267K
EVQLVESGGGLVQPGGSLKLSCAASGFDIIDDYIHWVRQASGKGLEWVGRIDPASANTKYVPSVKGRFTISADTSKNTAYLQMNSLKTEDTAVYYCTRKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 214 aa, synthetic construct
  source 1..214: note = XENP47824 Chain 2- 70392_069[TL1a]_L1_Light Chain
NIVMTQTPLSLSVTPGQPASISCKASENVGTYVSWYLQKPGQSPQLLIYGTSNRYTGVPDRFTGSGSATDFTLKISRVEAEDVGVYHCGQSYRYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 455 aa, synthetic construct
  source 1..455: note = XENP47773 Chain 1- 70395_069[TL1a]_H1_Fab-IgG1_PVA_/S267K
QVQLVQSGAEVKKPGASVKVSCKVSGFSLARYSVTWVRQAPGQGPEWMGRMWYDGDTAYNSKFQGRVTMTRDTSTNTVYMELSSLRSEDTAVYYCTRDTRYYAGSYSQHYWYFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 214 aa, synthetic construct
  source 1..214: note = XENP47773 Chain 2- 70395_069[TL1a]_L1_Light Chain
DIQMTQSPSSLSASVGDRVTITCRASEDIYSGLAWYQQKPGKSPKLLIYGASGLQDGVPSRFSGSGSGTDYTLTISSLQPEDFATYFCQQGLKYPYTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 447 aa, synthetic construct
  source 1..447: note = XENP47781 Chain 1- 70416_092[TL1a]_H1_Fab-IgG1_PVA_/S267K
QVQLVQSGAEVKKPGASVKVSCKVSGFSLTNYGVSWVRQAPGQGLEWMGGIWGDGSTDYDSKFQGRVTMTRDTSTSTVYMELSSLRSEDTAVYFCTRYSSYTNWFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 214 aa, synthetic construct
  source 1..214: note = XENP47781 Chain 2- 70416_092[TL1a]_L1_Light Chain
DIQMTQSPSSLSASVGDRVTITCRAREDIYNGLAWYQQKPGKSPKLLIYNANRLHTGVPSRFSGSGSGTDFTLTISSLQPEDFATYFCQQYYDYPLTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 453 aa, synthetic construct
  source 1..453: note = XENP47776 Chain 1- 70416_041[TL1a]_H1_Fab-IgG1_PVA_/S267K
QVQLVQSGAEVKKPGASVKVSCKVSGFSVTSYNVHWVRQAPGQGLEWMGVMWSGGNTDYNSKFQGRVTMTRDTSTNTVYMELSSLRSEDTAVYYCARAGRYYHDSNYYWDFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 214 aa, synthetic construct
  source 1..214: note = XENP47776 Chain 2- 70416_041[TL1a]_L1_Light Chain
DIQMTQSPSSVSASVGDRVTITCRASEDIYSGLAWYQQKPGKSPKLLIYGASSLQDGVPSRFSGSGSGTDYTLTISSLQPEDFATYFCQEGLKSPYTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 446 aa, synthetic construct
  source 1..446: note = XENP47753 Chain 1- 70392_201[TL1a]_H1_Fab-IgG1_PVA_/S267K
QVQLVQSGAEVKKPGASVKVSCKASGYTFTDYYMDWVRQAPGQSLEWMGYIYPHTGRTDYNQKFQGRVTMTVDKSISTAYMELSRLRSDDTAVYFCARDSNYYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 214 aa, synthetic construct
  source 1..214: note = XENP47753 Chain 2- 70392_201[TL1a]_L1_Light Chain
DIVMTQSPLSLPVTPGEPASISCRASQNVRTAVAWYLQKPGQSPQALIYLASNRHTGVPDRFTGSGSGTDFTLKISRVEAEDVGVYFCLQHWNYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 448 aa, synthetic construct
  source 1..448: note = XENP47780 Chain 1- 70416_073[TL1a]_H1_Fab-IgG1_PVA_/S267K
QVQLVQSGAEVKKPGASVKVSCKVSGFSLNTYGVIWVRQAPGQGLEWMGVIWANGNTNYNSKLQGRVTMTRDTSTSTVYMELRSLRSDDTAVYFCARARYYYSGVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 219 aa, synthetic construct
  source 1..219: note = XENP47780 Chain 2- 70416_073[TL1a]_L1_Light Chain
DVVLTQSPLSLPVTLGQPASISCRSSQSLVHSDGNTYLDWYQQRPGQSPRLLIYKVSNRFSGVPDRFIGSGSGSDFTLKISRVEAEDVGVYYCFQATHDPWAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 448 aa, synthetic construct
  source 1..448: note = XENP47772 Chain 1- 70417_111[TL1a]_H1_Fab-IgG1_PVA_/S267K
EVQLVESGGGLVQPGGSLRLSCAASGFTFSSFGMHWVRQAPGKGLEWVSYISSGSTTIYYADSVKGRFTISRDNAKNSLYLQMNSLRAEDTAVYYCARAPLLRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 214 aa, synthetic construct
  source 1..214: note = XENP47772 Chain 2- 70417_111[TL1a]_L1_Light Chain
DIVMTQSPDSLAVSLGERATINCKASQDVSTAVVWYQQKPGQSPKLLIYWASTRHTGVPDRFTGSGSGTDYTLTISSLQAEDVAVYHCQQHYSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 443 aa, synthetic construct
  source 1..443: note = XENP47749 Chain 1- 70392_074[TL1a]_H1_Fab-IgG1_PVA_/S267K
QVQLVQSGAEVKKPGASVKVSCKASDFNIEDDYIHWVRQAPGQGLEWMGRIEPAGGNTKYAPKFQGRVTMTADTSINTAYMELSRLRSDDTAVYYCARKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 214 aa, synthetic construct
  source 1..214: note = XENP47749 Chain 2- 70392_074[TL1a]_L1_Light Chain
KIVMTQSPDSLAVSLGERATINCKASENVGTYVSWYQQKPGQSPKLLIYGTSNRYTGVPDRFTGSGSATDFTLTISSLQAEDVAVYHCGQSYSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 453 aa, synthetic construct
  source 1..453: note = XENP47782 Chain 1- 70416_094[TL1a]_H1_Fab-IgG1_PVA_/S267K
EVQLVESGGGLVKPGGSLRLSCAVSGFSLTSHSVSWVRQAPGKGPEWVGRMWYDGDTVYNSPVKGRFTISRDTSKNTLYLQMNSLKTEDTAVYYCTRNSRYYDVSYHRFVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 214 aa, synthetic construct
  source 1..214: note = XENP47782 Chain 2- 70416_094[TL1a]_L1_Light Chain
DTVLTQSPSSLSASVGDRVTITCRASESVSTGMHWYQQKPGKQPKLLIYGASNLESGVPARFSGSGSGTDFTLTISSLQPEDFATYFCQQSWSDPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 448 aa, synthetic construct
  source 1..448: note = XENP47754 Chain 1- 70392_292[TL1a]_H1_Fab-IgG1_PVA_/S267K
QVQLVQSGAEVKKPGASVKVSCKASGYKFTDHYMDWVRQAPGQSLEWMGYIHPHTGRTSYNQKFQGRVTMTVDRSTNTAYMELSSLRSEDTAVYYCARDSNYVNAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 215 aa, synthetic construct
  source 1..215: note = XENP47754 Chain 2- 70392_292[TL1a]_L1_Light Chain
QVVLTQSPGTLSLSPGERATLSCRASSSVSSSYLHWYQQKPGQSPRLLIYSTSNLASGVPSRFSGSGSGTDYTLTISRLEPEDFAVYYCQQYDSSPSTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 443 aa, synthetic construct
  source 1..443: note = XENP47952 Chain 1- 70392_069[TL1a]_H1_Fab-IgG1_PVA_/S267K/M428L/N434S
EVQLVESGGGLVQPGGSLKLSCAASGFDIIDDYIHWVRQASGKGLEWVGRIDPASANTKYVPSVKGRFTISADTSKNTAYLQMNSLKTEDTAVYYCTRKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 118 - protein, 214 aa, synthetic construct; identical to SEQ ID NO: 98
  source 1..214: note = XENP47952 Chain 2- 70392_069[TL1a]_L1_Light Chain

SEQ ID NO: 119 - protein, 455 aa, synthetic construct
  source 1..455: note = XENP47939 Chain 1- 70395_069[TL1a]_H1_Fab-IgG1_PVA_/S267K/M428L/N434S
QVQLVQSGAEVKKPGASVKVSCKVSGFSLARYSVTWVRQAPGQGPEWMGRMWYDGDTAYNSKFQGRVTMTRDTSTNTVYMELSSLRSEDTAVYYCTRDTRYYAGSYSQHYWYFDFWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 120 - protein, 214 aa, synthetic construct; identical to SEQ ID NO: 100
  source 1..214: note = XENP47939 Chain 2- 70395_069[TL1a]_L1_Light Chain

SEQ ID NO: 121 - protein, 447 aa, synthetic construct
  source 1..447: note = XENP47947 Chain 1- 70416_092[TL1a]_H1_Fab-IgG1_PVA_/S267K/M428L/N434S
QVQLVQSGAEVKKPGASVKVSCKVSGFSLTNYGVSWVRQAPGQGLEWMGGIWGDGSTDYDSKFQGRVTMTRDTSTSTVYMELSSLRSEDTAVYFCTRYSSYTNWFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 122 - protein, 214 aa, synthetic construct; identical to SEQ ID NO: 102
  source 1..214: note = XENP47947 Chain 2- 70416_092[TL1a]_L1_Light Chain

SEQ ID NO: 123 - protein, 453 aa, synthetic construct
  source 1..453: note = XENP47942 Chain 1- 70416_041[TL1a]_H1_Fab-IgG1_PVA_/S267K/M428L/N434S
QVQLVQSGAEVKKPGASVKVSCKVSGFSVTSYNVHWVRQAPGQGLEWMGVMWSGGNTDYNSKFQGRVTMTRDTSTNTVYMELSSLRSEDTAVYYCARAGRYYHDSNYYWDFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 124 - protein, 214 aa, synthetic construct; identical to SEQ ID NO: 104
  source 1..214: note = XENP47942 Chain 2- 70416_041[TL1a]_L1_Light Chain

SEQ ID NO: 125 - protein, 446 aa, synthetic construct
  source 1..446: note = XENP47919 Chain 1- 70392_201[TL1a]_H1_Fab-IgG1_PVA_/S267K/M428L/N434S
QVQLVQSGAEVKKPGASVKVSCKASGYTFTDYYMDWVRQAPGQSLEWMGYIYPHTGRTDYNQKFQGRVTMTVDKSISTAYMELSRLRSDDTAVYFCARDSNYYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 126 - protein, 214 aa, synthetic construct; identical to SEQ ID NO: 106
  source 1..214: note = XENP47919 Chain 2- 70392_201[TL1a]_L1_Light Chain

SEQ ID NO: 127 - protein, 448 aa, synthetic construct
  source 1..448: note = XENP47946 Chain 1- 70416_073[TL1a]_H1_Fab-IgG1_PVA_/S267K/M428L/N434S
QVQLVQSGAEVKKPGASVKVSCKVSGFSLNTYGVIWVRQAPGQGLEWMGVIWANGNTNYNSKLQGRVTMTRDTSTSTVYMELRSLRSDDTAVYFCARARYYYSGVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 128 - protein, 219 aa, synthetic construct; identical to SEQ ID NO: 108
  source 1..219: note = XENP47946 Chain 2- 70416_073[TL1a]_L1_Light Chain

SEQ ID NO: 129 - protein, 448 aa, synthetic construct
  source 1..448: note = XENP47938 Chain 1- 70417_111[TL1a]_H1_Fab-IgG1_PVA_/S267K/M428L/N434S
EVQLVESGGGLVQPGGSLRLSCAASGFTFSSFGMHWVRQAPGKGLEWVSYISSGSTTIYYADSVKGRFTISRDNAKNSLYLQMNSLRAEDTAVYYCARAPLLRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 130 - protein, 214 aa, synthetic construct; identical to SEQ ID NO: 110
  source 1..214: note = XENP47938 Chain 2- 70417_111[TL1a]_L1_Light Chain

SEQ ID NO: 131 - protein, 443 aa, synthetic construct
  source 1..443: note = XENP47915 Chain 1- 70392_074[TL1a]_H1_Fab-IgG1_PVA_/S267K/M428L/N434S
QVQLVQSGAEVKKPGASVKVSCKASDFNIEDDYIHWVRQAPGQGLEWMGRIEPAGGNTKYAPKFQGRVTMTADTSINTAYMELSRLRSDDTAVYYCARKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 132 - protein, 214 aa, synthetic construct; identical to SEQ ID NO: 112
  source 1..214: note = XENP47915 Chain 2- 70392_074[TL1a]_L1_Light Chain

SEQ ID NO: 133 - protein, 453 aa, synthetic construct
  source 1..453: note = XENP47948 Chain 1- 70416_094[TL1a]_H1_Fab-IgG1_PVA_/S267K/M428L/N434S
EVQLVESGGGLVKPGGSLRLSCAVSGFSLTSHSVSWVRQAPGKGPEWVGRMWYDGDTVYNSPVKGRFTISRDTSKNTLYLQMNSLKTEDTAVYYCTRNSRYYDVSYHRFVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 134 - protein, 214 aa, synthetic construct; identical to SEQ ID NO: 114
  source 1..214: note = XENP47948 Chain 2- 70416_094[TL1a]_L1_Light Chain

SEQ ID NO: 135 - protein, 448 aa, synthetic construct
  source 1..448: note = XENP47920 Chain 1- 70392_292[TL1a]_H1_Fab-IgG1_PVA_/S267K/M428L/N434S
QVQLVQSGAEVKKPGASVKVSCKASGYKFTDHYMDWVRQAPGQSLEWMGYIHPHTGRTSYNQKFQGRVTMTVDRSTNTAYMELSSLRSEDTAVYYCARDSNYVNAVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 136 - protein, 215 aa, synthetic construct; identical to SEQ ID NO: 116
  source 1..215: note = XENP47920 Chain 2- 70392_292[TL1a]_L1_Light Chain

SEQ ID NO: 137 - protein, 454 aa, synthetic construct
  source 1..454: note = XENP48083 Chain 1- 70416_041[TL1a]_H1_Fab-IgG1_L234A/L235A/P329A/M428L/N434S
QVQLVQSGAEVKKPGASVKVSCKVSGFSVTSYNVHWVRQAPGQGLEWMGVMWSGGNTDYNSKFQGRVTMTRDTSTNTVYMELSSLRSEDTAVYYCARAGRYYHDSNYYWDFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 138 - protein, 214 aa, synthetic construct; identical to SEQ ID NO: 104
  source 1..214: note = XENP48083 Chain 2 70416_041[TL1a]_L1_Light Chain

SEQ ID NO: 139 - protein, 449 aa, synthetic construct
  source 1..449: note = XENP48058 Chain 1- 70392_189[TL1a]_H1_Fab-IgG1_L234A/L235A/P329A/M428L/N434S
QVQLVQSGAEVKKPGASVKVSCKASGYTFTDYEIHWMRQAPGQGLEWMGGIDPETGGTAYNQKFQGRVTMTADKSISTAYMELSRLRSDDTAVYYCTRFLVHHYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 140 - protein, 219 aa, synthetic construct
  source 1..219: note = XENP48058 Chain 2- 70392_189[TL1a]_L1_Light Chain
DVVMTQSPLSLPVTLGQPASISCRSSQSLLDSDGKTYLNWLQKRPGQSPRRLIYLVSKLDSGVPDRFTGSGSGTDFTLKISRVEAEDVGVYYCWQGTQLPFTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 444 aa, synthetic construct
  source 1..444: note = XENP48093 Chain 1- 70392_069[TL1a]_H1_Fab-IgG1_L234A/L235A/P329A/M428L/N434S
EVQLVESGGGLVQPGGSLKLSCAASGFDIIDDYIHWVRQASGKGLEWVGRIDPASANTKYVPSVKGRFTISADTSKNTAYLQMNSLKTEDTAVYYCTRKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 142 - protein, 214 aa, synthetic construct; identical to SEQ ID NO: 98
  source 1..214: note = XENP48093 Chain 2- 70392_069[TL1a]_L1_Light Chain

SEQ ID NO: 143 - protein, 448 aa, synthetic construct
  source 1..448: note = XENP48088 Chain 1- 70416_092[TL1a]_H1_Fab-IgG1_L234A/L235A/P329A/M428L/N434S
QVQLVQSGAEVKKPGASVKVSCKVSGFSLTNYGVSWVRQAPGQGLEWMGGIWGDGSTDYDSKFQGRVTMTRDTSTSTVYMELSSLRSEDTAVYFCTRYSSYTNWFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 144 - protein, 214 aa, synthetic construct; identical to SEQ ID NO: 102
  source 1..214: note = XENP48088 Chain 2- 70416_092[TL1a]_L1_Light Chain

SEQ ID NO: 145 - protein, 449 aa, synthetic construct
  source 1..449: note = XENP48087 Chain 1 - 70416_073[TL1a]_H1_Fab-IgG1_L234A/L235A/P329A/M428L/N434S
QVQLVQSGAEVKKPGASVKVSCKVSGFSLNTYGVIWVRQAPGQGLEWMGVIWANGNTNYNSKLQGRVTMTRDTSTSTVYMELRSLRSDDTAVYFCARARYYYSGVLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 146 - protein, 219 aa, synthetic construct; identical to SEQ ID NO: 108
  source 1..219: note = XENP48087 Chain 2- 70416_073[TL1a]_L1_Light Chain

SEQ ID NO: 147 - protein, 448 aa, synthetic construct; identical to SEQ ID NO: 129
  source 1..448: note = XENP47938 Chain 1- 70417_111[TL1a]_H1_Fab-IgG1_PVA_/S267K/M428L/N434S

SEQ ID NO: 148 - protein, 214 aa, synthetic construct; identical to SEQ ID NO: 110
  source 1..214: note = XENP47938 Chain 2- 70417_111[TL1a]_L1_Light Chain

SEQ ID NO: 149 - protein, 444 aa, synthetic construct
  source 1..444: note = XENP48056 Chain 1- 70392_074[TL1a]_H1_Fab-IgG1_L234A/L235A/P329A/M428L/N434S
QVQLVQSGAEVKKPGASVKVSCKASDFNIEDDYIHWVRQAPGQGLEWMGRIEPAGGNTKYAPKFQGRVTMTADTSINTAYMELSRLRSDDTAVYYCARKGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 150 - protein, 214 aa, synthetic construct; identical to SEQ ID NO: 112
  source 1..214: note = XENP48056 Chain 2- 70392_074[TL1a]_L1_Light Chain

SEQ ID NO: 151 - protein, 447 aa, synthetic construct
  source 1..447: note = XENP48060 Chain 1- 70392_201[TL1a]_H1_Fab-IgG1_L234A/L235A/P329A/M428L/N434S
QVQLVQSGAEVKKPGASVKVSCKASGYTFTDYYMDWVRQAPGQSLEWMGYIYPHTGRTDYNQKFQGRVTMTVDKSISTAYMELSRLRSDDTAVYFCARDSNYYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 152 - protein, 214 aa, synthetic construct; identical to SEQ ID NO: 106
  source 1..214: note = XENP48060 Chain 2- 70392_201[TL1a]_L1_Light Chain

SEQ ID NO: 153 - protein, 454 aa, synthetic construct
  source 1..454: note = XENP48089 Chain 1-70416_094[TL1a]_H1_Fab-IgG1_L234A /L235A/P329A/M428L/N434S
EVQLVESGGGLVKPGGSLRLSCAVSGFSLTSHSVSWVRQAPGKGPEWVGRMWYDGDTVYNSPVKGRFTISRDTSKNTLYLQMNSLKTEDTAVYYCTRNSRYYDVSYHRFVMDAWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 154 - protein, 214 aa, synthetic construct; identical to SEQ ID NO: 114
  source 1..214: note = XENP48089 Chain 2- 70416_094[TL1a]_L1_Light Chain

SEQ ID NO: 155 - protein, 116 aa, synthetic construct
  source 1..116: note = Comparator 1 [TL1A]_H0L0 Variable Heavy (vh) Domain
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 156 - protein, 5 aa, synthetic construct
  source 1..5: note = Comparator 1 [TL1A]_H0L0 vhCDR1
DTYMH

SEQ ID NO: 157 - protein, 17 aa, synthetic construct
  source 1..17: note = Comparator 1 [TL1A]_H0L0 vhCDR2
RIDPASGHTKYDPKFQV

SEQ ID NO: 158 - protein, 7 aa, synthetic construct
  source 1..7: note = Comparator 1 [TL1A]_H0L0 vhCDR3
SGGLPDV

SEQ ID NO: 159 - protein, 106 aa, synthetic construct
  source 1..106: note = Comparator 1 [TL1A]_H0L0 Variable Light (vl) Domain
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 160 - protein, 10 aa, synthetic construct
  source 1..10: note = Comparator 1 [TL1A]_H0L0 vlCDR1
RASSSVSYMY

SEQ ID NO: 161 - protein, 7 aa, synthetic construct
  source 1..7: note = Comparator 1 [TL1A]_H0L0 vlCDR2
ATSNLAS

SEQ ID NO: 162 - protein, 9 aa, synthetic construct
  source 1..9: note = Comparator 1 [TL1A]_H0L0 vlCDR3
QQWEGNPRT

SEQ ID NO: 163 - protein, 124 aa, synthetic construct
  source 1..124: note = Comparator 2 [TL1A]_H0L0 Variable Heavy (vh) Domain
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 164 - protein, 5 aa, synthetic construct
  source 1..5: note = Comparator 2 [TL1A]_H0L0 vhCDR1
YYGIS

SEQ ID NO: 165 - protein, 17 aa, synthetic construct
  source 1..17: note = Comparator 2 [TL1A]_H0L0 vhCDR2
WISTYNGNTHYARMLQG

SEQ ID NO: 166 - protein, 15 aa, synthetic construct
  source 1..15: note = Comparator 2 [TL1A]_H0L0 vhCDR3
ENYYGSGAYRGGMDV

SEQ ID NO: 167 - protein, 107 aa, synthetic construct
  source 1..107: note = Comparator 2 [TL1A]_H0L0 Variable Light (vl) Domain
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 168 - protein, 11 aa, synthetic construct
  source 1..11: note = Comparator 2 [TL1A]_H0L0 vlCDR1
RASQSVSSYLA

SEQ ID NO: 169 - protein, 7 aa, synthetic construct
  source 1..7: note = Comparator 2 [TL1A]_H0L0 vlCDR2
DASNRAT

SEQ ID NO: 170 - protein, 9 aa, synthetic construct
  source 1..9: note = Comparator 2 [TL1A]_H0L0 vlCDR3
QQRSNWPWT

SEQ ID NO: 171 - protein, 119 aa, synthetic construct
  source 1..119: note = Comparator 3-1 [TL1A]_H0L0 Variable Heavy (vh) Domain
QVQLVQSGAEVKKPGASVKVSCKASGYTFTSYDINWVRQAPGQGLEWMGWLNPNSGNTGYAQKFQGRVTMTRNTSISTAYMELSSLRSEDTAVYYCAREVPETAAFEYWGQGTLVTVSS

SEQ ID NO: 172 - protein, 5 aa, synthetic construct
  source 1..5: note = Comparator 3-1 [TL1A]_H0L0 vhCDR1
SYDIN

SEQ ID NO: 173 - protein, 16 aa, synthetic construct
  source 1..16: note = Comparator 3-1 [TL1A]_H0L0 vhCDR2
WLNPNSGNTGYAQKFQ

SEQ ID NO: 174 - protein, 10 aa, synthetic construct
  source 1..10: note = Comparator 3-1 [TL1A]_H0L0 vhCDR3
EVPETAAFEY

SEQ ID NO: 175 - protein, 111 aa, synthetic construct
  source 1..111: note = Comparator 3-1 [TL1A]_H0L0 Variable Light (vl) Domain
QSVLTQPPSVSGAPGQRVTISCTSSSSDIGAGLGVHWYQQLPGTAPKLLIYGYYNRPSGVPDRFSGSKSGTSASLTITGLLPEDEGDYYCQSYDGTLSALFGGGTKLTVLG

SEQ ID NO: 176 - protein, 14 aa, synthetic construct
  source 1..14: note = Comparator 3-1 [TL1A]_H0L0 vlCDR1
TSSSSDIGAGLGVH

SEQ ID NO: 177 - protein, 7 aa, synthetic construct
  source 1..7: note = Comparator 3-1 [TL1A]_H0L0 vlCDR2
GYYNRPS

SEQ ID NO: 178 - protein, 10 aa, synthetic construct
  source 1..10: note = Comparator 3-1 [TL1A]_H0L0 vlCDR3
QSYDGTLSAL

SEQ ID NO: 179 - protein, 119 aa, synthetic construct
  source 1..119: note = Comparator 3-2 [TL1A]_H0L0 Variable Heavy (vh) Domain
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSS

SEQ ID NO: 180 - protein, 5 aa, synthetic construct
  source 1..5: note = Comparator 3-2 [TL1A]_H0L0 vhCDR1
SYDIN

SEQ ID NO: 181 - protein, 17 aa, synthetic construct
  source 1..17: note = Comparator 3-2 [TL1A]_H0L0 vhCDR2
WLNPNSGNTGYAQKFQG

SEQ ID NO: 182 - protein, 10 aa, synthetic construct
  source 1..10: note = Comparator 3-2 [TL1A]_H0L0 vhCDR3
EVPETAAFEY

SEQ ID NO: 183 - protein, 111 aa, synthetic construct
  source 1..111: note = Comparator 3-2 [TL1A]_H0L0 Variable Light (vl) Domain
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLG

SEQ ID NO: 184 - protein, 14 aa, synthetic construct
  source 1..14: note = Comparator 3-2 [TL1A]_H0L0 vlCDR1
TSSSSDIGAGLGVH

SEQ ID NO: 185 - protein, 7 aa, synthetic construct
  source 1..7: note = Comparator 3-2 [TL1A]_H0L0 vlCDR2
GYYNRPS

SEQ ID NO: 186 - protein, 10 aa, synthetic construct
  source 1..10: note = Comparator 3-2 [TL1A]_H0L0 vlCDR3
QSYDGTLSAL

SEQ ID NO: 187 - protein, 119 aa, synthetic construct
  source 1..119: note = Comparator 3-3 [TL1A]_H0L0 Variable Heavy (vh) Domain
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 188 - protein, 5 aa, synthetic construct
  source 1..5: note = Comparator 3-3 [TL1A]_H0L0 vhCDR1
SYDIN

SEQ ID NO: 189 - protein, 17 aa, synthetic construct
  source 1..17: note = Comparator 3-3 [TL1A]_H0L0 vhCDR2
WLNPNSGYTGYAQKFQG

SEQ ID NO: 190 - protein, 10 aa, synthetic construct
  source 1..10: note = Comparator 3-3 [TL1A]_H0L0 vhCDR3
EVPETAAFEY

SEQ ID NO: 191 - protein, 111 aa, synthetic construct
  source 1..111: note = Comparator 3-3 [TL1A]_H0L0 Variable Light (vl) Domain
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLG

SEQ ID NO: 192 - protein, 14 aa, synthetic construct
  source 1..14: note = Comparator 3-3 [TL1A]_H0L0 vlCDR1
TSSSSDIGAGLGVH

SEQ ID NO: 193 - protein, 7 aa, synthetic construct
  source 1..7: note = Comparator 3-3 [TL1A]_H0L0 vlCDR2
GYYNRPS

SEQ ID NO: 194 - protein, 10 aa, synthetic construct
  source 1..10: note = Comparator 3-3 [TL1A]_H0L0 vlCDR3
QSWDGTLSAL

SEQ ID NO: 195 - protein, 119 aa, synthetic construct
  source 1..119: note = Comparator 4 [TL1A]_H0L0 Variable Heavy (vh) Domain
EVQLVESGGGLVKPGGSLRLSCAASGFTFSNAWMTWVRQAPGKGLEWVGRIKSKIDAGTTDYAAPVKGRFTISRDDSKNTLYLQMNSLKTEDTAVYYCTTDLGWGENYWGQGTLVTVSS

SEQ ID NO: 196 - protein, 5 aa, synthetic construct
  source 1..5: note = Comparator 4 [TL1A]_H0L0 vhCDR1
NAWMT

SEQ ID NO: 197 - protein, 19 aa, synthetic construct
  source 1..19: note = Comparator 4 [TL1A]_H0L0 vhCDR2
RIKSKIDAGTTDYAAPVKG

SEQ ID NO: 198 - protein, 8 aa, synthetic construct
  source 1..8: note = Comparator 4 [TL1A]_H0L0 vhCDR3
DLGWGENY

SEQ ID NO: 199 - protein, 108 aa, synthetic construct
  source 1..108: note = Comparator 4 [TL1A]_H0L0 Variable Light (vl) Domain
ENVLTQSPGTLSLSPGERATLSCRASQRVSSSYLVWYQQKPGQAPRLLIYGASSRATGIPDRFSGSGSGTDFTLTISRLEPEDFAVYYCQQYGSSPYTFGQGTKLEIK

SEQ ID NO: 200 - protein, 12 aa, synthetic construct
  source 1..12: note = Comparator 4 [TL1A]_H0L0 vlCDR1
RASQRVSSSYLV

SEQ ID NO: 201 - protein, 7 aa, synthetic construct
  source 1..7: note = Comparator 4 [TL1A]_H0L0 vlCDR2
GASSRAT

SEQ ID NO: 202 - protein, 9 aa, synthetic construct
  source 1..9: note = Comparator 4 [TL1A]_H0L0 vlCDR3
QQYGSSPYT

SEQ ID NO: 203 - protein, 118 aa, synthetic construct
  source 1..118: note = Comparator 5 [TL1A]_H0L0 Variable Heavy (vh) Domain
EVQLVQSGAEVKKPGASVKVSCKASGYTFTGYYMHWVRQAPGQGLEWMGWINPKSGGTNYAQKFQGRVTMTRDTSISTAYMELSRLISDDTAVYYCATGGSFDAFDVWGQGTMVTVSS

SEQ ID NO: 204 - protein, 5 aa, synthetic construct
  source 1..5: note = Comparator 5 [TL1A]_H0L0 vhCDR1
GYYMH

SEQ ID NO: 205 - protein, 17 aa, synthetic construct
  source 1..17: note = Comparator 5 [TL1A]_H0L0 vhCDR2
WINPKSGGTNYAQKFQG

SEQ ID NO: 206 - protein, 9 aa, synthetic construct
  source 1..9: note = Comparator 5 [TL1A]_H0L0 vhCDR3
GGSFDAFDV

SEQ ID NO: 207 - protein, 107 aa, synthetic construct
  source 1..107: note = Comparator 5 [TL1A]_H0L0 Variable Light (vl) Domain
DIVMTQSPSSLSASVGDRVTITCRASQSISSYLNWYQQSPGKAPKILIYGASRLQSGVPSRFSGSGSGTDFTLAISSLQPEDFATYYCQQGDNTPFTFGPGTKLEIK

SEQ ID NO: 208 - protein, 11 aa, synthetic construct
  source 1..11: note = Comparator 5 [TL1A]_H0L0 vlCDR1
RASQSISSYLN

SEQ ID NO: 209 - protein, 7 aa, synthetic construct
  source 1..7: note = Comparator 5 [TL1A]_H0L0 vlCDR2
GASRLQS

SEQ ID NO: 210 - protein, 9 aa, synthetic construct
  source 1..9: note = Comparator 5 [TL1A]_H0L0 vlCDR3
QQGDNTPFT

SEQ ID NO: 211 - protein, 446 aa, synthetic construct
  source 1..446: note = XENP46602 Chain 1- Comparator 1 [TL1a]_H0_Fab-COMP1_IgG1_FcKO
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 213 aa, synthetic construct
  source 1..213: note = XENP46602 Chain 2- Comparator 1 [TL1a]_L0_Light Chain
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 213 - protein, 445 aa, synthetic construct
  source 1..445: note = XENP47202 Chain 1- Comparator 1 [TL1a]_H0_Fab-mIgG2aa_L234A/L235A/P329G
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPQHQDWMSGKEFKCKVNNKALGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 214 - protein, 213 aa, synthetic construct
  source 1..213: note = XENP47202 Chain 2- Comparator 1 [TL1a]_L0_Light Chain
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 215 - protein, 445 aa, synthetic construct
  source 1..445: note = XENP46601 Chain 1- Comparator 1 [TL1a]_H0_Fab-IgG1_PVA_/S267K/M428L/N434S
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 216 - protein, 213 aa, synthetic construct; identical to SEQ ID NO: 212
  source 1..213: note = XENP46601 Chain 2- Comparator 1 [TL1a]_L0_Light Chain

SEQ ID NO: 217 - protein, 445 aa, synthetic construct
  source 1..445: note = XENP46600 Chain 1- Comparator 1 [TL1a]_H0_Fab-IgG1_PVA_/S267K
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 213 aa, synthetic construct; identical to SEQ ID NO: 212
  source 1..213: note = XENP46600 Chain 2- Comparator 1 [TL1a]_L0_Light Chain

SEQ ID NO: 219 - protein, 453 aa, synthetic construct
  source 1..453: note = XENP46598 Chain 1- Comparator 2 [TL1a]_H0_Fab-IgG1_PVA_/S267K/M428L/N434S
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 220 - protein, 214 aa, synthetic construct
  source 1..214: note = XENP46598 Chain 2- Comparator 2 [TL1a]_L0_Light Chain
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 221 - protein, 454 aa, synthetic construct
  source 1..454: note = XENP46599 Chain 1- Comparator 2 [TL1a]_H0_Fab-COMP2_IgG1_FcKO
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 214 aa, synthetic construct; identical to SEQ ID NO: 220
  source 1..214: note = XENP46599 Chain 2- Comparator 2 [TL1a]_L0_Light Chain

SEQ ID NO: 223 - protein, 453 aa, synthetic construct
  source 1..453: note = XENP46597 Chain 1- Comparator 2 [TL1a]_H0_Fab-IgG1_PVA_/S267K
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 214 aa, synthetic construct; identical to SEQ ID NO: 220
  source 1..214: note = XENP46597 Chain 2- Comparator 2 [TL1a]_L0_Light Chain

SEQ ID NO: 225 - protein, 448 aa, synthetic construct
  source 1..448: note = XENP46741 Chain 1- Comparator 3-1 [TL1a]_H0_Fab-IgG1_PVA_/S267K
QVQLVQSGAEVKKPGASVKVSCKASGYTFTSYDINWVRQAPGQGLEWMGWLNPNSGNTGYAQKFQGRVTMTRNTSISTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 216 aa, synthetic construct
  source 1..216: note = XENP46741 Chain 2- Comparator 3-1 [TL1a]_L0_Light Chain
QSVLTQPPSVSGAPGQRVTISCTSSSSDIGAGLGVHWYQQLPGTAPKLLIYGYYNRPSGVPDRFSGSKSGTSASLTITGLLPEDEGDYYCQSYDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 227 - protein, 448 aa, synthetic construct
  source 1..448: note = XENP46742 Chain 1- Comparator 3-2[TL1a]_H0_Fab-IgG1_PVA_/S267K
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 216 aa, synthetic construct
  source 1..216: note = XENP46742 Chain 2- Comparator 3-2 [TL1a]_L0_Light Chain
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSYDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 229 - protein, 449 aa, synthetic construct
  source 1..449: note = XENP48569 Chain 1- Comparator 3-1 [TL1a]_H0_Fab-IgG1_L234A/L235A/P329A/M428L/N434S
QVQLVQSGAEVKKPGASVKVSCKASGYTFTSYDINWVRQAPGQGLEWMGWLNPNSGNTGYAQKFQGRVTMTRNTSISTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 230 - protein, 216 aa, synthetic construct; identical to SEQ ID NO: 226
  source 1..216: note = XENP48569 Chain 2- Comparator 3-1 [TL1a]_L0_Light Chain

SEQ ID NO: 231 - protein, 449 aa, synthetic construct
  source 1..449: note = XENP48570 Chain 1- Comparator 3-2 [TL1a]_H0_Fab-IgG1_L234A/L235A/P329A/M428L/N434S
QVQLVQSGAEVKKPGASVKVSCKASGYTFTSYDINWVRQAPGQGLEWMGWLNPNSGNTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 232 - protein, 216 aa, synthetic construct; identical to SEQ ID NO: 228
  source 1..216: note = XENP48570 Chain 2- Comparator 3-2 [TL1a]_L0_Light Chain

SEQ ID NO: 233 - protein, 448 aa, synthetic construct
  source 1..448: note = XENP49724 Chain 1- Comparator 3-3[TL1a]H1_Fab-IgG1_replica_E356D/M358L/delK447
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 234 - protein, 216 aa, synthetic construct
  source 1..216: note = XENP49724 Chain 2- Comparator 3-3 [TL1a]L1_Light Chain
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 235 - protein, 449 aa, synthetic construct
  source 1..449: note = XENPXXXX Chain 1- Comparator 4 [TL1a]_H0_Fab-COMP1_IgG1_FcKO
EVQLVESGGGLVKPGGSLRLSCAASGFTFSNAWMTWVRQAPGKGLEWVGRIKSKIDAGTTDYAAPVKGRFTISRDDSKNTLYLQMNSLKTEDTAVYYCTTDLGWGENYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 215 aa, synthetic construct
  source 1..215: note = XENPXXXX Chain 2- Comparator 4 [TL1a]_L0_Light Chain
ENVLTQSPGTLSLSPGERATLSCRASQRVSSSYLVWYQQKPGQAPRLLIYGASSRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 237 - protein, 448 aa, synthetic construct
  source 1..448: note = XENPXXXXX Chain 1- Comparator 4 [TL1a]_H0_Fab-mIgG2aa_L234A/L235A/P329G
EVQLVESGGGLVKPGGSLRLSCAASGFTFSNAWMTWVRQAPGKGLEWVGRIKSKIDAGTTDYAAPVKGRFTISRDDSKNTLYLQMNSLKTEDTAVYYCTTDLGWGENYWGQGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPQHQDWMSGKEFKCKVNNKALGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 238 - protein, 215 aa, synthetic construct
  source 1..215: note = XENPXXXXX Chain 2- Comparator 4 [TL1a]_L0_Light Chain
ENVLTQSPGTLSLSPGERATLSCRASQRVSSSYLVWYQQKPGQAPRLLIYGASSRATGIPDRFSGSGSGTDFTLTISRLEPEDFAVYYCQQYGSSPYTFGQGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 239 - protein, 448 aa, synthetic construct
  source 1..448: note = XENPXXXXX Chain 1- Comparator 4 [TL1a]_H0_Fab-IgG1_PVA_/S267K/M428L/N434S
EVQLVESGGGLVKPGGSLRLSCAASGFTFSNAWMTWVRQAPGKGLEWVGRIKSKIDAGTTDYAAPVKGRFTISRDDSKNTLYLQMNSLKTEDTAVYYCTTDLGWGENYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 240 - protein, 215 aa, synthetic construct; identical to SEQ ID NO: 236
  source 1..215: note = XENPXXXXX Chain 2- Comparator 4 [TL1a]_L0_Light Chain

SEQ ID NO: 241 - protein, 448 aa, synthetic construct
  source 1..448: note = XENPXXXXX Chain 1- Comparator 4 [TL1a]_H0_Fab-IgG1_PVA_/S267K
EVQLVESGGGLVKPGGSLRLSCAASGFTFSNAWMTWVRQAPGKGLEWVGRIKSKIDAGTTDYAAPVKGRFTISRDDSKNTLYLQMNSLKTEDTAVYYCTTDLGWGENYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 215 aa, synthetic construct; identical to SEQ ID NO: 236
  source 1..215: note = XENPXXXXX Chain 2- Comparator 4 [TL1a]_L0_Light Chain

SEQ ID NO: 243 - protein, 448 aa, synthetic construct
  source 1..448: note = XENPXXXX Chain 1- Comparator 5 [TL1a]_H0_Fab-COMP1_IgG1_FcKO
EVQLVQSGAEVKKPGASVKVSCKASGYTFTGYYMHWVRQAPGQGLEWMGWINPKSGGTNYAQKFQGRVTMTRDTSISTAYMELSRLISDDTAVYYCATGGSFDAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 214 aa, synthetic construct
  source 1..214: note = XENPXXXX Chain 2- Comparator 5 [TL1a]_L0_Light Chain
DIVMTQSPSSLSASVGDRVTITCRASQSISSYLNWYQQSPGKAPKILIYGASRLQSGVPSRFSGSGSGTDFTLAISSLQPEDFATYYCQQGDNTPFTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 447 aa, synthetic construct
  source 1..447: note = XENPXXXXX Chain 1- Comparator 5 [TL1a]_H0_Fab-mIgG2aa_L234A/L235A/P329G
EVQLVQSGAEVKKPGASVKVSCKASGYTFTGYYMHWVRQAPGQGLEWMGWINPKSGGTNYAQKFQGRVTMTRDTSISTAYMELSRLISDDTAVYYCATGGSFDAFDVWGQGTMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNAAGGPSVFIFPPKIKDVLMISLSPMVTCVVVDVSEDDPDVQISWFVNNVEVLTAQTQTHREDYNSTLRVVSALPQHQDWMSGKEFKCKVNNKALGAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 246 - protein, 214 aa, synthetic construct
  source 1..214: note = XENPXXXXX Chain 2- Comparator 5 [TL1a]_L0_Light Chain
DIVMTQSPSSLSASVGDRVTITCRASQSISSYLNWYQQSPGKAPKILIYGASRLQSGVPSRFSGSGSGTDFTLAISSLQPEDFATYYCQQGDNTPFTFGPGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 247 - protein, 447 aa, synthetic construct
  source 1..447: note = XENPXXXXX Chain 1- Comparator 5 [TL1a]_H0_Fab-IgG1_PVA_/S267K/M428L/N434S
EVQLVQSGAEVKKPGASVKVSCKASGYTFTGYYMHWVRQAPGQGLEWMGWINPKSGGTNYAQKFQGRVTMTRDTSISTAYMELSRLISDDTAVYYCATGGSFDAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 248 - protein, 214 aa, synthetic construct; identical to SEQ ID NO: 244
  source 1..214: note = XENPXXXXX Chain 2- Comparator 5 [TL1a]_L0_Light Chain

SEQ ID NO: 249 - protein, 447 aa, synthetic construct
  source 1..447: note = XENPXXXXX Chain 1- Comparator 5 [TL1a]_H0_Fab-IgG1_PVA_/S267K
EVQLVQSGAEVKKPGASVKVSCKASGYTFTGYYMHWVRQAPGQGLEWMGWINPKSGGTNYAQKFQGRVTMTRDTSISTAYMELSRLISDDTAVYYCATGGSFDAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVKHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 214 aa, synthetic construct; identical to SEQ ID NO: 244
  source 1..214: note = XENPXXXXX Chain 2- Comparator 5 [TL1a]_L0_Light Chain

(19) United States
(12) Patent Application Publication
(10) Pub. No.: US 2025/0361311 A1
(43) Pub. Date: Nov. 27, 2025

(54) ANTI-TNF-LIKE LIGAND 1A (TL1A) ANTIBODIES

(71) Applicant: Xencor, Inc., Pasadena, CA (US)

(72) Inventors: Kendra N. Avery, Hawthorne, CA (US); Matthew S. Faber, Glendora, CA (US); James Ernst, Pasadena, CA (US)

(21) Appl. No.: 19/218,143

(22) Filed: May 23, 2025

Related U.S. Application Data
(60) Provisional application No. 63/651,887, filed on May 24, 2024.
(60) Provisional application No. 63/692,671, filed on Sep. 9, 2024.
(60) Provisional application No. 63/705,956, filed on Oct. 10, 2024.
(60) Provisional application No. 63/760,574, filed on Feb. 19, 2025.
(60) Provisional application No. 63/761,833, filed on Feb. 21, 2025.
(60) Provisional application No. 63/796,265, filed on Apr. 28, 2025.
(60) Provisional application No. 63/796,998, filed on Apr. 29, 2025.

(51) Int. Cl. C07K 16/28; A61K 39/00

(52) U.S. Cl. CPC C07K 16/2875; A61K 2039/505; C07K 2317/35; C07K 2317/55; C07K 2317/76; C07K 2317/92; C07K 2317/94

(57) ABSTRACT

Provided herein are novel TNF-Like Ligand 1A (TL1A) binding domains and antibodies that include such binding domains. The TL1A binding domains and antibodies provided herein are useful, for example, in the treatment of TL1A-associated diseases.

### PRIORITY CLAIM

This application claims priority to U.S. Provisional Patent Application Nos. 63/651,887, filed May 24, 2024; 63/692,671, filed Sep. 9, 2024; 63/705,956, filed Oct. 10, 2024; 63/760,574, filed Feb. 19, 2025; 63/796,265, filed Apr. 28, 2025; 63/761,833, filed Feb. 21, 2025; and 63/796,998, filed Apr. 29, 2025, which are hereby incorporated by reference in their entirety for all intents and purposes.

### SEQUENCE LISTING

The instant application contains a Sequence Listing which has been submitted electronically in XML format and is hereby incorporated by reference in its entirety. Said XML copy, created on May 23, 2025, is named 067461-5325-WO_SL.xml and is 307,303 bytes in size.

### BACKGROUND

Antibody-based therapeutics have been used successfully to treat a variety of diseases. There remains a need for novel therapeutics for the treatment of diseases caused by aberrant TNF-like ligand 1A (TL1A) signaling activity, particularly those that target TNF-like ligand 1A (TL1A).

### SUMMARY

Provided herein are novel TNF-Like Ligand 1A (TL1A) binding domains and antibodies that include such binding domains. The TL1A binding domains and antibodies provided herein are useful, for example, in the treatment of TL1A-associated diseases.

In one aspect, provided herein is an antibody comprising a TNF-like ligand 1A (TL1A) antigen binding domain, the TL1A binding domain comprising a variable light domain and a variable heavy domain selected from the following sets of variable light domains and variable heavy domains:
a) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
b) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
c) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
d) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
e) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
f) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
g) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
h) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
i) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
j) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80.

In another aspect, provided herein is an antibody comprising a TNF-like ligand 1A (TL1A) antigen binding domain, the TL1A binding domain comprising a variable heavy domain and a variable light domain selected from the following sets of variable heavy domains and variable light domains:
a) a variable heavy domain comprising a vhCDR1, vhCDR2, and vhCDR3 of a variable heavy domain having an amino acid sequence of SEQ ID NO:1, and a variable light domain comprising a vlCDR1, vlCDR2, and vlCDR3 of a variable light domain having an amino acid sequence of SEQ ID NO:5;
b) a variable heavy domain comprising a vhCDR1, vhCDR2, and vhCDR3 of a variable heavy domain having an amino acid sequence of SEQ ID NO:9, and a variable light domain comprising a vlCDR1, vlCDR2, and vlCDR3 of a variable light domain having an amino acid sequence of SEQ ID NO:13;
c) a variable heavy domain comprising a vhCDR1, vhCDR2, and vhCDR3 of a variable heavy domain having an amino acid sequence of SEQ ID NO:17, and a variable light domain comprising a vlCDR1, vlCDR2, and vlCDR3 of a variable light domain having an amino acid sequence of SEQ ID NO:21;
d) a variable heavy domain comprising a vhCDR1, vhCDR2, and vhCDR3 of a variable heavy domain having an amino acid sequence of SEQ ID NO:25, and a variable light domain comprising a vlCDR1, vlCDR2, and vlCDR3 of a variable light domain having an amino acid sequence of SEQ ID NO:29;
e) a variable heavy domain comprising a vhCDR1, vhCDR2, and vhCDR3 of a variable heavy domain having an amino acid sequence of SEQ ID NO:33, and a variable light domain comprising a vlCDR1, vlCDR2, and vlCDR3 of a variable light domain having an amino acid sequence of SEQ ID NO:37;
f) a variable heavy domain comprising a vhCDR1, vhCDR2, and vhCDR3 of a variable heavy domain having an amino acid sequence of SEQ ID NO:41, and a variable light domain comprising a vlCDR1, vlCDR2, and vlCDR3 of a variable light domain having an amino acid sequence of SEQ ID NO:45;
g) a variable heavy domain comprising a vhCDR1, vhCDR2, and vhCDR3 of a variable heavy domain having an amino acid sequence of SEQ ID NO:49, and a variable light domain comprising a vlCDR1, vlCDR2, and vlCDR3 of a variable light domain having an amino acid sequence of SEQ ID NO:53;
h) a variable heavy domain comprising a vhCDR1, vhCDR2, and vhCDR3 of a variable heavy domain having an amino acid sequence of SEQ ID NO:57, and a variable light domain comprising a vlCDR1, vlCDR2, and vlCDR3 of a variable light domain having an amino acid sequence of SEQ ID NO:61;
i) a variable heavy domain comprising a vhCDR1, vhCDR2, and vhCDR3 of a variable heavy domain having an amino acid sequence of SEQ ID NO:65, and a variable light domain comprising a vlCDR1, vlCDR2, and vlCDR3 of a variable light domain having an amino acid sequence of SEQ ID NO:69; and
j) a variable heavy domain comprising a vhCDR1, vhCDR2, and vhCDR3 of a variable heavy domain having an amino acid sequence of SEQ ID NO:73, and a variable light domain comprising a vlCDR1, vlCDR2, and vlCDR3 of a variable light domain having an amino acid sequence of SEQ ID NO:77.

In another aspect, provided herein is an antibody comprising a TNF-like ligand 1A (TL1A) antigen binding domain, the TL1A binding domain comprising a variable heavy domain and a variable light domain selected from the following sets of variable heavy domains and variable light domains:
a) a variable heavy domain having an amino acid sequence that is at least 90% identical to SEQ ID NO:1, and a variable light domain having an amino acid sequence that is at least 90% identical to SEQ ID NO:5;
b) a variable heavy domain having an amino acid sequence that is at least 90% identical to SEQ ID NO:9, and a variable light domain having an amino acid sequence that is at least 90% identical to SEQ ID NO:13;
c) a variable heavy domain having an amino acid sequence that is at least 90% identical to SEQ ID NO:17, and a variable light domain having an amino acid sequence that is at least 90% identical to SEQ ID NO:21;
d) a variable heavy domain having an amino acid sequence that is at least 90% identical to SEQ ID NO:25, and a variable light domain having an amino acid sequence that is at least 90% identical to SEQ ID NO:29;
e) a variable heavy domain having an amino acid sequence that is at least 90% identical to SEQ ID NO:33, and a variable light domain having an amino acid sequence that is at least 90% identical to SEQ ID NO:37;
f) a variable heavy domain having an amino acid sequence that is at least 90% identical to SEQ ID NO:41, and a variable light domain having an amino acid sequence that is at least 90% identical to SEQ ID NO:45;
g) a variable heavy domain having an amino acid sequence that is at least 90% identical to SEQ ID NO:49, and a variable light domain having an amino acid sequence that is at least 90% identical to SEQ ID NO:53;
h) a variable heavy domain having an amino acid sequence that is at least 90% identical to SEQ ID NO:57 and a variable light domain having an amino acid sequence that is at least 90% identical to SEQ ID NO:61;
i) a variable heavy domain having an amino acid sequence that is at least 90% identical to SEQ ID NO:65, and a variable light domain having an amino acid sequence that is at least 90% identical to SEQ ID NO:69; and
j) a variable heavy domain having an amino acid sequence that is at least 90% identical to SEQ ID NO:73, and a variable light domain having an amino acid sequence that is at least 90% identical to SEQ ID NO:77.

In yet another aspect, provided herein is an antibody comprising a TNF-like ligand 1A (TL1A) antigen binding domain, the TL1A binding domain comprising a variable heavy domain and a variable light domain selected from the following sets of variable heavy domains and variable light domains:
a) a variable heavy domain having an amino acid sequence of SEQ ID NO:1, and a variable light domain having an amino acid sequence of SEQ ID NO: 5;
b) a variable heavy domain having an amino acid sequence of SEQ ID NO:9, and a variable light domain having an amino acid sequence of SEQ ID NO:13;
c) a variable heavy domain having an amino acid sequence of SEQ ID NO:17, and a variable light domain having an amino acid sequence of SEQ ID NO:21;
d) a variable heavy domain having an amino acid sequence of SEQ ID NO:25, and a variable light domain having an amino acid sequence of SEQ ID NO:29;
e) a variable heavy domain having an amino acid sequence of SEQ ID NO:33, and a variable light domain having an amino acid sequence of SEQ ID NO:37;
f) a variable heavy domain having an amino acid sequence of SEQ ID NO:41, and a variable light domain having an amino acid sequence of SEQ ID NO:45;
g) a variable heavy domain having an amino acid sequence of SEQ ID NO:49, and a variable light domain having an amino acid sequence of SEQ ID NO:53;
h) a variable heavy domain having an amino acid sequence of SEQ ID NO:57 and a variable light domain having an amino acid sequence of SEQ ID NO:61;
i) a variable heavy domain having an amino acid sequence of SEQ ID NO:65, and a variable light domain having an amino acid sequence of SEQ ID NO:69; and
j) a variable heavy domain having an amino acid sequence of SEQ ID NO:73, and a variable light domain having an amino acid sequence of SEQ ID NO:77.

In some embodiments, the antibody further comprises a dimeric variant IgG Fc domain comprising a first monomeric variant IgG Fc domain and a second monomeric variant IgG Fc domain, wherein each of the first monomeric variant IgG Fc domain and the second monomeric variant IgG Fc domain each comprises amino acid substitutions E233P/L234V/L235A/G236del/S267K, and wherein numbering is according to EU numbering.

In some embodiments, the antibody further comprises a dimeric variant IgG Fc domain comprising a first monomeric variant IgG Fc domain and a second monomeric variant IgG Fc domain, wherein each of the first monomeric variant IgG Fc domain and the second monomeric variant IgG Fc domain each comprises amino acid substitutions L234A/L235A/P329A, and wherein numbering is according to EU numbering.

In some embodiments, the antibody further comprises a dimeric variant IgG Fc domain comprising a first monomeric variant IgG Fc domain and a second monomeric variant IgG Fc domain, wherein each of the first monomeric variant IgG Fc domain and the second monomeric variant IgG Fc domain each comprises amino acid substitutions M428L/N434S, and wherein numbering is according to EU numbering.

In exemplary embodiments, each of the first monomeric variant IgG Fc domain and second monomeric variant IgG Fc domain are variants of a monomeric wild-type human IgG1 Fc domain.

In another aspect, provided herein is a TNF-like ligand 1A (TL1A) antigen binding domain, the TL1A binding domain comprising a variable light domain and a variable heavy domain selected from the following sets of variable light domains and variable heavy domains:
a) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
b) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
c) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
d) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
e) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
f) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
g) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
h) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
i) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
j) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80.

In another aspect, provided herein is a TNF-like ligand 1A (TL1A) antigen binding domain, the TL1A binding domain comprising a variable heavy domain and a variable light domain selected from the following sets of variable heavy domains and variable light domains:
a) a variable heavy domain comprising a vhCDR1, vhCDR2, and vhCDR3 of a variable heavy domain having an amino acid sequence of SEQ ID NO:1, and a variable light domain comprising a vlCDR1, vlCDR2, and vlCDR3 of a variable light domain having an amino acid sequence of SEQ ID NO:5;
b) a variable heavy domain comprising a vhCDR1, vhCDR2, and vhCDR3 of a variable heavy domain having an amino acid sequence of SEQ ID NO:9, and a variable light domain comprising a vlCDR1, vlCDR2, and vlCDR3 of a variable light domain having an amino acid sequence of SEQ ID NO:13;
c) a variable heavy domain comprising a vhCDR1, vhCDR2, and vhCDR3 of a variable heavy domain having an amino acid sequence of SEQ ID NO:17, and a variable light domain comprising a vlCDR1, vlCDR2, and vlCDR3 of a variable light domain having an amino acid sequence of SEQ ID NO:21;
d) a variable heavy domain comprising a vhCDR1, vhCDR2, and vhCDR3 of a variable heavy domain having an amino acid sequence of SEQ ID NO:25, and a variable light domain comprising a vlCDR1, vlCDR2, and vlCDR3 of a variable light domain having an amino acid sequence of SEQ ID NO:29;
e) a variable heavy domain comprising a vhCDR1, vhCDR2, and vhCDR3 of a variable heavy domain having an amino acid sequence of SEQ ID NO:33, and a variable light domain comprising a vlCDR1, vlCDR2, and vlCDR3 of a variable light domain having an amino acid sequence of SEQ ID NO:37;
f) a variable heavy domain comprising a vhCDR1, vhCDR2, and vhCDR3 of a variable heavy domain having an amino acid sequence of SEQ ID NO:41, and a variable light domain comprising a vlCDR1, vlCDR2, and vlCDR3 of a variable light domain having an amino acid sequence of SEQ ID NO:45;
g) a variable heavy domain comprising a vhCDR1, vhCDR2, and vhCDR3 of a variable heavy domain having an amino acid sequence of SEQ ID NO:49, and a variable light domain comprising a vlCDR1, vlCDR2, and vlCDR3 of a variable light domain having an amino acid sequence of SEQ ID NO:53;
h) a variable heavy domain comprising a vhCDR1, vhCDR2, and vhCDR3 of a variable heavy domain having an amino acid sequence of SEQ ID NO:57, and a variable light domain comprising a vlCDR1, vlCDR2, and vlCDR3 of a variable light domain having an amino acid sequence of SEQ ID NO:61;
i) a variable heavy domain comprising a vhCDR1, vhCDR2, and vhCDR3 of a variable heavy domain having an amino acid sequence of SEQ ID NO:65, and a variable light domain comprising a vlCDR1, vlCDR2, and vlCDR3 of a variable light domain having an amino acid sequence of SEQ ID NO:69; and
j) a variable heavy domain comprising a vhCDR1, vhCDR2, and vhCDR3 of a variable heavy domain having an amino acid sequence of SEQ ID NO:73, and a variable light domain comprising a vlCDR1, vlCDR2, and vlCDR3 of a variable light domain having an amino acid sequence of SEQ ID NO:77.

In yet another aspect, provided herein is a TNF-like ligand 1A (TL1A) antigen binding domain, the TL1A binding domain comprising a variable heavy domain and a variable light domain selected from the following sets of variable heavy domains and variable light domains:
a) a variable heavy domain having an amino acid sequence that is at least 90% identical to SEQ ID NO:1, and a variable light domain having an amino acid sequence that is at least 90% identical to SEQ ID NO:5;
b) a variable heavy domain having an amino acid sequence that is at least 90% identical to SEQ ID NO:9, and a variable light domain having an amino acid sequence that is at least 90% identical to SEQ ID NO:13;
c) a variable heavy domain having an amino acid sequence that is at least 90% identical to SEQ ID NO:17, and a variable light domain having an amino acid sequence that is at least 90% identical to SEQ ID NO:21;
d) a variable heavy domain having an amino acid sequence that is at least 90% identical to SEQ ID NO:25, and a variable light domain having an amino acid sequence that is at least 90% identical to SEQ ID NO:29;
e) a variable heavy domain having an amino acid sequence that is at least 90% identical to SEQ ID NO:33, and a variable light domain having an amino acid sequence that is at least 90% identical to SEQ ID NO:37;
f) a variable heavy domain having an amino acid sequence that is at least 90% identical to SEQ ID NO:41, and a variable light domain having an amino acid sequence that is at least 90% identical to SEQ ID NO:45;
g) a variable heavy domain having an amino acid sequence that is at least 90% identical to SEQ ID NO:49, and a variable light domain having an amino acid sequence that is at least 90% identical to SEQ ID NO:53;
h) a variable heavy domain having an amino acid sequence that is at least 90% identical to SEQ ID NO:57 and a variable light domain having an amino acid sequence that is at least 90% identical to SEQ ID NO:61;
i) a variable heavy domain having an amino acid sequence that is at least 90% identical to SEQ ID NO:65, and a variable light domain having an amino acid sequence that is at least 90% identical to SEQ ID NO:69; and
j) a variable heavy domain having an amino acid sequence that is at least 90% identical to SEQ ID NO:73, and a variable light domain having an amino acid sequence that is at least 90% identical to SEQ ID NO:77.

In one aspect, provided herein is an TNF-like ligand 1A (TL1A) antigen binding domain, the TL1A binding domain comprising a variable heavy domain and a variable light domain selected from the following sets of variable heavy domains and variable light domains:
a) a variable heavy domain having an amino acid sequence of SEQ ID NO:1, and a variable light domain having an amino acid sequence of SEQ ID NO: 5;
b) a variable heavy domain having an amino acid sequence of SEQ ID NO:9, and a variable light domain having an amino acid sequence of SEQ ID NO:13;
c) a variable heavy domain having an amino acid sequence of SEQ ID NO:17, and a variable light domain having an amino acid sequence of SEQ ID NO:21;
d) a variable heavy domain having an amino acid sequence of SEQ ID NO:25, and a variable light domain having an amino acid sequence of SEQ ID NO:29;
e) a variable heavy domain having an amino acid sequence of SEQ ID NO:33, and a variable light domain having an amino acid sequence of SEQ ID NO:37;
f) a variable heavy domain having an amino acid sequence of SEQ ID NO:41, and a variable light domain having an amino acid sequence of SEQ ID NO:45;
g) a variable heavy domain having an amino acid sequence of SEQ ID NO:49, and a variable light domain having an amino acid sequence of SEQ ID NO:53;
h) a variable heavy domain having an amino acid sequence of SEQ ID NO:57 and a variable light domain having an amino acid sequence of SEQ ID NO:61;
i) a variable heavy domain having an amino acid sequence of SEQ ID NO:65, and a variable light domain having an amino acid sequence of SEQ ID NO:69; and
j) a variable heavy domain having an amino acid sequence of SEQ ID NO:73, and a variable light domain having an amino acid sequence of SEQ ID NO:77.

In another aspect, provided herein is an anti-TL1A antibody comprising a heavy chain having an amino acid sequence of SEQ ID NO: 123, and a light chain having an amino acid sequence of SEQ ID NO: 124.

In another aspect, provided herein is a method of inhibiting or reducing TL1A-mediated cell apoptosis in a subject in need thereof, wherein the method comprises administrating to the subject an anti-TL1A antibody provided herein.

In yet another aspect, provided herein is a method of inhibiting or reducing TL1A-mediated NFkB signaling in a subject in need thereof, wherein the method comprises administrating to the subject an anti-TL1A antibody provided herein.

In another aspect, provided herein is a method of inhibiting or reducing TL1A-induced cytokine secretion in a subject in need thereof, wherein the method comprises administrating to the subject an anti-TL1A antibody provided herein. In some embodiments, the cytokine is selected from the following cytokines: interferon-γ (IFN-γ), interleukin-1β (IL-1β); tumor necrosis factor-α (TNF-α); transforming growth factor-β (TGF-β); and granulocyte-macrophage colony stimulating factor (GM-CSF).

In some embodiments of the aforementioned methods, the subject has a TL1A-associated disease (e.g., an inflammatory bowel disease). In some embodiments, the TL1A-associated disease is one of the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis.

In another aspect, provided herein is a method for treating a TL1A-associated disease (e.g., an inflammatory bowel disease), wherein the method comprises administering to the subject in need thereof an anti-TL1A antibody provided herein. In some embodiments, the TL1A-associated disease is one of the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis.

In some embodiments of the methods provided herein, the subject is a human.

In another aspect, provided herein an anti-TL1A antibody for use in inhibiting or reducing TL1A-mediated cell apoptosis, wherein the antibody is an anti-TL1A antibody provided herein. In some embodiments, the TL1A-mediated cell apoptosis is inhibited or reduced in a subject in need thereof. In some embodiments, the subject is a human. In some embodiments, the subject has a TL1A-associated disease. In some embodiments, the disease is an inflammatory bowel disease. In some embodiments, the TL1A-associated disease is selected from the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis.

In another aspect, provided herein is a use of an anti-TL1A provided herein for the manufacture of a medicament for inhibiting or reducing TL1A-mediated cell apoptosis in a subject in need thereof. In some embodiments, the subject is a human. In some embodiments, the subject has a TL1A-associated disease. In some embodiments, the disease is an inflammatory bowel disease. In some embodiments, the TL1A-associated disease is selected from the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis.

In another aspect, provided herein an anti-TL1A antibody for use in inhibiting or reducing TL1A-mediated NFkB signaling, wherein the antibody is an anti-TL1A antibody provided herein. In some embodiments, the TL1A-mediated NFkB signaling is inhibited or reduced in a subject in need thereof. In some embodiments, the subject is a human. In some embodiments, the subject has a TL1A-associated disease. In some embodiments, the disease is an inflammatory bowel disease. In some embodiments, the TL1A-associated disease is selected from the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis.

In another aspect, provided herein is a use of an anti-TL1A provided herein for the manufacture of a medicament for inhibiting or reducing TL1A-mediated NFkB signaling in a subject in need thereof. In some embodiments, the subject is a human. In some embodiments, the disease is an inflammatory bowel disease. In some embodiments, the TL1A-associated disease is selected from the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis. [0028]n another aspect, provided herein an anti-TL1A antibody for use in inhibiting or reducing TL1A-induced cytokine secretion, wherein the antibody is an anti-TL1A antibody provided herein. In some embodiments, the TL1A-induced cytokine secretion is inhibited or reduced in a subject in need thereof. In some embodiments, the subject is a human. In some embodiments, the subject has a TL1A-associated disease. In some embodiments, the disease is an inflammatory bowel disease. In some embodiments, the TL1A-associated disease is selected from the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis. In some embodiments, the cytokine is selected from one of the following: interferon-γ (IFN-γ); interleukin-1β (IL-1β); tumor necrosis factor-α (TNF-α); transforming growth factor-β (TGF-β);
granulocyte-macrophage colony stimulating factor (GM-CSF).

In another aspect, provided herein is a use of an anti-TL1A provided herein for the manufacture of a medicament for inhibiting or reducing TL1A-induced cytokine secretion in a subject in need thereof. In some embodiments, the subject is a human. In some embodiments, the subject has a TL1A-associated disease. In some embodiments, the disease is an inflammatory bowel disease. In some embodiments, the TL1A-associated disease is selected from the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis. In some embodiments, the cytokine is selected from one of the following: interferon-γ (IFN-γ); interleukin-1β (IL-1β); tumor necrosis factor-α (TNF-α); transforming growth factor-β (TGF-β); granulocyte-macrophage colony stimulating factor (GM-CSF).

In another aspect, provided herein is an anti-TL1A antibody for use in the treatment of a TL1A-associated disease in a subject in need thereof. In some embodiments, the subject is a human. In some embodiments, the subject has a TL1A-associated disease. In some embodiments, the disease is an inflammatory bowel disease. In some embodiments, the TL1A-associated disease is selected from the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis. In some embodiments, the cytokine is selected from one of the following: interferon-γ (IFN-γ); interleukin-1β (IL-1β); tumor necrosis factor-α (TNF-α); transforming growth factor-β (TGF-β); granulocyte-macrophage colony stimulating factor (GM-CSF).

In another aspect, provided herein is a use of an anti-TL1A antibody provided herein for the manufacture of a medicament for the treatment of a TL1A-associated disease. In some embodiments, the subject is a human. In some embodiments, the subject has a TL1A-associated disease. In some embodiments, the disease is an inflammatory bowel disease. In some embodiments, the TL1A-associated disease is selected from the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis. In some embodiments, the cytokine is selected from one of the following: interferon-γ (IFN-γ); interleukin-1β (IL-1β); tumor necrosis factor-α (TNF-α); transforming growth factor-β (TGF-β); granulocyte-macrophage colony stimulating factor (GM-CSF).

### BRIEF DESCRIPTION OF THE DRAWINGS

FIG. 1 depicts the sequences for human, mouse, and rat TL1A (TNF-like ligand 1A). Such TL1A sequences are useful for the development of TL1A antigen binding domains. TL1A may also be referred to as tumor necrosis factor superfamily 15 (TNFS15) or vascular endothelial growth inhibitor (VEGI). Unless otherwise noted, in general references to TL1a herein are directed to the human TL1a protein.

FIG. 2 depicts useful ablation variants that ablate FcγR binding (also referred to as “knockouts” or “KO” variants). In some embodiments, such ablation variants are included in the Fc domain of both monomers of the subject antibody described herein.

FIG. 3 depicts the sequences of several useful backbones based on human IgG1, including the subject anti-TL1A antibodies and related methods disclosed herein. Fc backbone 1 is based on human IgG1 (356E/358M allotype) and includes the E233P/L234V/L235A/G236del/S267K ablation variant on both chains. Fc backbone 2 is based on human IgG1 (356E/358M allotype) and includes the E233P/L234V/L235A/G236del/S267K ablation variant on both chains and M428L/N434S Xtend variants on both chains. Fc backbone 3 is based on human IgG1 (356E/358M allotype) and includes the L234A/L235A/P329A ablation variant on both chains and M428L/N434S Xtend variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity. Additionally, C-terminal glycine and/or lysine deletion may occur naturally for example during production and storage.

FIG. 4 depicts sequences for “CH1” that find use in embodiments of anti-TL1A antibodies.

FIG. 5 depicts sequences for “hinge” that find use in embodiments of anti-TL1A antibodies.

FIG. 6 depicts the constant domain of the cognate light chains that find use in embodiments of anti-TL1A antibodies.

FIG. 7 depicts Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to:

FIG. 8 depicts the sequence for exemplary rat anti-TL1A antibody XENP47498, having a TL1A binding domain designated 70416_041[TL1A]_H0L0. CDRs are underlined and slashes indicate the borders of variable regions.

FIGS. 9A-9D depict the variable heavy and variable light chain sequences for TL1A binding domains which find use in the invention, including any of the subject TL1A antibodies and related methods disclosed herein. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems.

FIGS. 10A-10C depict amino acid sequences for exemplary TL1A antibodies having the PVA_/S267K Fc ablation variants. CDRs are underlined and slashes indicate the border(s) of variable regions.

FIGS. 11A-11C depict amino acid sequences for exemplary TL1A antibodies having the M428L/N434S Xtend variants in addition to the PVA_/S267K Fc ablation variants. CDRs are underlined and slashes indicate the border(s) of variable regions.

FIGS. 12A-12C depict the sequence for exemplary TL1A mAbs having the L234A/L235A/P329A Fc ablation variants and the M428L/N434S Xtend variants. CDRs are underlined and slashes indicate the borders of variable regions.

FIG. 13 depicts the monovalent binding affinity data for anti-TL1A Fab to scTL1A.

FIG. 14 depicts TL1A induced caspase activity in TF-1 cells and resulting IC50 values.

FIG. 15 depicts the pharmacokinetic profile in huFcRn mice of anti-TL1A mAbs of the invention.

FIGS. 16A-16D depict a summary of the results of studies to assess the binding of exemplary subject anti-TL1A antibodies to TL1A. FIG. 16A depicts binding of anti-TL1A mAbs to TL1A transiently expressed on CHO cells. FIG. 16B depicts the binding of XmAb942 to soluble recombinant human TL1A trimer via surface plasmon resonance. FIG. 16C depicts the binding of anti-TL1A Fabs (200 nM) to immobilized TL1A monomer or trimer at 25° C. via biolayer interferometry. FIG. 16D summarizes the binding data of XmAb942 relative to Comparator 1 and Comparator 2.

FIG. 17 depicts the results of a study showing inhibition of TL1A-induced NFKB signaling by anti-TL1A mAbs.

FIG. 18 depicts the results of a study inhibition of TL1A-induced IFNγ release by anti-TL1A mAbs.

FIG. 19 depicts the improved half-life of XmAb942 in monkeys compared with first generation anti-TL1A mAbs Comparator 1 and Comparator 2.

FIGS. 20A and 20B depict the thermostability of anti-TL1A Fab domains using differential scanning calorimetry.

FIG. 21 depicts Quantitative Systems Pharmacology (QSP) model simulations based on Xmab942 in vitro and NHP data (ti/2 of 23 days in NHPs), and competitor human PK/PD data. These simulations predict best-in-class TL1A suppression.

FIG. 22 depicts BLI binding data from an Octet RH96 which show that XmAb942 is specific for TL1A binding and does not bind other similar TNFα superfamily members, while Comparator 3 shows some off-target binding to LIGHT.

FIG. 23 depicts binding of XmAb942 and Comparators to human FcyRs. XmAb942 was the only sample completely negative for binding to 1 μM concentrations of any of the receptors tested.

FIGS. 24A-24D depict the variable heavy and variable light chain sequences for TL1A binding domains of relevant comparator anti-TL1A antibodies. As noted herein and is true for every sequence herein containing CDRs, the exact identification of the CDR locations may be different depending on the numbering used as is shown in Table 2, and thus included herein are not only the CDRs that are underlined but also CDRs included within the VH and VL domains using other numbering systems.

FIG. 25 depicts the amino acid sequence for anti-TL1A antibodies having the variable heavy and variable light domains of Comparator 1 with differing Fc domains.

FIG. 26 depicts the amino acid sequence for anti-TL1A antibodies having the variable heavy and variable light domains of Comparator 2 with differing Fe domains.

FIGS. 27A and 27B depict the amino acid sequence for anti-TL1A antibodies having the variable heavy and variable light domains of Comparator 3-1, Comparator 3-2, or Comparator 3-3 with differing Fc domains.

FIGS. 28A and 28B depict the amino acid sequence for anti-TL1A antibodies having the variable heavy and variable light domains of Comparator 4, or Comparator 5 with differing Fc domains.

FIG. 29 depicts a summary of the results of additional studies to assess the binding of exemplary subject anti-TL1A antibodies to TL1A.

FIG. 30 depicts the thermostability of anti-TL1A Fab domains.

FIG. 31 depicts the PK and PD profiles obtained from Quantitative Systems Pharmacology (QSP) modelling for XmAb942 dosing regimens, wherein induction doses are administered every four weeks (qw4, iv) at weeks 0, 4, and 8 at three different doses (D1, D2, and D3), followed by maintenance doses once every eight weeks (qw8, sc) at dose Dm.

FIG. 32 depicts the XmAb942 QSP modeling results as compared to the results of two comparator anti-TL1A antibodies (“Comparator 1” and “Comparator 2”). The QSP modeling results in FIGS. 3 and 4 were based on human PK and PD data for the comparators and non-clinical data for XmAb942.

FIG. 33 provides a clinical development plan for XmAb942.

FIG. 34 provides the results of human PK data for XmAb942 administered intravenously (left) and subcutaneously (right).

FIG. 35 provides the summary of a study on the pharmacodynamic effects of intravenous and subcutaneous administration of XmAb942 on human patients.

FIG. 36 provides an overview of a human Phase 2b study of XmAb942 for the treatment of ulcerative colitis.

FIG. 37 is a half-life study, showing that XmAb942 demonstrates increased half-life in monkeys compared with first-generation anti-TL1A antibodies.

FIG. 38 provides additional results of human PK data for XmAb942 at three different doses.

FIGS. 39A and 39B provides the summary of a study on the pharmacodynamic effects of intravenous and subcutaneous administration of XmAb942 on human patients at three different doses.

FIG. 40 depicts the PD profiles obtained from modelling for XmAb942 dosing regimens at three different doses.

### DETAILED DESCRIPTION

### I. Overview

Provided herein are novel TNF-Like Ligand 1A (TL1A) binding domains and antibodies that include such binding domains. The TL1A binding domains and antibodies provided herein are useful, for example, in the treatment of TL1A-associated diseases. In exemplary embodiments, the novel antibodies also include Fc variants to extend serum half-life in patients.

### II. Definitions

In order that the application may be more completely understood, several definitions are set forth below. Such definitions are meant to encompass grammatical equivalents.

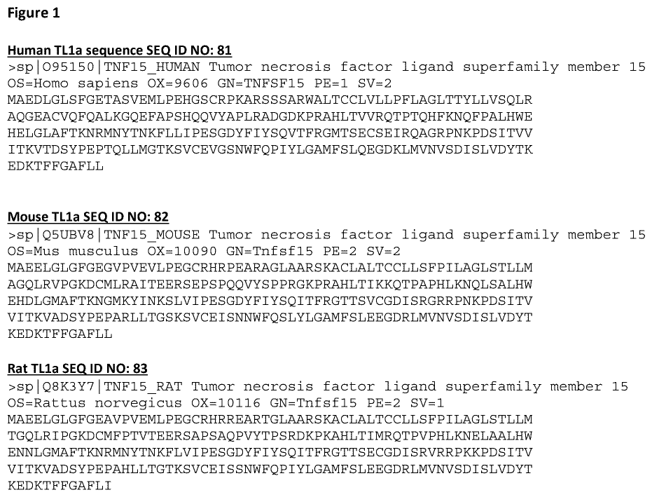

As used herein, “tumor necrosis factor-like cytokine 1A,” “TL1A,” “TNFSF15,” “vascular endothelial growth inhibitor,” “TNF super family member 15,” all refer to a member of the tumor necrosis family that is capable of binding to the functional receptor Death Receptor 3 (DR3) on T cells. Exemplary sequences of TL1A are depicted in FIG. 1. TL1A can activate both the NF-κB and MAPK signaling pathways, and acts as an autocrine factor to induce apoptosis in endothelial cells. TL1A is also found to inhibit endothelial cell proliferation, and thus is believed to function as an angiogenesis inhibitor. In humans, TL1A is encoded by the TNFSF15 gene, and several TNFSF15 SNPs have been found to be associated with inflammatory bowel disease.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

As used herein, the term “antibody” refers to traditional immunoglobulin (Ig) antibodies unless stated specifically otherwise.

Traditional immunoglobulin (Ig) antibodies are “Y” shaped tetramers. Each tetramer is typically composed of two identical pairs of polypeptide chains, each pair having one “light chain” monomer and one “heavy chain” monomer.

An antibody heavy chain typically includes a variable heavy (VH) domain (also referred to as “a heavy chain variable domain”), which includes vhCDR1-3, and an Fc domain, which includes a CH2-CH3 monomer. In some embodiments, an antibody heavy chain includes a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention, including any of the subject TL1A antibodies and methods disclosed herein, are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

By “Fc” or “Fc region” or “Fc domain” as used herein is meant the polypeptide comprising the constant region of an antibody, in some instances, excluding all of the first constant region immunoglobulin domain (e.g., CHI) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). In some embodiments, the Fc domain is from IgG1, IgG2, IgG3 or IgG4, with IgG1 hinge-CH2-CH3 and IgG4 hinge-CH2-CH3 finding particular use in many embodiments. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” or “constant heavy domain” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus, for IgG the antibody hinge is herein defined to include positions 216 (E216 in IgG1) to 230 (P230 in IgG1), wherein the numbering is according to the EU index as in Kabat.

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference. Other numbering conventions are available in the art and those skilled in the art would readily be able to determine the exact numbering and placement in those other numbering convention systems based on what's described herein.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL) (also referred to as “light chain variable domain”), which includes light chain CDRs vlCDR1-3, and a constant light chain region or light chain constant region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of antibody sequences, specifically binds a target antigen (e.g., human TL1A) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 variable light CDRs. The CDRs are present in the variable heavy domain (vhCDR1-3) and variable light domain (vlCDR1-3). The variable heavy domain and variable light domain form an Fv region.

Typically, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, i.e., a heavy and light chain, respectively.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1):55-77 (2003):

TABLE 2

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)). Those skilled in the art would readily be able to determine the exact numbering and placement of the CDRs in other numbering systems described herein and known in the art.

The CDRs contribute to the formation of the antigen-binding, or more specifically, epitope binding site of the antibody. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention, including any of the subject TL1A antibodies and methods disclosed herein, not only includes the enumerated antigen binding domains and antibodies herein, but those that compete for binding with the epitopes bound by the enumerated antigen binding domains.

The six CDRs of the subject antibodies are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain).

By “variable region” or “variable domain” as used herein is meant the region of an immunoglobulin that comprises one or more Ig domains substantially encoded by any of the Vx, VW, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of an antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution M428L refers to a variant polypeptide, in this case an Fc variant, in which the methionine at position 272 is replaced with leucine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below.

As described below, in some embodiments the parent polypeptide, for example an Fc parent polypeptide, is a human wild-type sequence, such as the heavy constant domain or Fc region from IgG1 or IgG2, although human sequences with variants can also serve as “parent polypeptides”, for example the IgG1/2 hybrid of US Publication 2006/0134105 can be included. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1 or IgG2.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fc domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). Those skilled in the art would readily be able to determine the corresponding positions in other numbering systems described herein and known in the art. The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides, and peptides. In addition, polypeptides that make up the antibodies of the invention, including any of the subject TL1A antibodies disclosed herein, may include synthetic derivatization of one or more side chains or termini, glycosylation, PEGylation, circular permutation, cyclization, linkers to other molecules, fusion to proteins or protein domains, and addition of peptide tags or labels.

By “non-naturally occurring modification” as used herein is meant an amino acid modification that is not isotypic. For example, because none of the known human IgGs comprise a serine at position 434, the substitution 434S in IgG1 or IgG2 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “wild type” or “WT” or “wild-type” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention, including any of the subject TL1A antibodies disclosed herein, are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody that is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction using an assay described herein or known in the art. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope. A suitable control molecule is described herein including the Example sections and known in the art.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### III. TL1A Binding Domains

In one aspect, provided herein are TL1A antigen binding domains (ABDs) that bind TL1A, and constructs (e.g., antibodies and fusion proteins) that include such TL1A antigen binding domains. The TL1AABDs provided herein generally include a variable heavy domain (VH) and a variable light domain (VL). In exemplary embodiments, the TL1A is capable of binding to human TL1A (see FIG. 1).

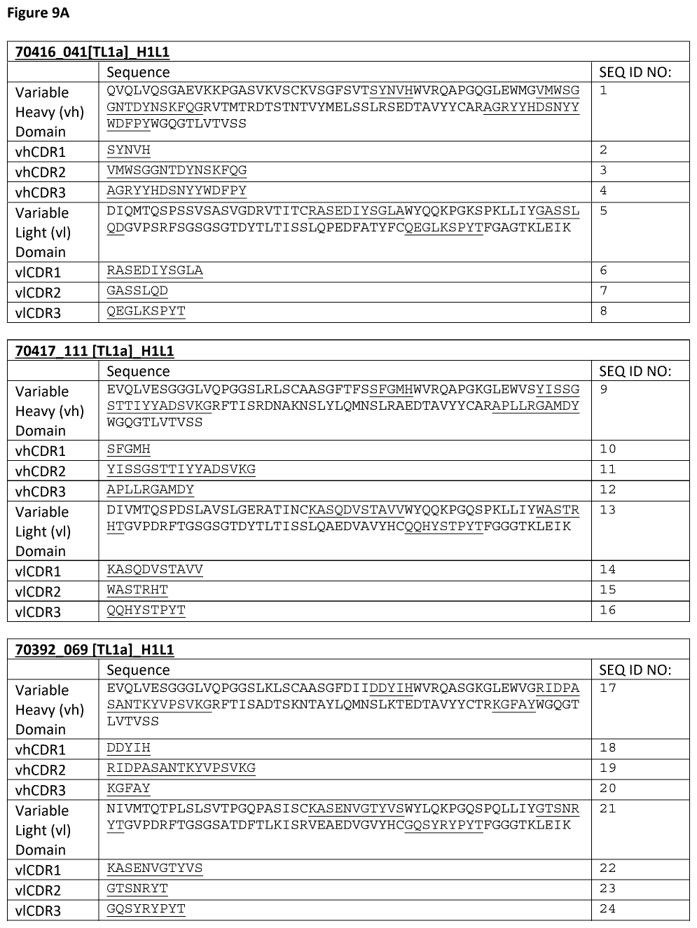

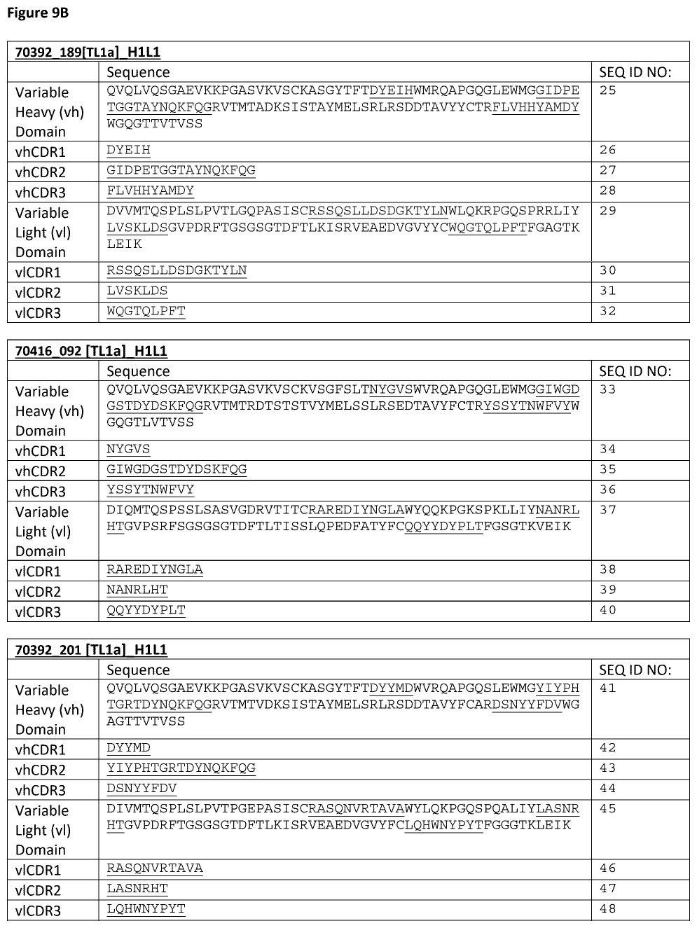

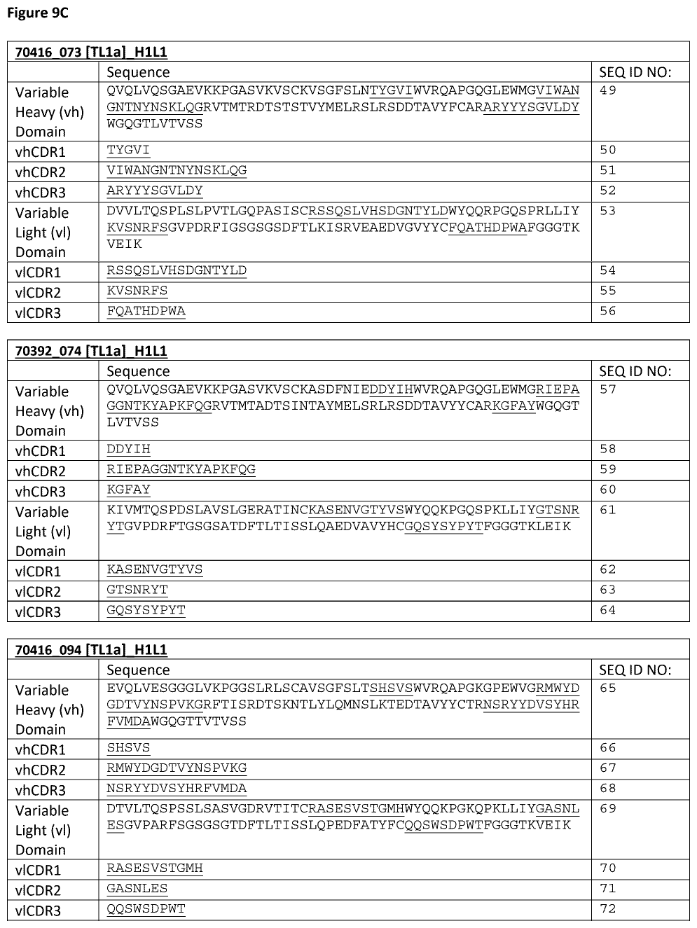

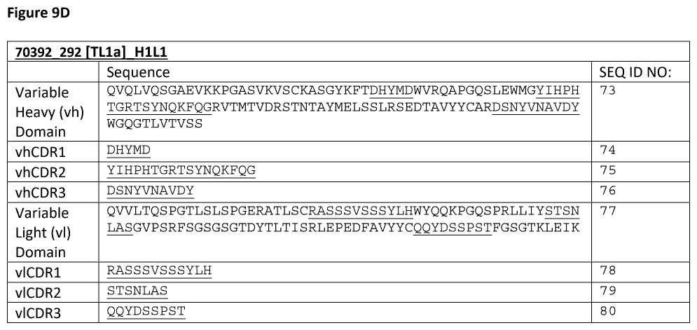

As will be appreciated by those in the art, suitable TL1A binding domains can comprise a set of 6 CDRs as depicted in the figures, either as they are underlined or, in the case where a different numbering scheme is used as described herein and as shown in Table 2, as the CDRs that are identified using other alignments within the variable heavy (VH) domain and variable light domain (VL) sequences of those depicted in FIG. 9. Suitable TL1AABDs can also include the entire VH and VL sequences as depicted in these sequences and figures, used as scFvs or as Fabs.

In one embodiment, the TL1A antigen binding domain includes the 6 CDRs (i.e., vhCDR1-3 and vlCDR1-3) of any of the TL1A binding domains described herein, including the figures. In some embodiments, the TL1AABD is one of the following TL1A ABDs: 70416_041[TL1a]_H1L1, 70417_111 [TL1a]_H1L1, 70392_069 [TL1a]_H1L1, 70392189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (FIG. 9).

In addition to the parental CDR sets disclosed in the figures that form the TL1AABDs (e.g., FIG. 9), provided herein are variant TL1AABDS having CDRs that include at least one modification of the TL1AABD CDRs disclosed herein (e.g., FIG. 9). In one embodiment, the TL1AABD includes a set of 6 CDRs with 1, 2, 3, 4, 5, 6, 7, 8, 9, 10 amino acid modifications as compared to the 6 CDRs of a TL1AABD as described herein, including the figures. In exemplary embodiments, the TL1AABD includes a set of 6 CDRs with 1, 2, 3, 4, 5, 6, 7, 8, 9, 10 amino acid modifications as compared to the 6 CDRs of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (FIG. 9). In certain embodiments, the TL1A ABD is capable of binding TL1A antigen, as measured by at least one of a Biacore, surface plasmon resonance (SPR) and/or BLI (biolayer interferometry, e.g., Octet assay) assay, with the latter finding particular use in many embodiments. In particular embodiments, the TL1A ABD is capable of binding human TL1A antigen (see FIG. 1).

In some embodiments, the TL1AABD includes 6 CDRs that are at least 90, 95, 97, 98 or 99% identical to the 6 CDRs of a TL1AABD as described herein, including the figures. In exemplary embodiments, the TL1A ABD includes 6 CDRs that are at least 90, 95, 97, 98 or 99% identical to the 6 CDRs of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (FIG. 9). In certain embodiments, the TL1AABD is capable of binding TL1A antigen, as measured by at least one of a Biacore, surface plasmon resonance (SPR) and/or BLI (biolayer interferometry, e.g., Octet assay) assay, with the latter finding particular use in many embodiments. In particular embodiments, the TL1AABD is capable of binding human TL1A antigen (see FIG. 1).

In another exemplary embodiment, the TL1AABD include the variable heavy (VH) domain and variable light (VL) domain of any one of the TL1A ABDs described herein, including the figures. In exemplary embodiments, the TL1AABD is one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (FIG. 9).

In addition to the parental TL1A variable heavy and variable light domains disclosed herein, provided herein are TL1AABDs that include a variable heavy domain and/or a variable light domain that are variants of a TL1AABD VH and VL domain disclosed herein. In one embodiment, the variant VH domain and/or VL domain has from 1, 2, 3, 4, 5, 6, 7, 8, 9 or 10 amino acid changes from a VH and/or VL domain of a TL1AABD described herein, including the figures. In some embodiments, the variant VH domain and/or VL domain has includes 1, 2, 3, 4, 5, 6, 7, 8, 9 or 10 amino acid changes in a framework region (e.g., FR1, FR2, FR3, or FR4) from a VH and/or VL domain of a TL1AABD described herein, including the figures. In exemplary embodiments, the variant VH domain and/or VL domain has from 1, 2, 3, 4, 5, 6, 7, 8, 9 or 10 amino acid changes from a VH and/or VL domain of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (FIG. 9). In exemplary embodiments, the variant VH domain and/or VL domain has from 1, 2, 3, 4, 5, 6, 7, 8, 9 or 10 amino acid changes in a framework region (e.g., FR1, FR2, FR3, or FR4) of the VH and/or VL domain of one of the following TL1AABDs: 70416_041[TL1a]_H1L1, 70417111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (FIG. 9). In certain embodiments, the TL1AABD is capable of binding to TL1A, as measured at least one of a Biacore, surface plasmon resonance (SPR) and/or BLI (biolayer interferometry, e.g., Octet assay) assay, with the latter finding particular use in many embodiments. In particular embodiments, the TL1AABD is capable of binding human TL1A antigen (see FIG. 1).

In one embodiment, the variant VH and/or VL domain is at least 90, 95, 97, 98 or 99% identical to the VH and/or VL of a TL1AABD as described herein, including the figures. In exemplary embodiments, the variant VH and/or VL domain is at least 90, 95, 97, 98 or 99% identical to the VH and/or VL of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (FIG. 9). In certain embodiments, the TL1AABD is capable of binding to TL1A, as measured by at least one of a Biacore, surface plasmon resonance (SPR) and/or BLI (biolayer interferometry, e.g., Octet assay) assay, with the latter finding particular use in many embodiments. In particular embodiments, the TL1AABD is capable of binding human TL1A antigen (see FIG. 1).

### IV. Antibodies

In one aspect, provided herein are anti-TL1A antibodies that include any of the TL1A antigen binding domains described herein. In some embodiments, the anti-TL1A antibody includes 1, 2, 3, 4, 5, 6, 7, 8, 9 or 10 of the TL1AABDs described herein. In some embodiments, the anti-TL1A antibody is a monospecific bivalent antibody.

The antibodies provided herein include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

In some embodiments, the subject antibody includes one or more of the TL1A ABDs provided herein. In some embodiments, the antibody includes one TL1AABD. In other embodiments, the antibody includes two TL1AABDs. In exemplary embodiments, the TL1A ABD includes the variable heavy domain and variable light domain of one of the following TL1AABDs: 70416_041[TL1a]_H1L1, 70417_111 [TL1a]_H1L1, 70392_069 [TL1a]_H1L1, 70392_189 [TL1a]_H1L1, 70416_092 [TL1a]_H1L1, 70392_201 [TL1a]_H1L1, 70416_073 [TL1a]_H1L1, 70392_074 [TL1a]_H1L1, 70416_094 [TL1a]_H1L1, 70392_292 [TL1a]_H1L1 (FIG. 9).

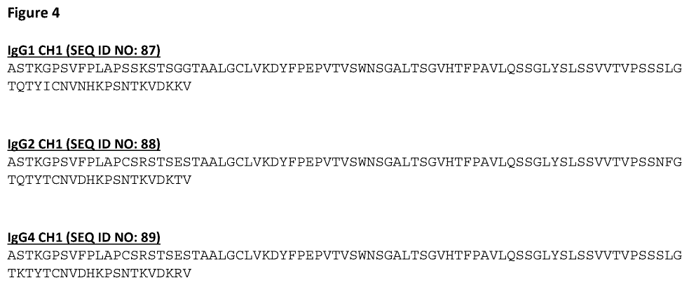

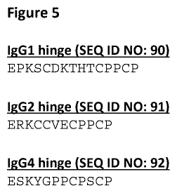

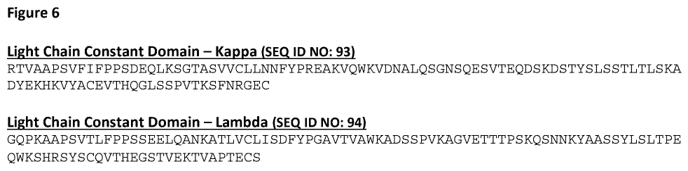

Exemplary sequence of domains (e.g., CH1, hinge, and CL domains) for use the subject antibodies provided herein are depicted in FIGS. 4-6.

In certain embodiments, the anti-TL1A antibody is a monospecific anti-TL1A antibody that includes a TL1A antigen binding domains described herein (e.g., FIG. 9 or one of the TL1A binding domain variants described herein). In exemplary embodiments, the TL1A antigen binding domain includes a vhCDR1 having an amino sequence of SEQ ID NO:2, a vhCDR2 having an amino acid sequence of SEQ ID NO:3, a vhCDR3 having an amino acid sequence of SEQ ID NO:4, a vlCDR1 having an amino acid sequence of SEQ ID NO:6, a vlCDR2 having an amino acid sequence of SEQ ID NO:7 and a vlCDR3 having an amino acid sequence having an amino acid sequence of SEQ ID NO:8. In exemplary embodiments, the TL1A antigen binding domain includes a variable heavy domain having an amino acid sequence of SEQ ID NO:1 and a variable light domain having an amino acid sequence of SEQ ID NO:5. In an exemplary embodiment, the anti-TL1A antibody includes a heavy chain having an amino acid sequence of SEQ ID NO:123, and a light chain having an amino acid sequence of SEQ ID NO:124.

In exemplary embodiments, the anti-TL1a antibody includes a TL1A antigen binding domain that includes a variable heavy domain having an amino acid sequence of SEQ ID NO:1 and a variable light domain having an amino acid sequence of SEQ ID NO:5. In exemplary embodiments, the anti-TL1A antibody further includes a variant Fc domain, wherein the variant Fc domain is a variant dimeric human IgG Fc domain that includes the variants E233P/L234V/L235A/G236del/S267K and M428L/N434S, wherein numbering is according to EU numbering.

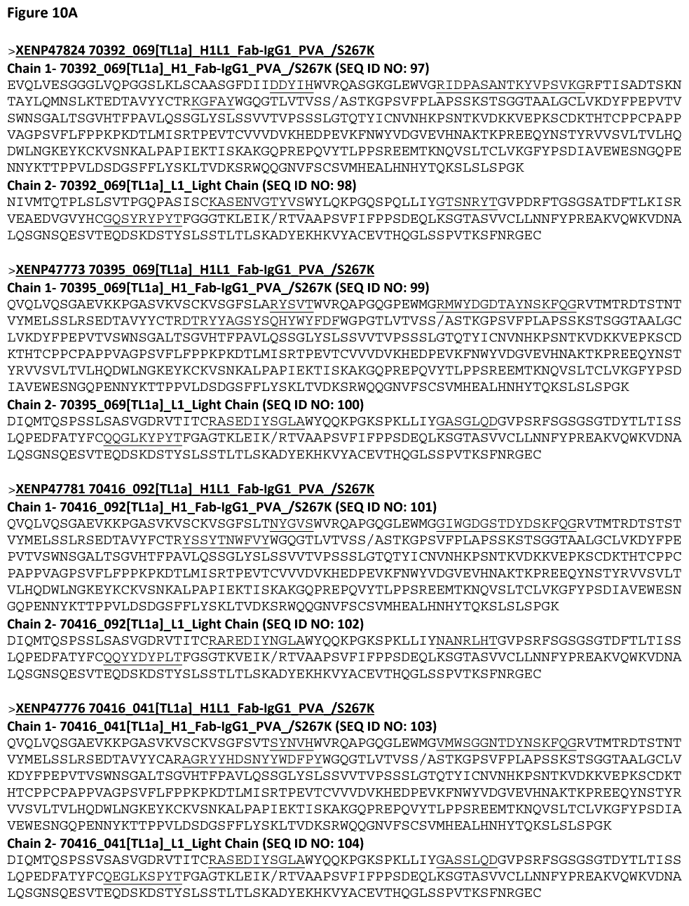

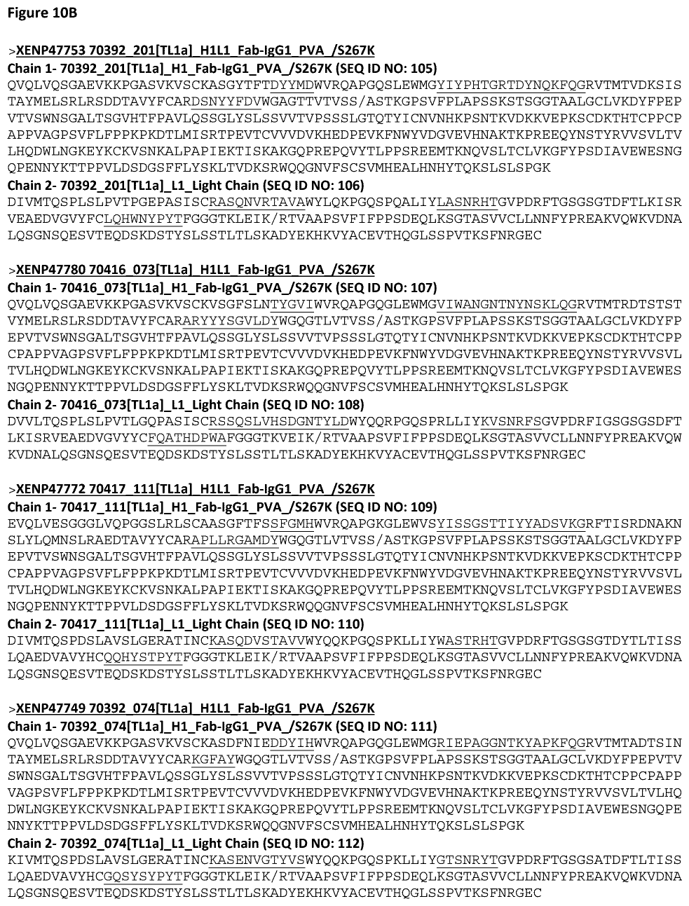

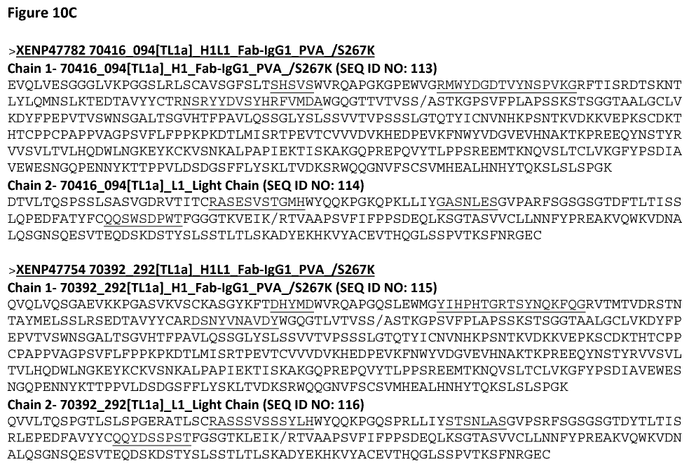

Exemplary monospecific and bivalent subject antibodies are depicted in FIGS. 10-12. In some embodiments, the anti-TL1A antibody comprises a heavy chain and a light chain of an anti-TL1A antibody depicted in FIGS. 10-12. In exemplary embodiments, the anti-TL1A comprises a heavy chain having the amino acid sequence of SEQ ID NO: 123, and a light chain having the amino acid sequence of SEQ ID NO: 124.

### A. Fc Variants for Additional Functionality

In embodiments, the antibodies described herein includes a dimeric Fc domain that includes a first and second monomeric Fc domain. In some embodiments, the dimeric Fc domain is a variant of a wild-type dimeric Fc domain. In some embodiments, the dimeric Fc domain is a variant of a wild-type human IgG (e.g., IgG1, IgG2, and IgG4) Fc domain. There are a number of useful Fc amino acid modification that can be made for a variety of reasons, including, but not limited to, altering binding to one or more FcγR receptors, altered binding to FcRn receptors, etc., as discussed below. Each set of variants can be independently and optionally included or excluded from any particular variant Fc domain described herein. In some embodiments, the variant Fc domain includes one or more FcγR variant described herein. In some embodiments, variant Fc domain includes one or more ablation variant described herein. In exemplary embodiments, the variant Fc domain includes a combination of one or more FcγR variant (e.g., M428L/N434S) and one or more ablation variant (e.g., E233P/L234V/L235A/G236del/S267K or L234A/L235A/P329A). In some embodiments, the variant Fc domain is a variant dimeric human IgG Fc domain that includes the variants M428L/N434S, wherein numbering is according to EU numbering. In some embodiments, the variant Fc domain is a variant dimeric human IgG Fc domain that includes the variants E233P/L234V/L235A/G236del/S267K, wherein numbering is according to EU numbering. In some embodiments, the variant Fc domain is a variant dimeric human IgG Fc domain that includes the variants E233P/L234V/L235A/G236del/S267K and M428L/N434S, wherein numbering is according to EU numbering.

### 1. FcγR Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T, according to EU numbering. Such modification may be included in one or both Fc domains of the subject antibody.

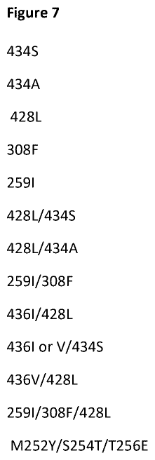

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 259I/308F, 436I/428L, 436I or V/434S, 436V/428L, 428L/434A, M252Y/S254T/T256E, and 259I/308F/428L. Such modification may be included in one or both Fc domains of the subject antibody. In exemplary embodiments, the antibody includes 428L/434S modifications (EU numbering). Additional Fc modifications that increase serum-half of the subject antibody provided herein are depicted in FIG. 7.

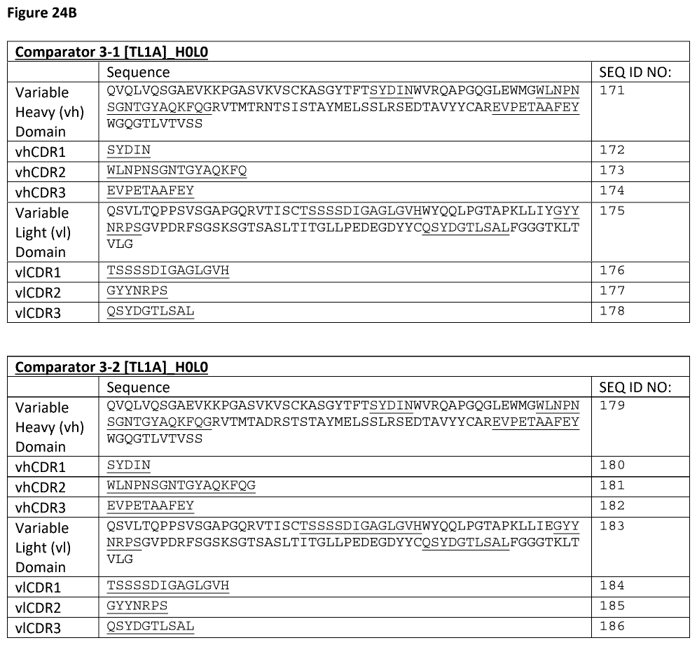

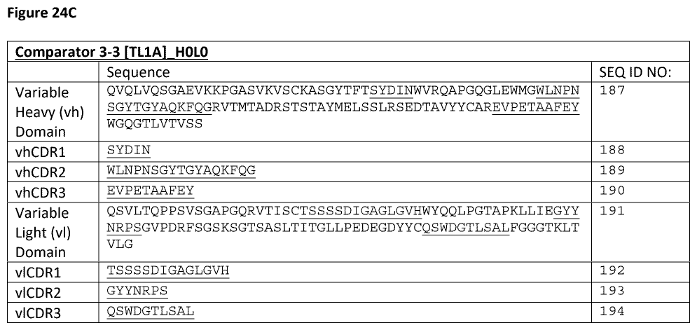

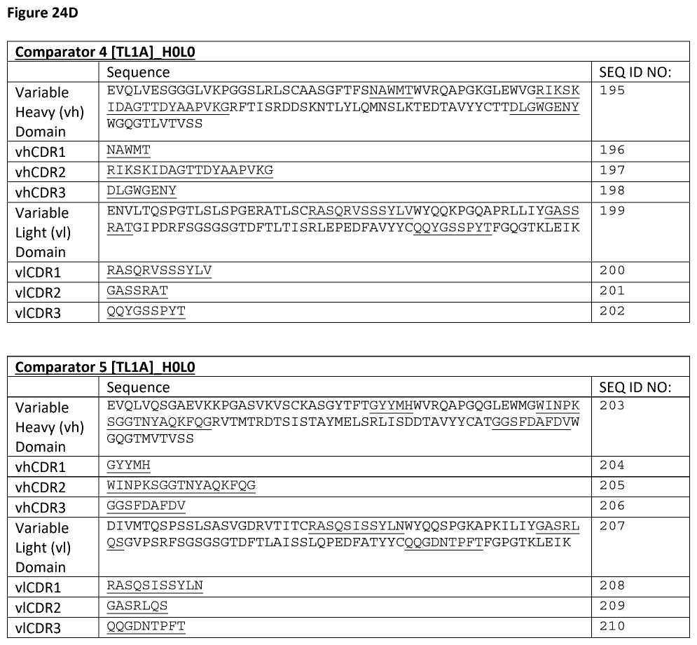

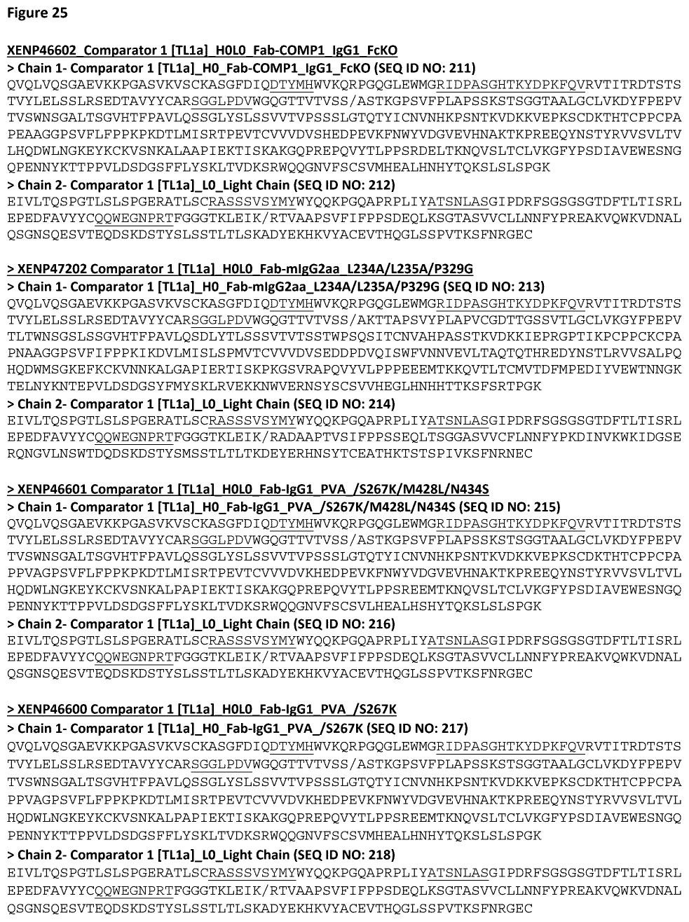

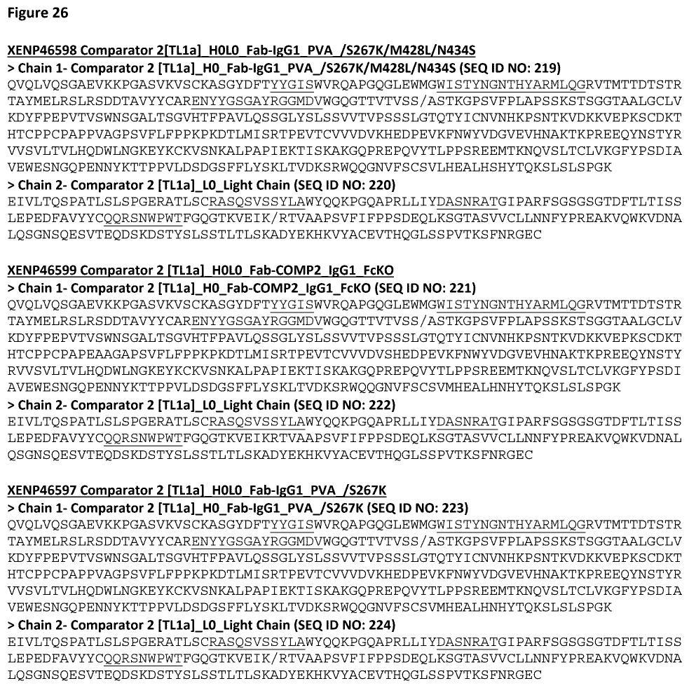

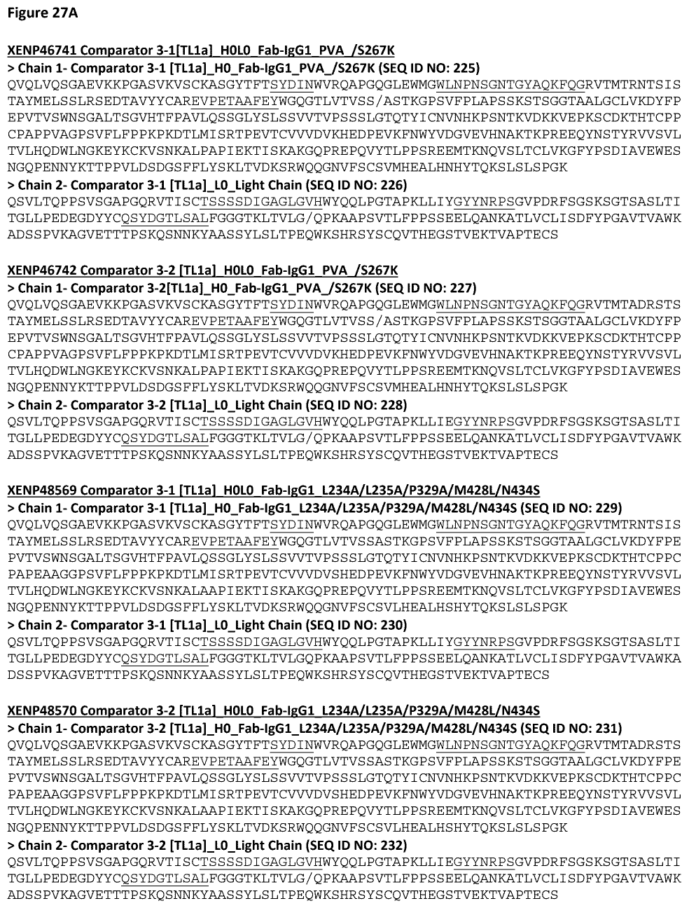

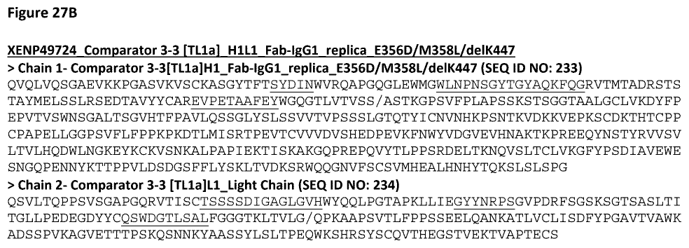

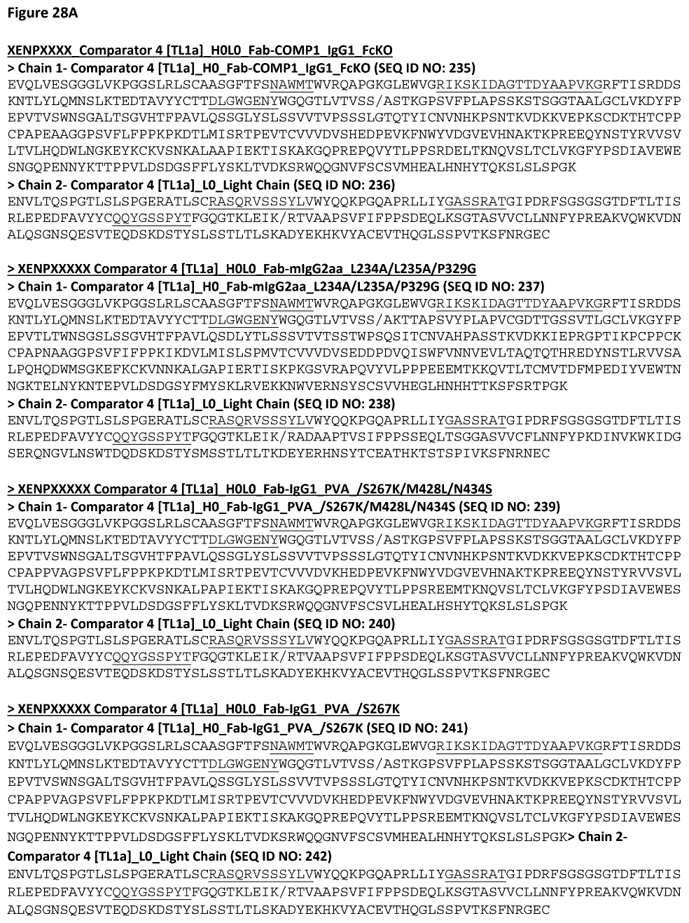

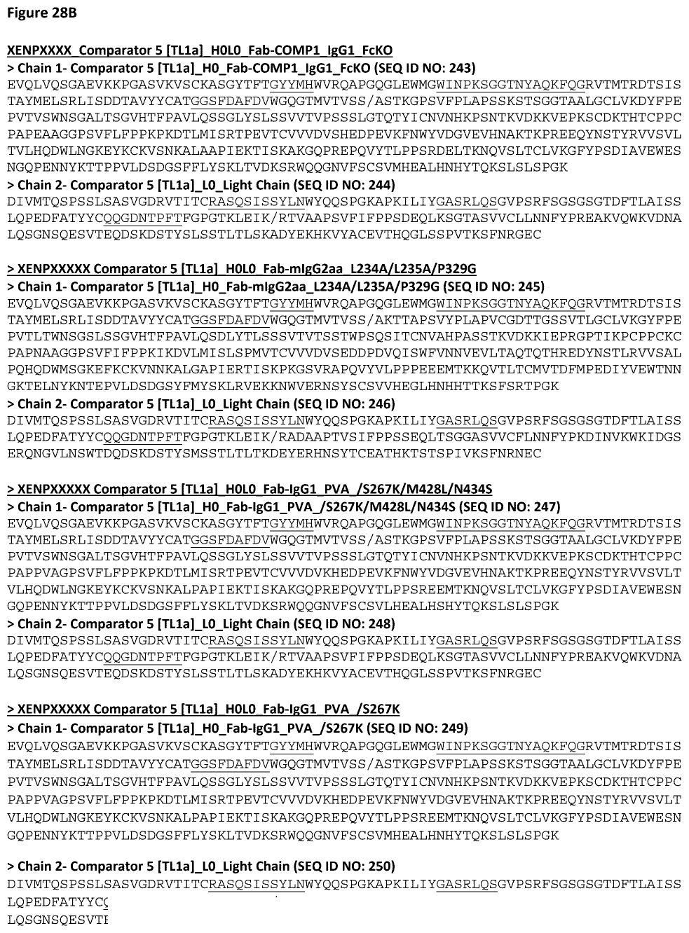

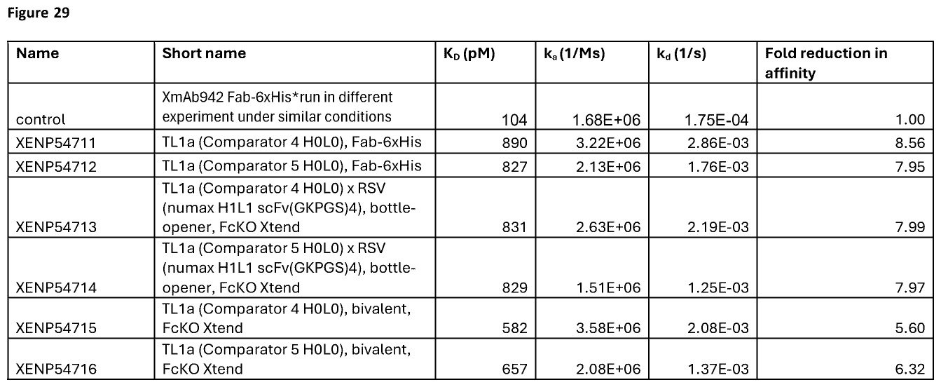

In some embodiments, the amino acid variants 428L/434S Fc variants are included in antibodies that include the comparator VH and VL sequences listed in FIG. 24. Thus, for example, the VH and VL sequences listed in FIG. 24 can be added to human IgG1, IgG2 or IgG4 backbones that include the 428L/434S amino acid variants.

In some embodiments, the amino acid variants M252Y/S254T/T256E Fc variants are included in antibodies that include the comparator VH and VL sequences listed in FIG. 24. Thus, for example, the VH and VL sequences listed in FIG. 24 can be added to human IgG1, IgG2 or IgG4 backbones that include the M252Y/S254T/T256E amino acid variants.

### 2. Ablation Variants

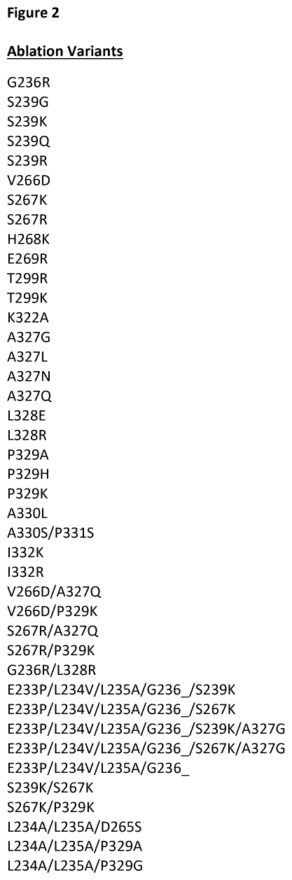

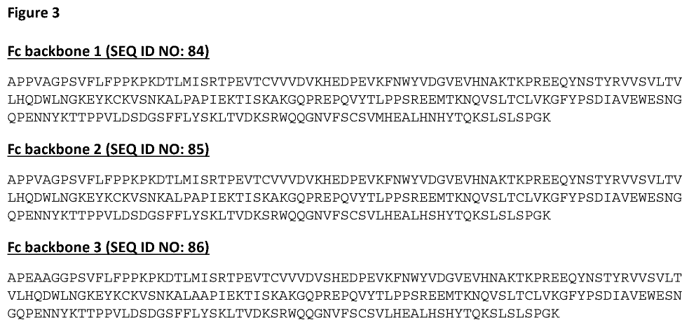

In some embodiments, the antibody provided herein includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprise one or more Fcγ receptor ablation variants. These ablation variants each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G, E233P/L234V/L235A/G236del, and L234A/L235A/P329A (EU numbering). It should be noted that the ablation variants referenced herein ablate FcγR binding but generally not FcRn binding. Additional ablation variants that can be included in the variant Fc domains provided herein are show in FIG. 2.

As is known in the art, the Fc domain of human IgG1 has the highest binding to the Fcγ receptors, and thus ablation variants can be used when the constant domain (or Fc domain) in the backbone of the subject antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### 3. Monospecific, Monoclonal Antibodies

As will be appreciated by those in the art, the novel Fv sequences outlined herein can also be used in both monospecific antibodies (e.g., “traditional monoclonal antibodies”). Accordingly, the present invention provides monospecific antibodies comprising the 6 CDRs and/or the VH and VL sequences from the figures, generally with IgG1, IgG2, IgG3 or IgG4 constant regions, with IgG1, IgG2 and IgG4 (including IgG4 constant regions comprising a S228P amino acid substitution) finding particular use in some embodiments. That is, any sequence herein with a “H_L” designation can be linked to the constant region of a human IgG1 antibody. In some embodiments, the monospecific antibody includes a monomer having, for N- to C-terminus, VH-CH1-hinge-CH2-CH3; and a light chain that includes a VL-CL.

In certain embodiments, the monospecific anti-TL1A antibody includes a TL1A binding domains described herein (e.g., FIG. 9 or one of the TL1A binding domain variants described herein).

Exemplary monospecific and bivalent subject antibodies are depicted in FIGS. 10-12. In some embodiments, the antibody is a bivalent, monospecific antibody that includes a variable heavy domain and variable light domain of any of the subject TL1A binding domains provided herein. In some embodiments, the bivalent, monospecific antibody includes the variable heavy and variable light domain of TL1A binding domain 70416_041[TL1a]_H1L1 (see FIG. 9A). In some embodiments, the antibody includes a dimeric variant Fc domain that includes one or more FcγR variant described herein. In some embodiments, the antibody includes a dimeric variant Fc domain that includes one or more ablation variant described herein. In exemplary embodiments, the variant Fc domain includes a combination of one or more FcγR variant (e.g., M428L/N434S) and one or more ablation variant (e.g., E233P/L234V/L235A/G236del/S267K or L234A/L235A/P329A). In some embodiments, the variant Fc domain is a variant dimeric human IgG Fc domain that includes the variants M428L/N434S, wherein numbering is according to EU numbering. In some embodiments, the variant Fc domain is a variant dimeric human IgG Fc domain that includes the variants E233P/L234V/L235A/G236del/S267K, wherein numbering is according to EU numbering. In some embodiments, the variant Fc domain is a variant dimeric human IgG Fc domain that includes the variants E233P/L234V/L235A/G236del/S267K and M428L/N434S, wherein numbering is according to EU numbering.

### V. Recombinant Methods and Compositions

In another aspect, provided herein are nucleic acid compositions encoding the TL1A antigen binding domains and anti-TL1A antibodies provided herein.

As is known in the art, the nucleic acids encoding the components of the binding domains and antibodies disclosed herein can be incorporated into expression vectors as is known in the art and depends on the host cells used to produce the subject antibodies of the invention. Generally, the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

The antibodies and ABDs provided herein are made by culturing host cells comprising the expression vector(s) as is well known in the art. Once produced, traditional antibody purification steps are done, including an ion exchange chromatography step (e.g., techniques that utilize IEF gels, cIEF, and analytical IEX columns).

### VI. Biological and Biochemical Functionality of the anti-TL1A Antibodies

Generally, the anti-TL1A antibodies described herein are administered to patients with a TL1A-associated disease, disorder, or condition, and efficacy is assessed, in a number of ways as described herein. In some embodiments, the method includes administering to a subject having a TL1A-associated disease a therapeutically effective amount of a subject TL1A antibody as provided herein. In some embodiments, the TL1A associated disease is selected from one of the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis. Methods for assessing ulcerative colitis are disclosed, for example, in Schroeder et al., N Engl J Med 317:1625-9 (1987). Methods for assessing Crohn's Disease and fibrostenotic Crohn's Disease are disclosed for example, in Veauthier and Hornecker, Am Fam Physician 98(11):661-669 (2018) and Solitano et al., J Clin Med. 12(9):3052 (2023). Methods for assessing systemic sclerosis are disclosed, for example, in Lafaytis and Valenzi et al., Nature Reviews Rheumatology 18:527-541 (2022). Methods for assessing interstitial lung disease are disclosed, for example, in Gulati, Primary Care Respiratory Journal 20:120-127 (2011). Methods of assessing primary biliary cirrhosis are disclosed, for example in Bowlus and Gershwin, Autoimmun Rev. 13(0):441-444 (2014). Methods for assessing primary sclerosing cholangitis are disclosed, for example, in Modha and Navaneethan, World J. Hepatol 7(5):799-805 (2015). Methods for assessing rheumatoid arthritis are disclosed, for example, in Wasserman, Am Fam Physician 84(11):1245-1252 (2011). Methods for assessing atopic dermatitis are disclosed, for example, in Fishbein et al., J Allergy Clin Immunol Pract. 8(1):91-101 (2019). Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### VII. Antibody Compositions for In Vivo Administration

Formulations of the antibodies used in accordance with the present invention are prepared for storage by mixing an antibody having the desired degree of purity with optional pharmaceutically acceptable carriers, excipients, or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ PLURONICS™ or polyethylene glycol (PEG).

In one aspect, provided herein is a pharmaceutical composition comprising an TL1A antigen binding domain and a pharmaceutically acceptable carrier. In some embodiments, the TL1A antigen binding domain is any one of the TL1A antigen binding disclosed herein, including, for example those disclosed in FIG. 9. In some embodiments, the TL1A antigen binding domain includes a vhCDR1 having an amino sequence of SEQ ID NO:2, a vhCDR2 having an amino acid sequence of SEQ ID NO:3, a vhCDR3 having an amino acid sequence of SEQ ID NO:4, a vlCDR1 having an amino acid sequence of SEQ ID NO:6, a vlCDR2 having an amino acid sequence of SEQ ID NO:7 and a vlCDR3 having an amino acid sequence having an amino acid sequence of SEQ ID NO:8. In exemplary embodiments, the TL1A antigen binding domain includes a variable heavy domain having an amino acid sequence of SEQ ID NO:1 and a variable light domain having an amino acid sequence of SEQ ID NO:5.

In another aspect, provided herein is a pharmaceutical composition comprising an anti-TL1A antibody and a pharmaceutically acceptable carrier, wherein the anti-TL1A antibody is any one of the anti-TL1A antibodies disclosed herein, including, for example those disclosed in FIGS. 10-12. In some embodiments, the anti-TL1A antibody comprises a TL1A antigen binding domain of any of the TL1A antigen binding domains disclosed in FIG. 9. In exemplary embodiments, the TL1A antigen binding domain includes a vhCDR1 having an amino sequence of SEQ ID NO:2, a vhCDR2 having an amino acid sequence of SEQ ID NO:3, a vhCDR3 having an amino acid sequence of SEQ ID NO:4, a vlCDR1 having an amino acid sequence of SEQ ID NO:6, a vlCDR2 having an amino acid sequence of SEQ ID NO:7 and a vlCDR3 having an amino acid sequence having an amino acid sequence of SEQ ID NO:8. In exemplary embodiments, the TL1A antigen binding domain includes a variable heavy domain having an amino acid sequence of SEQ ID NO:1 and a variable light domain having an amino acid sequence of SEQ ID NO:5. In an exemplary embodiment, the anti-TL1A antibody includes a heavy chain having an amino acid sequence of SEQ ID NO:123, and a light chain having an amino acid sequence of SEQ ID NO:124.

### VIII. Therapeutic Uses

Provided herein are methods of reducing TL1A mediated activity and methods of treating TL1A-associated diseases(e.g., an inflammatory bowel disease) using the subject anti-TL1A antibody disclosed herein. In some embodiments, the subject anti-TL1A antibody disclosed herein inhibits or reduces TL1A-associated activity by inhibiting the binding of TL1A to its receptor, Death Receptor 3 (DR3). TL1A is expressed by antigen present cells (e.g., including dendritic cells, B cells and macrophages), CD4+ and CD8+ T cells and endothelial cells and can be expressed on the cell surface or secreted as a soluble cytokine. In some embodiments, the subject anti-TL1A antibody inhibits or reduces TL1A expressed by one of the following cells: a dendritic cell, a B cell, a macrophage, a CD4+ T cells, a CD8+ T cell, or an endothelial cell. In some embodiments, the binding of TL1A to a DR3 express cell. In certain embodiments, the DR3 expressing cell is one of the following cells: a CD4+ T cell, a CD8+ T cell, an NK cell, an NKT cell or a FOXP3+ regulatory T (Treg) cell.

In one aspect, provided herein are methods of inhibiting or reducing TL1A-mediated cell apoptosis using a subject anti-TL1A antibody as described herein. In some embodiments, the method comprises contacting a TL1A expressing cell with a subject anti-TL1A antibody as described herein, thereby inhibiting or reducing the ability of the TL1A expressing cell to mediate cell apoptosis. In some embodiments, the method inhibits or reduces TL1A-mediated cell apoptosis in a subject in need thereof, wherein the method comprises administrating to the subject an anti-TL1A antibody as described herein. In some embodiments, the subject anti-TL1A antibody reduces apoptosis in a cell or a population of cells in a subject that expresses a death receptor 3 (DR3) receptor. In some embodiments, the subject anti-TL1A antibody reduces apoptosis in a cell or a population of cells in a subject that expresses DR3 by preventing the interaction of TL1A and DR3. Without being bound by any particular theory of operation, it is believed that reduction of TL1A-mediated apoptosis can lead to the treatment of a subject that has a TL1A-associated disease (e.g., an inflammatory bowel disease). In some embodiments, the subject is a human subject. In embodiments, the anti-TL1A antibody includes a TL1A binding domain of any one of the TL1A binding domains depicted herein. In some embodiments, the anti-TL1A antibody includes a TL1A binding domain of FIG. 9. In some embodiments, the anti-TL1A antibody is an anti-TL1A antibody disclosed in FIG. 10, 11 or 12. Any suitable technique known in the art can be used for assessing TL1A-mediated apoptosis. For example, the level of apoptosis of the cells can be assessed by detecting activation of caspases or propidium iodide uptake or other known assays. A TL1a-binding protein that reduces the level of apoptosis compared to the level of apoptosis in the absence of the TL1a-binding protein is considered to inhibit or reduce TL1a activity or neutralize TL1a activity through DR3.

In another aspect, provided herein is an anti-TL1A antibody for use in inhibiting or reducing TL1A-mediated cell apoptosis. In one aspect, provided herein is a use of an anti-TL1A antibody for the manufacture of a medicament for inhibiting or reducing TL1A-mediated cell apoptosis. In embodiments, the anti-TL1A antibody includes a TL1A binding domain of any one of the TL1A binding domains depicted herein. In some embodiments, the anti-TL1A antibody includes a TL1A binding domain of FIG. 9. In some embodiments, the anti-TL1A antibody is an anti-TL1A antibody disclosed in FIG. 10, 11 or 12.

In one aspect, provided herein are methods of inhibiting or reducing TL1A-mediated NFkB signaling in a cell or a population of cells in a subject using a subject anti-TL1A antibody as described herein. In some embodiments, the method comprises contacting a TL1A expressing cell with a subject anti-TL1A antibody as described herein, thereby inhibiting or reducing the ability of the TL1A expressing cell to mediate NFkB signaling. In some embodiments, the method inhibits or reduces TL1A-mediated NFkB signaling in a subject in need thereof, wherein the method comprises administrating to the subject an anti-TL1A antibody as described herein. In some embodiments, the subject anti-TL1A antibody reduces NFkB signaling in a cell or a population of cells in a subject that expresses DR3 receptor. In some embodiments, the subject anti-TL1A antibody reduces NFkB signaling in a cell or a population of cells in a subject that expresses DR3 by preventing the interaction of TL1A and DR3. Without being bound by any particular theory of operation, it is believed that reduction of TL1A-mediated NFkB signaling can lead to the treatment of a subject that has a TL1A-associated disease (e.g., an inflammatory bowel disease). In some embodiments, the subject is a human subject. In embodiments, the anti-TL1A antibody includes a TL1A binding domain of any one of the TL1A binding domains depicted herein. In some embodiments, the anti-TL1A antibody includes a TL1A binding domain of FIG. 9. In some embodiments, the anti-TL1A antibody is an anti-TL1A antibody disclosed in FIG. 10, 11 or 12. Any suitable technique known in the art can be used for assessing TL1A-mediated NFkB signaling, for example, those disclosed in Wu and Bruggeman, Methods Mol. Biol. 1155:1-14 (2014).

In another aspect, provided herein is an anti-TL1A antibody for use in inhibiting or reducing TL1A-mediated NFkB signaling. In one aspect, provided herein is a use of an anti-TL1A antibody for the manufacture of a medicament for inhibiting or reducing TL1A-mediated NFkB signaling. In embodiments, the anti-TL1A antibody includes a TL1A binding domain of any one of the TL1A binding domains depicted herein. In some embodiments, the anti-TL1A antibody includes a TL1A binding domain of FIG. 9. In some embodiments, the anti-TL1A antibody is an anti-TL1A antibody disclosed in FIG. 10, 11 or 12.

In another aspect, provided herein are methods of inhibiting or reducing TL1A-induced cytokine secretion in a cell or a population of cells in a subject using a subject anti-TL1A antibody as described herein. In some embodiments, the method comprises contacting a TL1A expressing cell with a subject anti-TL1A antibody as described herein, thereby inhibiting or reducing the ability of the TL1A expressing cell to induce cytokine secretion. In some embodiments, the method inhibits or reduces TL1A-induced cytokine secretion in a subject in need thereof, wherein the method comprises administrating to the subject an anti-TL1A antibody as described herein. In some embodiments, the subject anti-TL1A antibody (e.g., any of the anti-TL1A antibodies in FIGS. 9-11) reduces TL1A-induced cytokine secretion in a cell or a population of cells in a subject that expresses DR3 receptor. In some embodiments, the subject anti-TL1A antibody reduces cytokine secretion in a cell or a population of cells in a subject that expresses DR3 by preventing the interaction of TL1A and DR3. In some embodiments, the anti-TL1A antibody reduces the secretion of one or more of the following cytokines: interleukin-1β (IL-1β); tumor necrosis factor-α (TNF-α); transforming growth factor-β (TGF-β); granulocyte-macrophage colony stimulating factor (GM-CSF); and interferon-γ (IFN-γ). Without being bound by any particular theory of operation, it is believed that reduction of TL1A-mediated NFkB signaling can lead to the treatment of a subject that has a TL1A-associated disease (e.g., an inflammatory bowel disease). In some embodiments, the subject is a human subject. In embodiments, the anti-TL1A antibody includes a TL1A binding domain of any one of the TL1A binding domains depicted herein. In some embodiments, the anti-TL1A antibody includes a TL1A binding domain of FIG. 9. In some embodiments, the anti-TL1A antibody is an anti-TL1A antibody disclosed in FIG. 10, 11 or 12. Any suitable technique known in the art can be used for assessing TL1A-cytokine secretion, for example, using ELISA.

In another aspect, provided herein is an anti-TL1A antibody for use in inhibiting or reducing TL1A-induced cytokine secretion. In one aspect, provided herein is a use of an anti-TL1A antibody for the manufacture of a medicament for inhibiting or reducing TL1A-induced cytokine secretion. In embodiments, the anti-TL1A antibody includes a TL1A binding domain of any one of the TL1A binding domains depicted herein. In some embodiments, the anti-TL1A antibody reduces the secretion of one or more of the following cytokines: interleukin-1β (IL-1β); tumor necrosis factor-α (TNF-α); transforming growth factor-β (TGF-β); granulocyte-macrophage colony stimulating factor (GM-CSF); and interferon-γ (IFN-γ). In some embodiments, the anti-TL1A antibody includes a TL1A binding domain of FIG. 9. In some embodiments, the anti-TL1A antibody is an anti-TL1A antibody disclosed in FIG. 10, 11 or 12.

In another aspect, provided herein are methods for the treatment of a TL1A-associated disease, disorder, or condition. In some embodiments, the method includes administering to a subject having a TL1A-associated disease a therapeutically effective amount of a subject TL1A antibody as provided herein. In some embodiments, the TL1A-associated disease is an inflammatory bowel disease. In some embodiments, the TL1A associated disease is selected from one of the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis. Any suitable method known in the art may be used to assess the TL1A-associated disease, including any of the methods disclosed herein. In certain embodiments, the subject is a human patient. In embodiments, the anti-TL1A antibody includes a TL1A binding domain of any one of the TL1A binding domains depicted herein. In some embodiments, the anti-TL1A antibody includes a TL1A binding domain of FIG. 9. In some embodiments, the anti-TL1A antibody is an anti-TL1A antibody disclosed in FIG. 10, 11 or 12.

In another aspect, provided herein is an anti-TL1A antibody for use in the treatment of a TL1A-associated disease. In one aspect, provided herein is a use of an anti-TL1A antibody for the manufacture of a medicament for i treatment of a TL1A-associated disease. In some embodiments, the TL1A associated disease is selected from one of the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis. In embodiments, the anti-TL1A antibody includes a TL1A binding domain of any one of the TL1A binding domains depicted herein. In some embodiments, the anti-TL1A antibody includes a TL1A binding domain of FIG. 9. In some embodiments, the anti-TL1A antibody is an anti-TL1A antibody disclosed in FIG. 10, 11 or 12. In some embodiments, the anti-TL1A antibody reduces the secretion of one or more of the following cytokines: interleukin-1β (IL-1β); tumor necrosis factor-α (TNF-α); transforming growth factor-β (TGF-P); granulocyte-macrophage colony stimulating factor (GM-CSF); and interferon-γ (IFN-γ). In some embodiments, the anti-TL1A antibody includes a TL1A binding domain of FIG. 9. In some embodiments, the anti-TL1A antibody is an anti-TL1A antibody disclosed in FIG. 10, 11 or 12.

The antibodies provided herein administered to a subject, in accord with known methods, such as intravenous administration as a bolus, intravenous push or by intravenous infusion over a period of time. In some embodiments, the anti-TL1A antibody provided herein is administered by intravenous infusion. In some embodiments, the anti-TL1A antibody provided herein is administered subcutaneously.

In the methods of the invention, therapy is used to provide a positive therapeutic response with respect to a disease, disorder or condition described herein. Positive therapeutic responses in any given disease, disorder or condition can be determined by standardized response criteria specific to that disease or condition.

In addition to these positive therapeutic responses, the subject undergoing therapy may experience the beneficial effect of an improvement in the symptoms associated with the disease.

Treatment according to the present invention includes a “therapeutically effective amount” of the anti-TL1A antibody used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The efficient dosages and the dosage regimens for the anti-TL1A antibodies described herein depend on the disease or condition to be treated and may be determined by the persons skilled in the art.

In one aspect, provided herein is method of administering an anti-TL1A antibody or pharmaceutical composition that includes the anti-TL1A antibody to a subject (e.g., a human patient), wherein the anti-TL1A antibody is administered to the subject in a first dosing regimen and a second dosing regimen, and wherein the anti-TL1A antibody is a subject anti-TL1A antibody provided herein. In some embodiments, the first dosing regimen comprises administering the anti-TL1A antibody once every four weeks. In some embodiments, the second dosing regimen is administered once every eight weeks. In some embodiments, the first dosing regimen and second dosing regimen are administered subcutaneously or intravenously. In some embodiments, the first dosing regimen is administered intravenously. In some embodiments, the first dosing regimen is administered subcutaneously. In some embodiments, the second dosing regimen is administered intravenously. In some embodiments, the second dosing regimen is administered subcutaneously. In some embodiments, the first dosing regimen is administered intravenously, and the second dosing regimen is administered subcutaneously. In some embodiments, the anti-TL1A antibody comprises a TL1A antigen binding domain of any of the TL1A antigen binding domains disclosed in FIG. 9. In exemplary embodiments, the TL1A antigen binding domain includes a vhCDR1 having an amino sequence of SEQ ID NO:2, a vhCDR2 having an amino acid sequence of SEQ ID NO:3, a vhCDR3 having an amino acid sequence of SEQ ID NO:4, a vlCDR1 having an amino acid sequence of SEQ ID NO:6, a vlCDR2 having an amino acid sequence of SEQ ID NO:7 and a vlCDR3 having an amino acid sequence having an amino acid sequence of SEQ ID NO:8. In exemplary embodiments, the TL1A antigen binding domain includes a variable heavy domain having an amino acid sequence of SEQ ID NO:1 and a variable light domain having an amino acid sequence of SEQ ID NO:5. In an exemplary embodiment, the anti-TL1A antibody includes a heavy chain having an amino acid sequence of SEQ ID NO:123, and a light chain having an amino acid sequence of SEQ ID NO:124.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are known in the art and outlined in US Publications 2015/0307629, 2014/0288275 and WO2014/145806, all of which are expressly incorporated by reference in their entirety and particularly for the techniques outlined therein.

### Example 1: Generation & Engineering of TL1A Antibodies

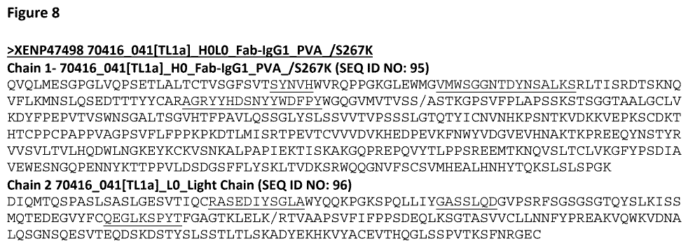

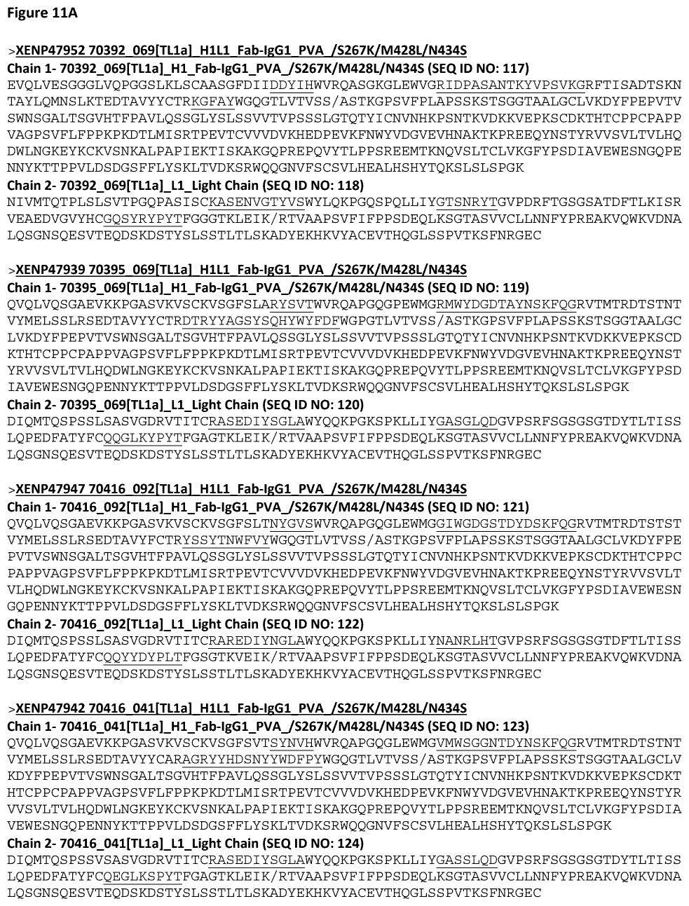

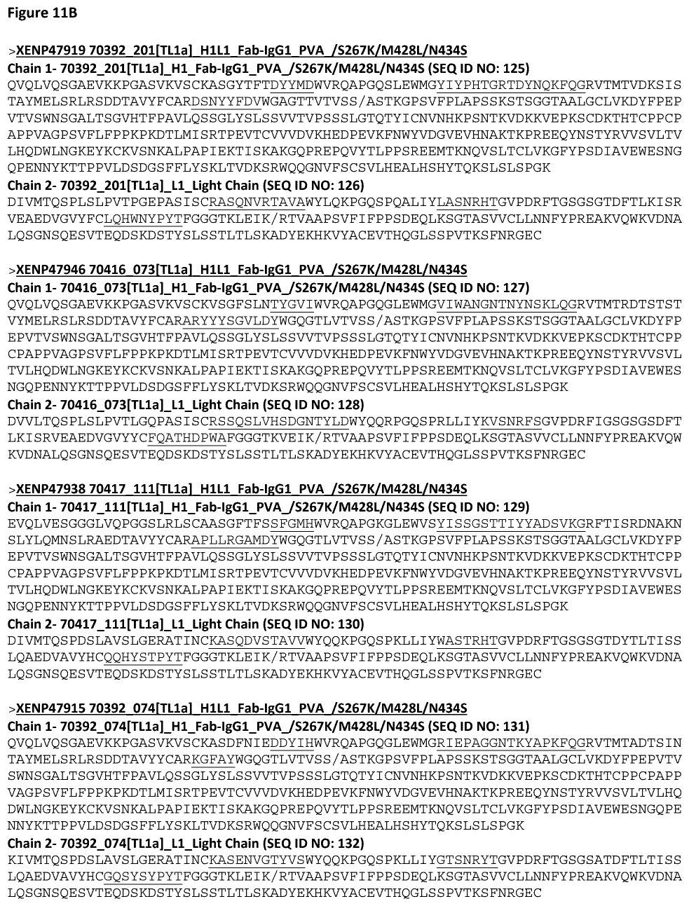

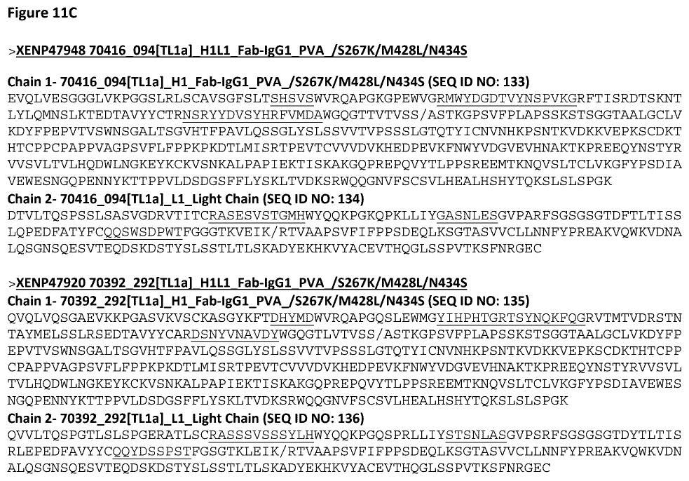

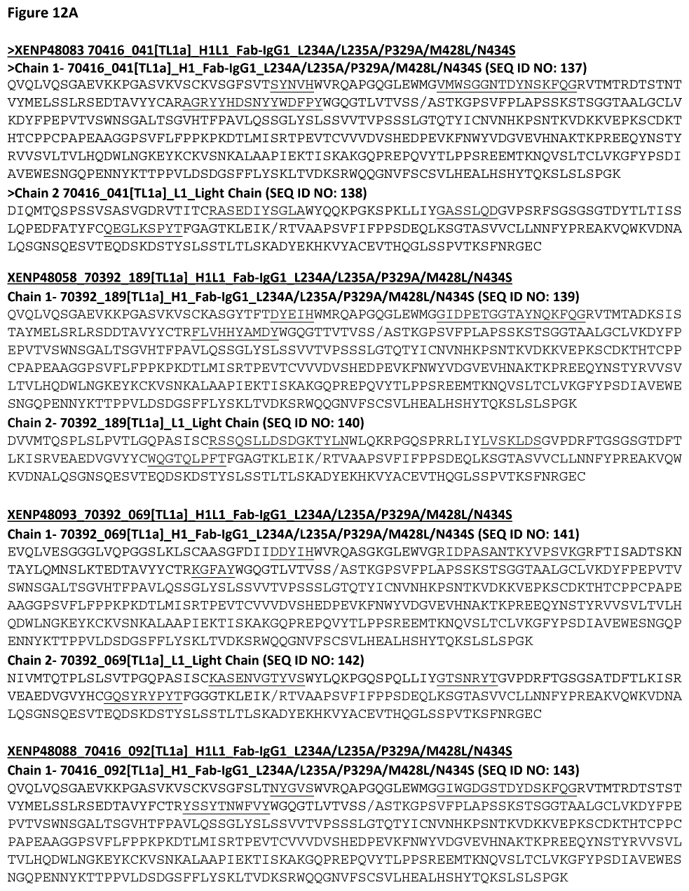

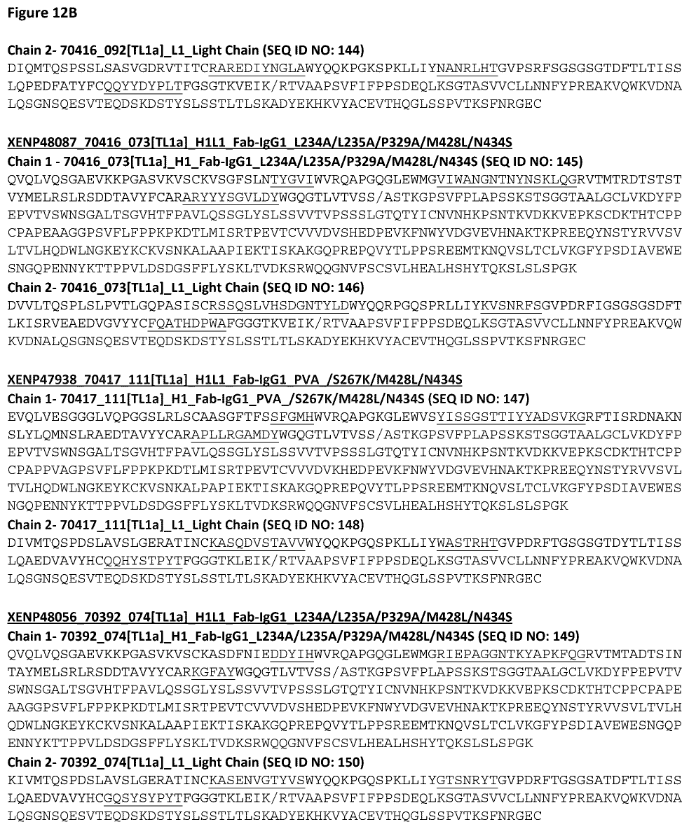

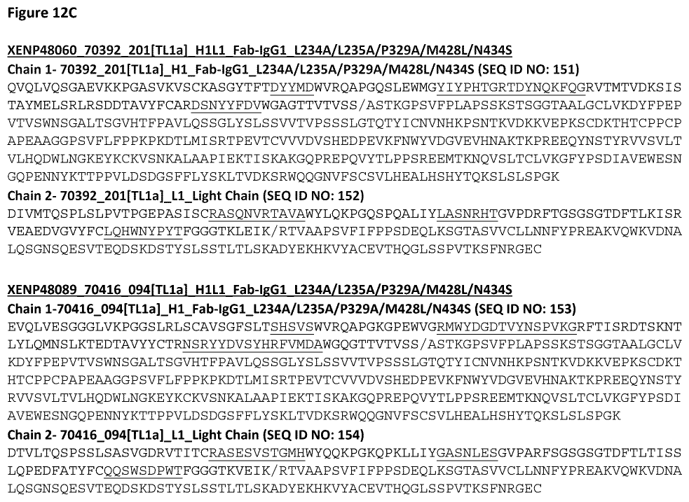

In an initial campaign, numerous novel anti-TL1A mouse and rat antibodies were generated utilizing a single plasma B-cell antibody discovery platform from Single Cell Technologies. The sequence of an exemplary rat antibody XENP47498, having a TL1A binding domain designated 70416_041[TL1A]_H0L0, is depicted in FIG. 8. Antibodies were then humanized using string content optimization (see, e.g., U.S. Pat. No. 7,657,380) and exemplary humanized TL1A binding domains along with their CDRs are depicted in FIG. 9. The Fc region of the antibody was engineered to include variants to ablate effector function (FcKO variants) such as those depicted in FIG. 2 Full length amino acid sequences for TL1A antibodies utilizing the PVA_/S267K FcKO variant are depicted in FIG. 10. The Fc region of the antibody was further engineered to include variants that extend the half-life of the antibody by increased FcRn binding, such as those depicted in FIG. 7. TL1A antibodies with the M428L/N434S variants that extend the half-life of the antibodies, in addition to the PVA_/S267K variant, such as XENP47942 (also referred to as XmAb942), are depicted in FIG. 11. Full length amino acid sequences for a TL1A antibodies alternatively utilizing the L234A/L235A/P329A FcKO variant as well as the M428L/N434S half-life extension variant is depicted in FIG. 12.

### Example 2: Characterization of TL1A Antibodies

### 2a: TL1A Binding Affinity

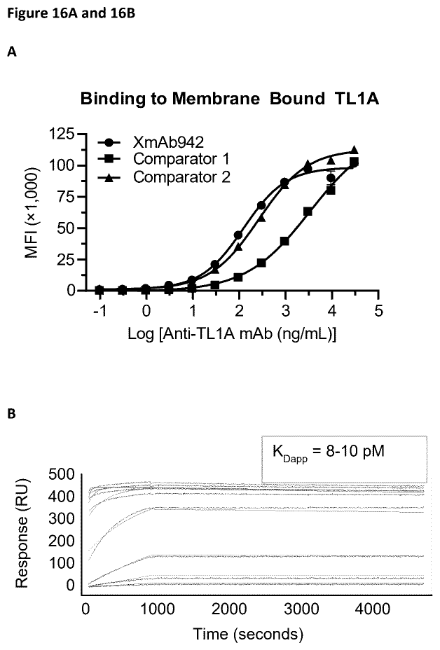

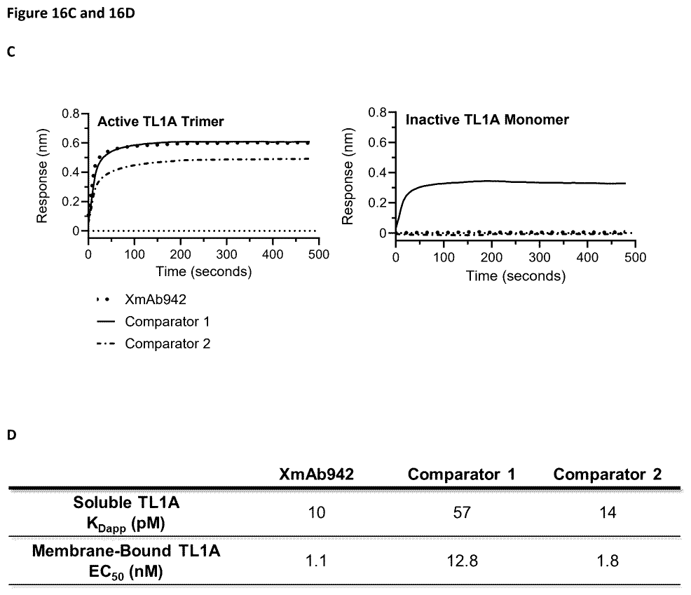

Anti-TL1A mAbs were first screened for binding affinity to membrane-bound TL1A. CHO cells were transiently transfected to express TL1A, then were incubated with anti-TL1A mAbs for 1 hour at 4° C. and analyzed by flow cytometry. Hundreds of anti-TL1A mAbs were screened for binding, in order to determine which were the best binders to investigate further. One exemplary test article, XmAb942 had an EC50 value of 1.1 nM, while Comparator 1 had an EC50 of 12.8 nM, and Comparator 2 had an EC50 of 1.8 nM respectively. The binding data is depicted in FIG. 16A.

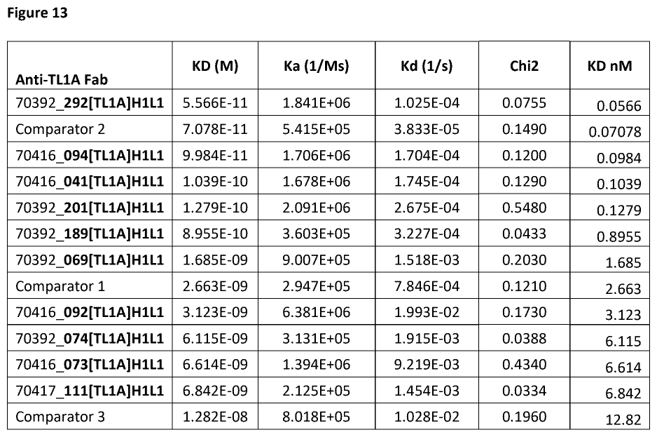

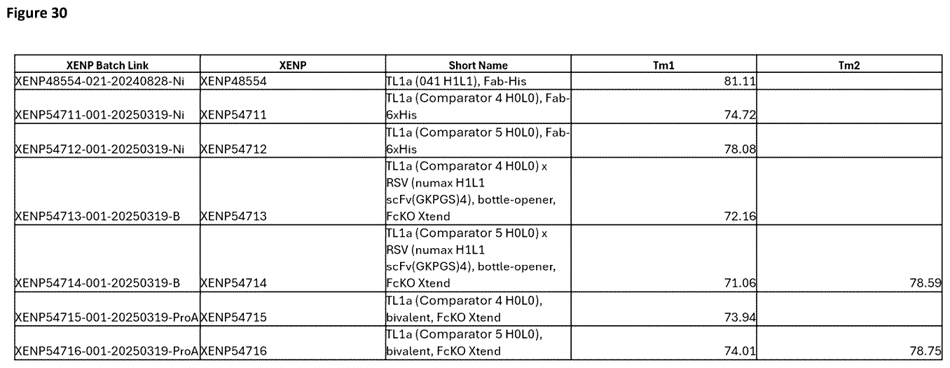

The monovalent binding affinity of anti-TL1A mAbs were assessed with Biacore. Using a Biotin CAPture kit, human scTL1A was captured at 30 nM for 40 seconds at 37° C. Serial dilutions of anti-TL1A Fab were prepared and flowed over scTL1A antigen with a 10 minute association time and 1 hour dissociation time. The results are depicted in FIG. 13. Of note, test articles having TL1A binding domains such as 70416_041[TL1A]H1L1, 70392_292[TL1A]H1L1 and 70416_094[TL1A]H1L1 all displayed significantly tighter affinities than Comparator 1 and Comparator 3. The 70416_041[TL1A]H1L1 binding domain is used in XmAb942. Similar studies further show that the 70416_041[TL1A]H1L1 exhibited significantly tighter affinity than Comparator 4 and Comparator 5 anti-TL1A antibodies. See FIG. 30.

In addition to measuring binding to scTL1A, binding to increasing concentrations of soluble recombinant human TL1A trimer (up to 100 nM) was measured at 37C via surface plasmon resonance. Xmab942 exhibited a K_{Dapp} of 8-10 pM., as depicted in FIG. 16B. Comparator 1 had a K_{Dapp} of 57 pM, and Comparator 2 had 14 pM. This data is summarized in FIG. 16D. Furthermore, the binding of anti-TL1A Fabs (at a concentration of 200 nM) to immobilized active TL1A trimer or inactive TL1A monomer at 25° C. was assessed using biolayer interferometry, and the results depicted in FIG. 16C show that the binding of XmAb942 is specific to the active trimeric form of TL1A. In addition, while XmAb942 binds trimeric TL1A with high affinity, it retains specificity and does not bind to the related TNF ligand superfamily members LIGHT, TRAIL, or FasL.

### 2B: TL1A Antibody XmAb942 is Highly Thermostable

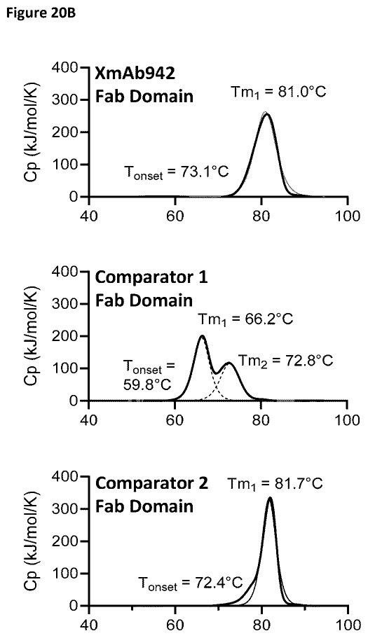

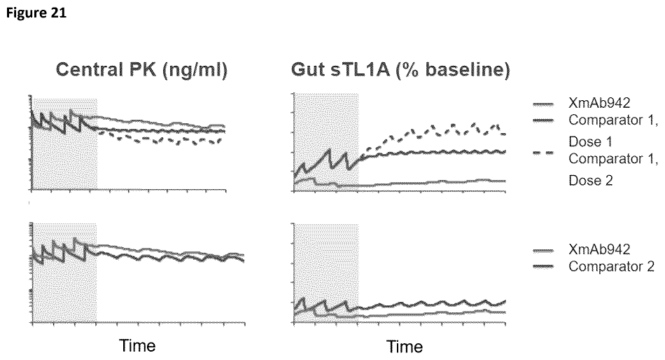

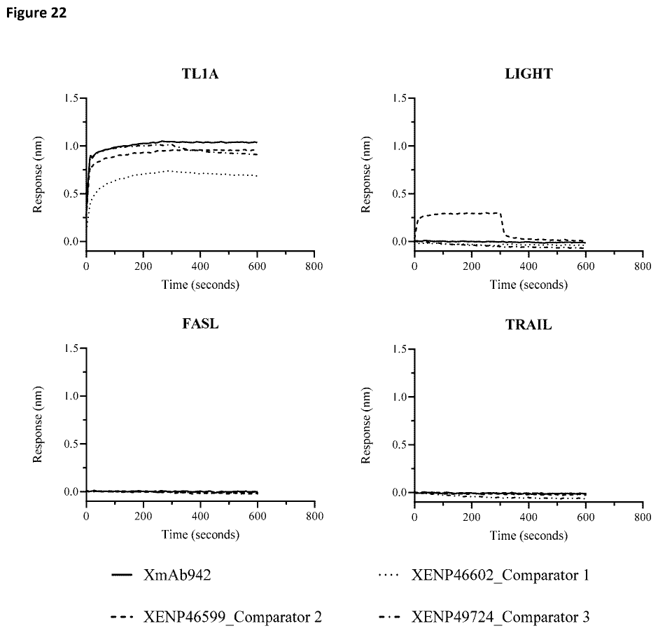

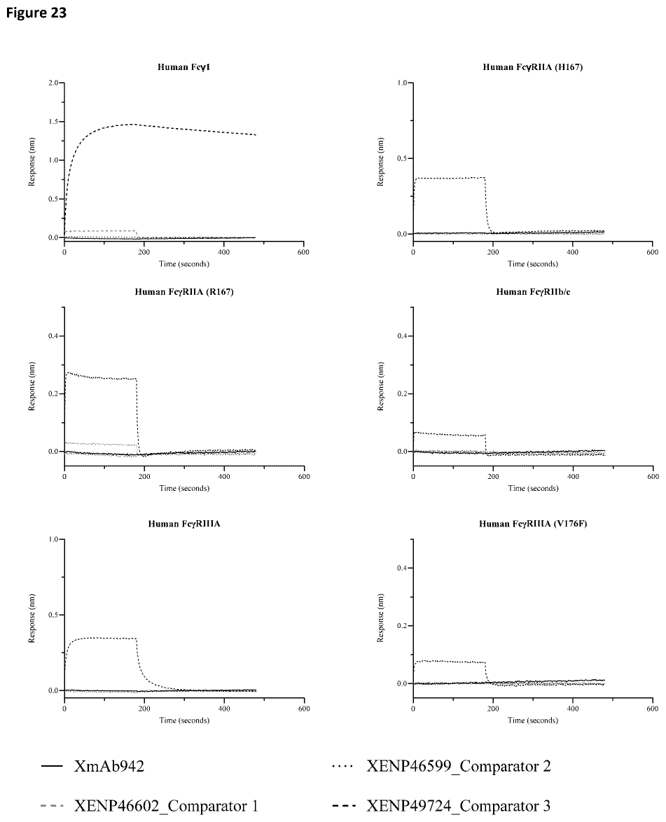

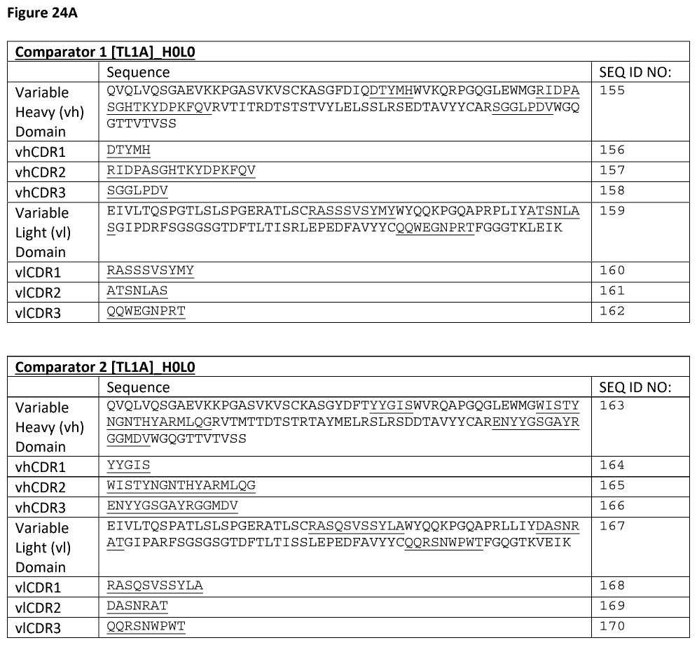

Differential scanning calorimetry was used in order to investigate the thermostability of anti-TL1A mAbs. At a concentration of 1 mg/ml, XmAb942 was evaluated from 25° C. to 95° C. at a ramp rate of 60° C./minute. A Tₒₙₛₑₜ value of 66.0° C. and TM values of 77.2° C. and 75.9° C. were calculated for XmAb942 (FIG. 20A). This data indicates that XmAb942 has an acceptable thermostability profile.

At a concentration of 1 mg/ml, XmAb942 and two comparator antibodies were evaluated from 25° C. to 95° C. at a ramp rate of 1° C./minute. The results, depicted in FIG. 20B, illustrate that XmAb942 has a significantly higher TM₁ of 81° C. relative to Comparator 1, which has a TM₁ of 66.2° C. and a TM₂ of 72.8° C. Comparator 2 has a very similar TM₁ of 81.7° C. relative to XmAb942.

The results depicted in FIG. 20B illustrate that XmAb942 has a significantly higher TM₁ of 81° C. relative to Comparator 1, which has a TM₁ of 66.2° C. and a TM₂ of 72.8° C. Comparator 2 has a very similar TM₁ of 81.7° C. relative to XmAb942.

Further studies show that an anti-TL1A antibody with the 041 [TL1A]H1L1 binding domain of XmAb942 also exhibited a higher TM₁ relative to two additional anti-TL1A antibodies (Comparators 4 and 5). See FIG. 30.

### 2C: TL1A Antibodies Inhibit TL1A Induced TF-1 Apoptosis

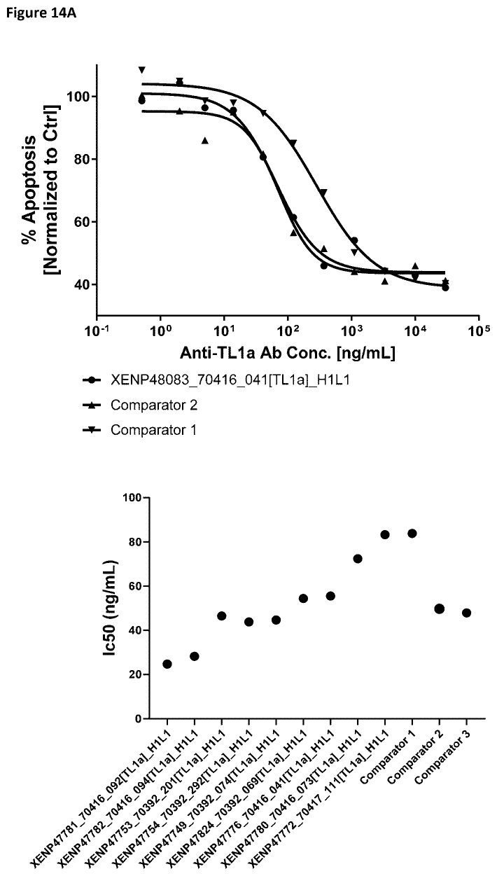

In order to investigate the functional activity of anti-TL1A mAbs described herein, an experiment was conducted in which cycloheximide (1 ug/mL) treated 10K TF-1 cells were seeded in a 384 well plate followed by the addition of variable concentrations of anti-TL1AAb. Subsequently, 0.1 ug/mL recombinant TL1A was added for a 6 hour incubation. Next, Caspase-Glo 3/7 reagent was added for 30 min and luminescence were measured. The results, depicted in FIG. 14, indicate that the TL1A mAbs disclosed herein inhibit caspase activity with competitive IC50 values relative to Comparator clones. A similar experiment was repeated using XENP47942, which is identical to XENP48083 except for that it additionally contains the Xtend variant, and showed the same results as depicted in FIG. 14A.

### 2D: TL1A Antibodies Inhibit TL1A-Induced NFkB Signaling

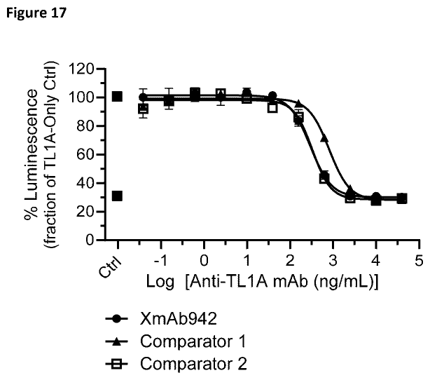

A Jurkat reporter cell line was incubated with anti-TL1A mAbs and 0.4 pg/mL of TL1A, then luciferase activity (reflecting the induction of NFκB) was measured after 5 hours. The results are depicted in FIG. 17. Data are means±standard error of the mean (SEM).

### 2E: TL1A Antibodies Inhibit TL1A-Induced IFNγ Release

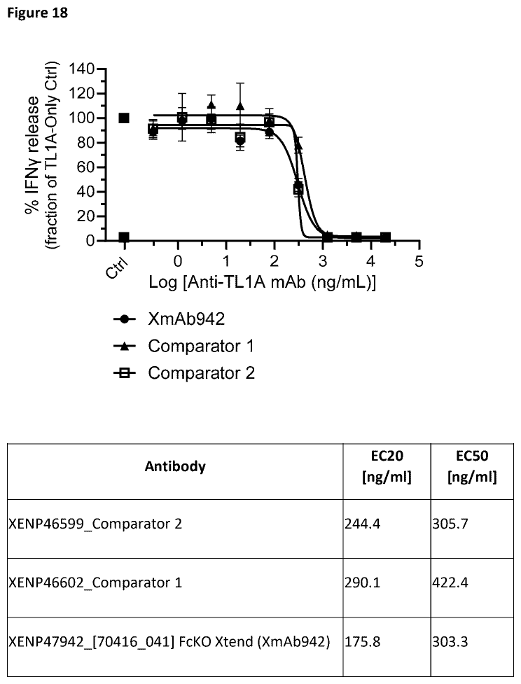

IL12- and IL18-stimulated CD4+ T cells from 8 PBMC donors were treated with anti-TL1A mAbs and 0.2 μg/mL of TL1A, then IFNγ release was measured after 24 hours. The results are depicted in FIG. 18. Data are means±SEM.

### Example 3: Anti-TL1A Half-Life In Vivo

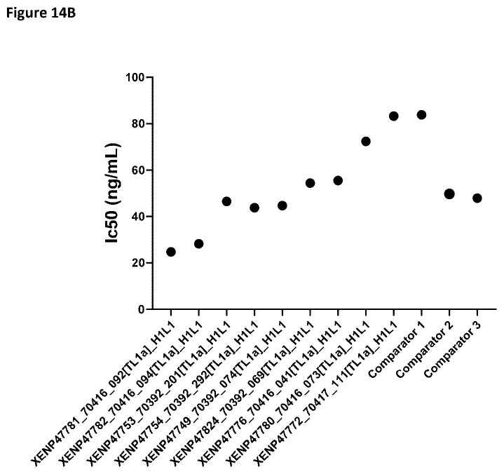

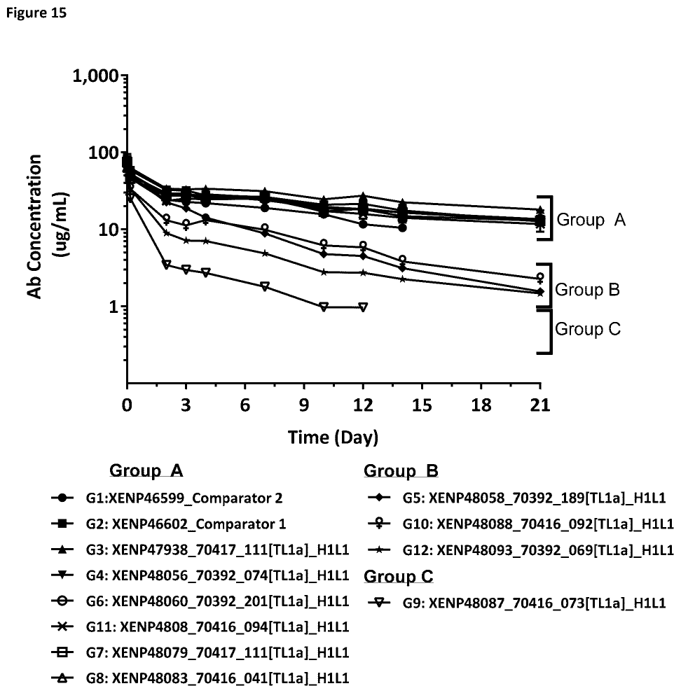

To investigate the pharmacokinetic profile of the anti-TL1A mAbs, a single dose study was conducted in humanized FcRn (huFcRn) mice. All mice were randomly enrolled into study groups by body weight, and each group consisted of 5 mice. After grouping, each mouse was dosed with 5 mg/kg of anti-TL1A mAb intravenously. Retro-orbital bleeds of ˜20-50 ul were collected at Day-1 (pre-dose, pooled from blank control group), 10 min, 4h, Day 2 (48 h), Day 3 (72 h), Day 4 (96h), Day 7 (168 h), Day 10 (240 h), Day 12 (288 h), Day 14 (336 h), and Day 21 (504 h). Blood was collected in anti-coagulant centrifuge tube (EDTA-K2), and then centrifuged at 8000 rpm, 5 minutes, 4° C. to plasma collection for PK ELISA. As depicted in FIG. 15, anti-TL1A mAbs in Group A demonstrated significantly longer half-lives than those in groups B or C. XENP48083 (an XmAb942 analog) in particular was calculated to have a half-life of ˜−14 days. Additionally, the half-life in non-human primate studies supports Q8W-Q12W dosing in humans.

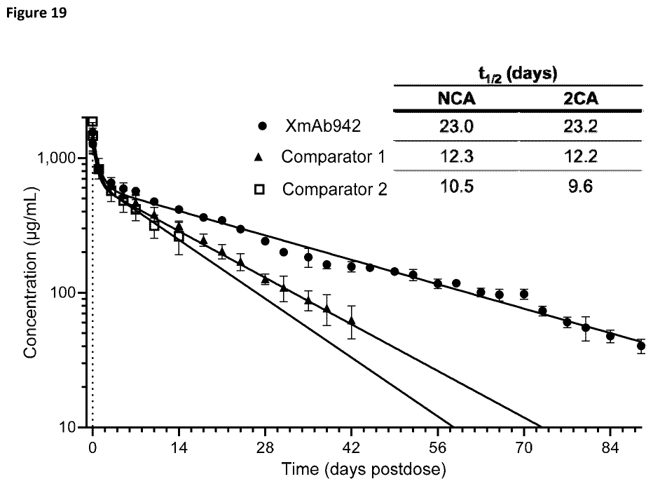

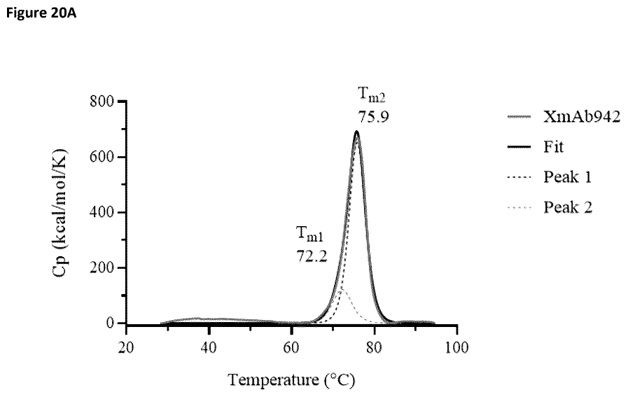

Cynomolgus monkeys received a single intravenous (IV) injection of the indicated anti-TL1A mAbs. Concentration vs time data were analyzed by noncompartmental analysis (NCA) and two-compartmental analysis (2CA). Data depicted in FIG. 19 are means±SEM; n=3 monkeys/group. The vertical dashed line denotes the day of dosing. Allometric scaling adjusted for half-life engineered antibodies predicts that XmAb942 will have a half-life of >70 days in humans, potentially enabling dosing intervals every 8 weeks or longer. XmAb942 was well-tolerated at all doses in a GLP study in cynomolgus monkeys.

### Example 4: Assessment of XmAb942 Dosing Schedule

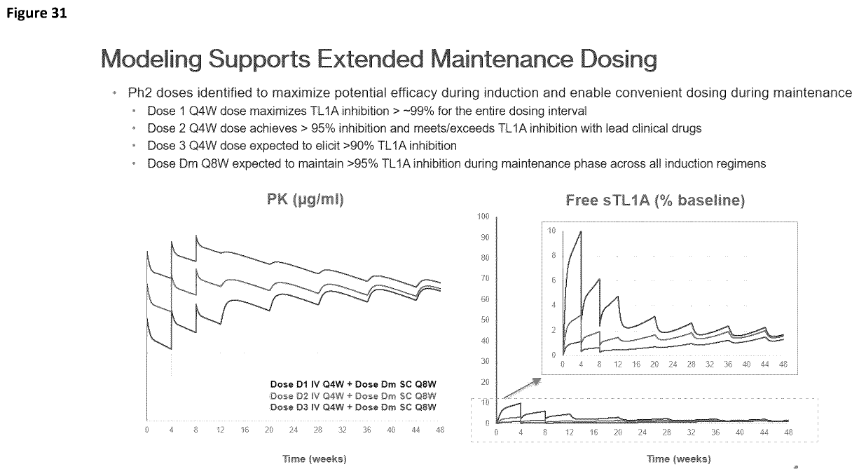

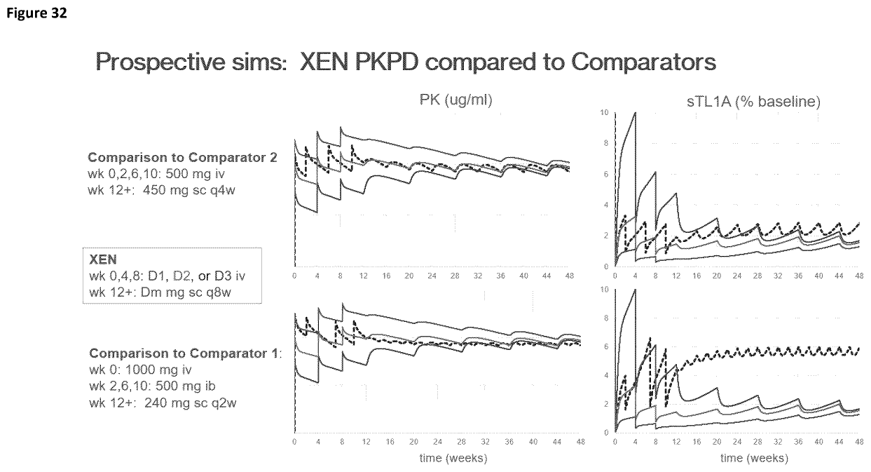

Quantitative Systems Pharmacology (QSP) modeling was used to determine pharmacokinetics (PK) and pharmacodynamics (PD) profiles for three XmAb942 dosing schedules, wherein induction doses are administered every four weeks (qw4, iv) at weeks 0, 4, and 8 at three different dosages (D1, D2, or D3), followed by maintenance doses once every eight weeks (qw8, sc) at dose Dm (FIG. 31). PK and PD profiles were also determined for two comparator anti-TL1A antibodies (“Comparator 1” and “Comparator 2”) at the dosing schedules as shown in FIG. 32. The QSP modeling was based on human PK and PD data for the comparators and non-clinical data for XmAb942.

Based on these studies, it was determined that XmAb942 induction dose D1 q4w dose maximizes TL1A inhibition >˜99% for the entire dosing interval, and XmAb942 induction dose D2 q4w dose achieves >95% inhibition and meets/exceeds TL1A inhibition with Comparator 1 and Comparator 2. Further, XmAb942 maintenance dose Dm q8w doses is expected to maintain >95% TL1A inhibition during maintenance phase across all induction regimens.

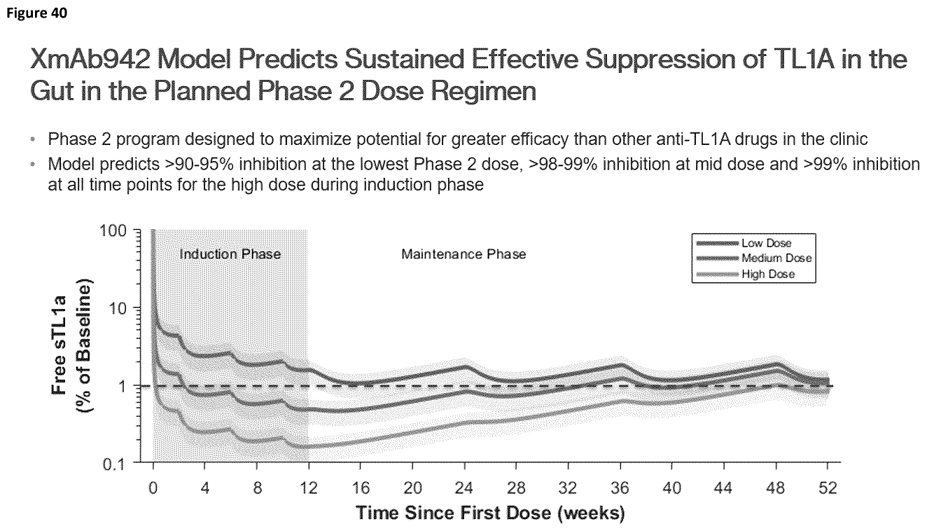

Additional modeling predicted that sustained effective suppression of TL1A in the gut in a planned phase 2 dose regimen (see FIG. 40).

### Example 5: Assessment of XmAb942 Pharmacokinetics and Pharmacodynamics

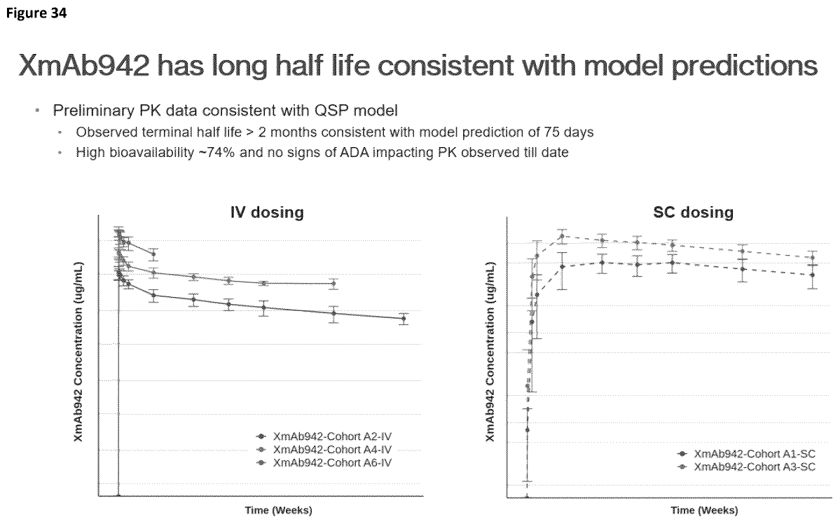

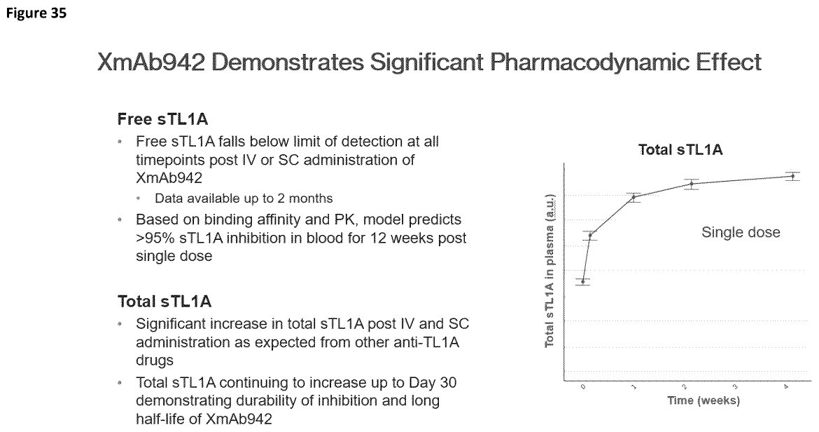

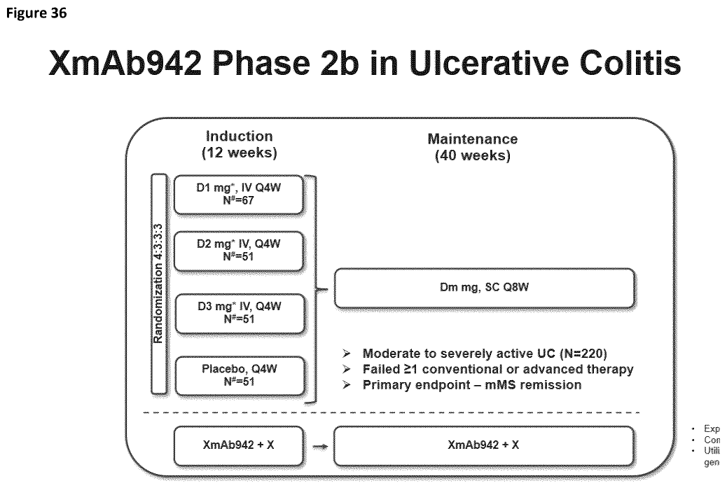

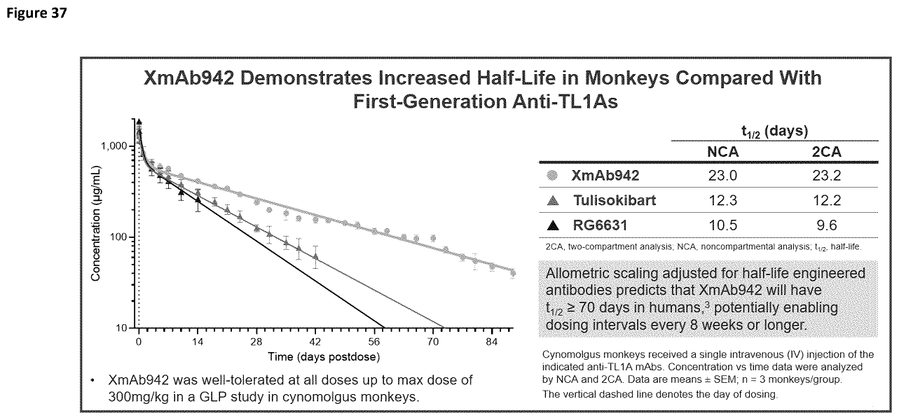

PK and PD properties of XmAb942 were further assessed in humans and the results are summarized in FIGS. 34 and 35. Preliminary PK data for XmAb942 was consistent with the QSP model predictions of Example 4 (FIG. 34). In particular, XmAb942 terminal half-life was observed to be greater than 2 months, consistent with model prediction of 75 days. XmAb942 also exhibited high bioavailability (˜74%) and no signs of anti-drug antibodies (ADA) impacting PK were observed in the study. Longer duration data will be obtained to estimate terminal phase parameters. Additional PK studies demonstrated increased half-life in monkeys compared with first-generation anti-TL1As (FIG. 37).

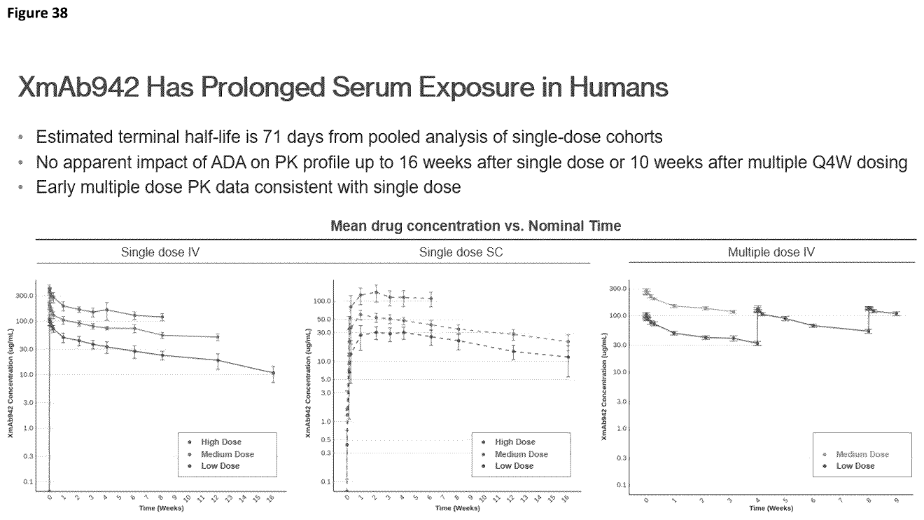

Additional results from a human PK study with three different doses of XmAb942 administered as a single dose IV or SC or multiple dose IV is shown in FIG. 38. Based on the data of the single-dose cohorts, it was estimated that the terminal half-life for XmAb942 is 71 days. There was no apparent impact of ADA on PK profile up to 16 weeks after single dose or 10 weeks after multiple Q4W dosing. Early multiple dose PK data was consistent with single dose.

XmAb942 also exhibited significant pharmacodynamic effects in humans (FIG. 35). In particular, free soluble TL1A (sTL1A) levels fell below limit of detection at all timepoints post IV or SC administration of XmAb942. Based on the binding affinity and PK results for XmAb942, modeling predicted >95% sTL1A inhibition in blood for 12 weeks post single dose. Total sTL1A levels significantly increased post IV and SC administration, as expected from other anti-TL1A drugs. Moreover, such sTL1A levels increased up to Day 30, demonstrating the durability of TL1A inhibition and long half-life of XmAb942.

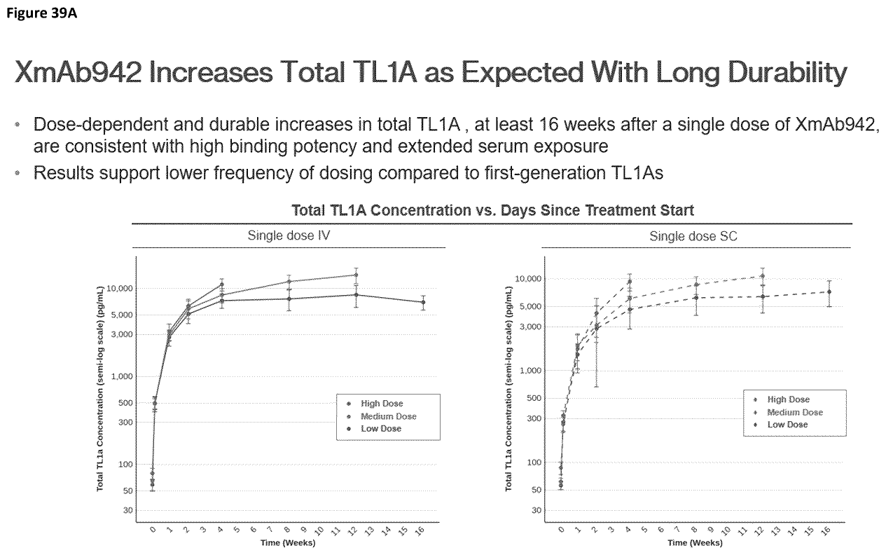

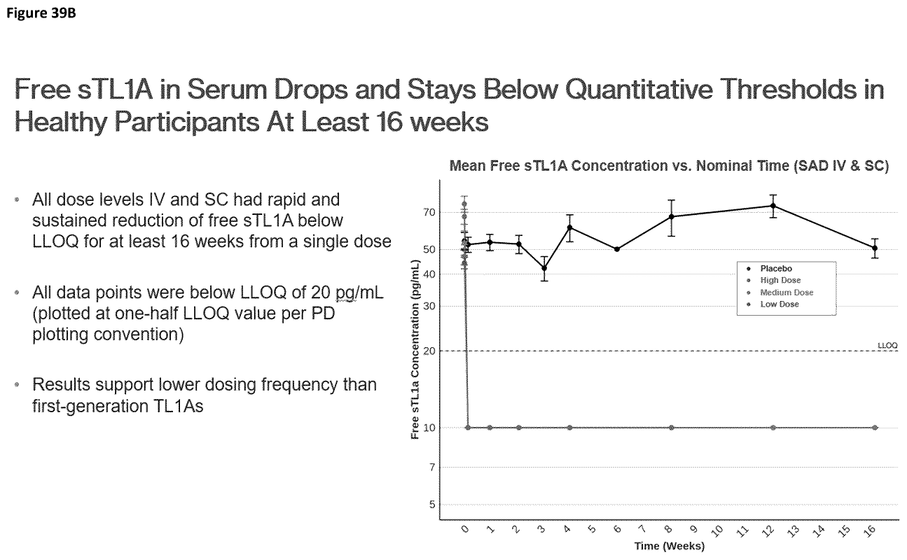

Additional results from a human pharmacodynamic study with three different doses of XmAb942 administered as a single dose IV or SC is shown in FIGS. 39A and B. As shown in FIG. 39A, dose-dependent and durable increases in total TL1A was observed at least 16 weeks after a single dose of XmAb942, consistent with high binding potency and extended serum exposure. As shown in FIG. 39B, all dose levels of VI and SC administered XmAb942 had rapid and sustained reduction of free sTL1A below LLOQ for at least 16 weeks from a single dose. All data points were below LLOQ of 20 pg/ml. Taken altogether, these results support lower dosing frequency than first-generation TL1As.

### Example 6: Clinical Studies

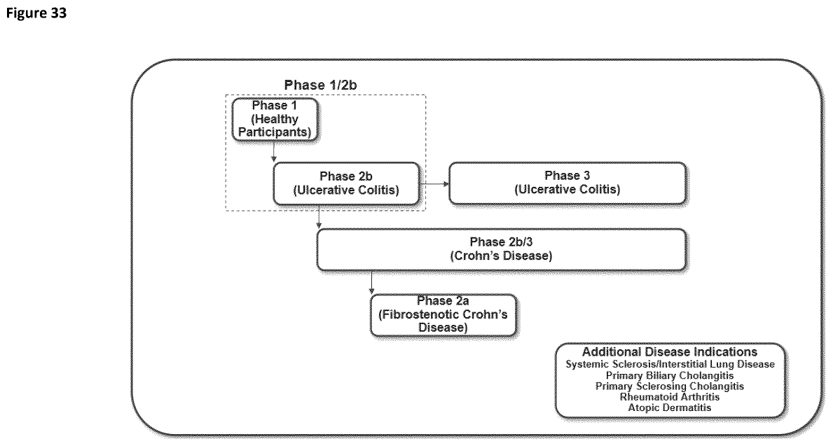

Human clinical studies to assess XmAb942 for the treatment of ulcerative colitis, Crohn's disease, Fibrostenotic Crohn's disease, and other additional disease indications will be carried out as outlined in FIG. 33. Details of a human phase 2b study in ulcerative colitis is shown in FIG. 36.

### EMBODIMENTS

1. A means for binding an antigen comprising means for binding human TL1a and means for increasing serum half-life of the means for binding the antigen.
2. A means for binding an antigen comprising means for binding human TL1a and means for reducing/removing binding to a cell surface receptor.
3. A pharmaceutical composition comprising:
   (a) means for binding an antigen comprising means for binding human TL1a and means for increasing serum half-life of the means for binding the antigen; and
   (b) a pharmaceutically acceptable carrier.
4. A pharmaceutical composition comprising:
   (a) means for binding human TL1a; and
   (b) a pharmaceutically acceptable carrier.
5. The pharmaceutical composition of embodiment 4 further comprising means for increasing serum half-life of the means for binding human TL1a.
6. A pharmaceutical composition comprising:
   (a) means for binding an antigen comprising means for binding human TL1a and means for reducing or reducing/removing binding to a cell surface receptor; and
   (b) a pharmaceutically acceptable carrier.
7. A method of inhibiting TL1A-mediated cell apoptosis in a subject in need thereof, wherein the method comprises administering to the subject the means for binding an antigen according to embodiments 1-2 or the pharmaceutical composition according to embodiments 3-6.
8. A method of inhibiting TL1A-mediated NFkB signaling in a subject in need thereof, wherein the method comprises administering to the subject the means for binding an antigen according to embodiments 1-2 or the pharmaceutical composition according to embodiments 3-6.
9. A method of inhibiting TL1A-induced cytokine secretion in a subject in need thereof, wherein the method comprises administering to the subject the means for binding an antigen according to embodiments 1-2 or the pharmaceutical composition according to embodiments 3-6.
10. The method of embodiment 9, wherein the cytokine is selected the following interleukin-1β (IL-1β); tumor necrosis factor-α (TNF-α); transforming growth factor-β (TGF-β); granulocyte-macrophage colony stimulating factor (GM-CSF); and interferon-γ (IFN-γ).
11. The method of any one of embodiments 7-10, wherein the subject has a TL1A-associated disease.
12. The method of embodiment 11, wherein the TL1A-associated disease is an inflammatory bowel disease.
13. The method of embodiment 11, wherein the TL1A-associated diseases is one of the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis.
14. A method for treating a TL1A-associated disease, wherein the method comprises administering to the subject in need thereof the means for binding an antigen according to embodiments 1-2 or the pharmaceutical composition according to embodiments 3-6.
15. The method of embodiment 13, wherein the TL1A-associated disease is an inflammatory bowel disease.
16. The method of a embodiment 13, wherein the TL1A-associated diseases is one of the following: ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis.
17. The method ofany one of embodiments 7-16, wherein the subject is a human.

Table 3 below links the above listed “means ” clauses to the relevant portions of the specifications.

| | Exemplified structures and/or materials |
|---|---|
| | and/or acts disclosed in the Specification |
| Recited function | of the application filed herewith |
| means for binding an antigen | linked to FIG. 11A, paragraph [0040], |
| comprising means for binding | “>XENP47942 |
| human TL1a and means for | 70416_041[TL1a]_H1L1_Fab |
| increasing serum half-life | IgG1_PVA_/S267K/M428L/N434S;” |
| of the means for binding | paragraph [00167], Example 1], |
| the antigen | FIG. 9A, “70416_041[TL1a]_H1L1,” |
| | FIG. 7, [00130], [0040], [0036] |
| means for binding human | linked to FIG. 9A, |
| TL1a; | “70416_041[TL1a]_HIL1.” |
| means for reducing/removing | linked to FIG. 2, [00133], [0040] |
| binding to a cell surface | |
| receptor | |
| means for increasing serum | linked to FIG. 7, [00130], [0040], |
| half-life of the means for | [0036] |
| binding the antigen | |

1.-66. (canceled)

67. An antibody comprising a TNF-like ligand 1A (TL1A) antigen binding domain, the TL1A binding domain comprising a variable light domain and a variable heavy domain selected from the following sets of variable light domains and variable heavy domains:
a) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
b) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
c) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
d) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
e) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
f) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
g) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
h) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
i) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
j) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80.

68. The antibody of claim 67, wherein the variable heavy domain and the variable light domain are selected from the following sets of variable heavy domains and variable light domains:
a) a variable heavy domain having an amino acid sequence that is at least 90% identical to SEQ ID NO:1, and a variable light domain having an amino acid sequence that is at least 90% identical to SEQ ID NO:5;
b) a variable heavy domain having an amino acid sequence that is at least 90% identical to SEQ ID NO:9, and a variable light domain having an amino acid sequence that is at least 90% identical to SEQ ID NO:13;
c) a variable heavy domain having an amino acid sequence that is at least 90% identical to SEQ ID NO:17, and a variable light domain having an amino acid sequence that is at least 90% identical to SEQ ID NO:21;
d) a variable heavy domain having an amino acid sequence that is at least 90% identical to SEQ ID NO:25, and a variable light domain having an amino acid sequence that is at least 90% identical to SEQ ID NO:29;
e) a variable heavy domain having an amino acid sequence that is at least 90% identical to SEQ ID NO:33, and a variable light domain having an amino acid sequence that is at least 90% identical to SEQ ID NO:37;
f) a variable heavy domain having an amino acid sequence that is at least 90% identical to SEQ ID NO:41, and a variable light domain having an amino acid sequence that is at least 90% identical to SEQ ID NO:45;
g) a variable heavy domain having an amino acid sequence that is at least 90% identical to SEQ ID NO:49, and a variable light domain having an amino acid sequence that is at least 90% identical to SEQ ID NO:53;
h) a variable heavy domain having an amino acid sequence that is at least 90% identical to SEQ ID NO:57 and a variable light domain having an amino acid sequence that is at least 90% identical to SEQ ID NO:61;
i) a variable heavy domain having an amino acid sequence that is at least 90% identical to SEQ ID NO:65, and a variable light domain having an amino acid sequence that is at least 90% identical to SEQ ID NO:69; and
j) a variable heavy domain having an amino acid sequence that is at least 90% identical to SEQ ID NO:73, and a variable light domain having an amino acid sequence that is at least 90% identical to SEQ ID NO:77.

69. The antibody of claim 67, wherein the variable heavy domain and the variable light domain are selected from the following sets of variable heavy domains and variable light domains:
a) a variable heavy domain having an amino acid sequence of SEQ ID NO:1, and a variable light domain having an amino acid sequence of SEQ ID NO:5;
b) a variable heavy domain having an amino acid sequence of SEQ ID NO:9, and a variable light domain having an amino acid sequence of SEQ ID NO:13;
c) a variable heavy domain having an amino acid sequence of SEQ ID NO: 17, and a variable light domain having an amino acid sequence of SEQ ID NO:21;
d) a variable heavy domain having an amino acid sequence of SEQ ID NO:25, and a variable light domain having an amino acid sequence of SEQ ID NO:29;
e) a variable heavy domain having an amino acid sequence of SEQ ID NO:33, and a variable light domain having an amino acid sequence of SEQ ID NO:37;
f) a variable heavy domain having an amino acid sequence of SEQ ID NO:41, and a variable light domain having an amino acid sequence of SEQ ID NO:45;
g) a variable heavy domain having an amino acid sequence of SEQ ID NO 49, and a variable light domain having an amino acid sequence of SEQ ID NO: 53;
h) a variable heavy domain having an amino acid sequence of SEQ ID NO:57 and a variable light domain having an amino acid sequence of SEQ ID NO:61;
i) a variable heavy domain having an amino acid sequence of SEQ ID NO:65, and a variable light domain having an amino acid sequence of SEQ ID NO:69; and
j) a variable heavy domain having an amino acid sequence of SEQ ID NO:73, and a variable light domain having an amino acid sequence of SEQ ID NO:77.

70. An anti-TL1A antibody comprising a heavy chain and a light chain; wherein the heavy chain and light chain selected from the following:
a) a heavy chain having an amino acid sequence of SEQ ID NO:123, and a light chain having an amino acid sequence of SEQ ID NO:124;
b) a heavy chain having an amino acid sequence of SEQ ID NO:117, and a light chain having an amino acid sequence of SEQ ID NO:118;
c) a heavy chain having an amino acid sequence of SEQ ID NO:119, and a light chain having an amino acid sequence of SEQ ID NO:120;
d) a heavy chain having an amino acid sequence of SEQ ID NO:121, and a light chain having an amino acid sequence of SEQ ID NO:122; and
e) a heavy chain having an amino acid sequence of SEQ ID NO:125, and a light chain having an amino acid sequence of SEQ ID NO:126.

71. A nucleic acid composition encoding an anti-TL1A antibody, wherein the composition comprises:
a) a first nucleic acid encoding a variable heavy domain and a second nucleic acid encoding a variable light domain, wherein the variable heavy domain and variable light domain are selected from the following:
i) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
ii) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
iii) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
iv) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
v) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
vi) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
vii) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
viii) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
ix) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
x) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80; or
b) a first nucleic acid encoding a heavy chain having an amino acid sequence of SEQ ID NO: 123, and a light chain having an amino acid sequence of SEQ ID NO: 124; or
c) a first nucleic acid encoding a heavy chain having an amino acid sequence of SEQ ID NO: 117, and a light chain having an amino acid sequence of SEQ ID NO: 118; or
d) a first nucleic acid encoding a heavy chain having an amino acid sequence of SEQ ID NO: 119, and a light chain having an amino acid sequence of SEQ ID NO: 120; or
e) a first nucleic acid encoding a heavy chain having an amino acid sequence of SEQ ID NO: 121, and a light chain having an amino acid sequence of SEQ ID NO: 122; or
f) a first nucleic acid encoding a heavy chain having an amino acid sequence of SEQ ID NO: 125, and a light chain having an amino acid sequence of SEQ ID NO: 126.

72. An expression vector composition comprising the first nucleic acid of claim 71, and the second nucleic acid of claim 71.

73. A host cell comprising the expression vector composition of claim 72.

74. A method of making an anti-TL1A antibody comprising culturing a host cell comprising a nucleic acid composition under conditions wherein the anti-TL1A antibody is expressed and recovering the anti-TL1A antibody, wherein the nucleic acid composition comprises:
a) a first nucleic acid encoding a variable heavy domain and a second nucleic acid encoding a variable light domain, and wherein the variable heavy domain and variable light domain are selected from the following:
i) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
ii) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
iii) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
iv) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
v) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
vi) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
vii) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
viii) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
ix) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
x) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80; or
b) a first nucleic acid encoding a heavy chain having an amino acid sequence of SEQ ID NO: 123, and a light chain having an amino acid sequence of SEQ ID NO: 124;
c) a first nucleic acid encoding a heavy chain having an amino acid sequence of SEQ ID NO: 117, and a light chain having an amino acid sequence of SEQ ID NO: 118; or
d) a first nucleic acid encoding a heavy chain having an amino acid sequence of SEQ ID NO: 119. and a light chain having an amino acid sequence of SEQ ID NO: 120; or
e) a first nucleic acid encoding a heavy chain having an amino acid sequence of SEQ ID NO: 121, and a light chain having an amino acid sequence of SEQ ID NO: 122; or
f) a first nucleic acid encoding a heavy chain having an amino acid sequence of SEQ ID NO: 125, and a light chain having an amino acid sequence of SEQ ID NO: 126.

75. A method of inhibiting or reducing TL1A-mediated cell apoptosis in a subject in need thereof, wherein the method comprises administrating to the subject an anti-TL1A antibody selected from the following:
a) an anti-TL1A antibody comprising a variable heavy domain and a variable light domain, wherein the variable heavy domain and variable light domain are selected from the following:
i) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
ii) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
iii) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
iv) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
v) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
vi) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
vii) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
viii) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
ix) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
x) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80; or
b) a an anti-TL1A antibody comprising a heavy chain having an amino acid sequence of SEQ ID NO: 123, and a light chain having an amino acid sequence of SEQ ID NO: 124; or
c) a an anti-TL1A antibody comprising a heavy chain having an amino acid sequence of SEQ ID NO: 117, and a light chain having an amino acid sequence of SEQ ID NO: 118; or
d) a an anti-TL1A antibody comprising a heavy chain having an amino acid sequence of SEQ ID NO: 119, and a light chain having an amino acid sequence of SEQ ID NO: 120; or
e) a an anti-TL1A antibody comprising a heavy chain having an amino acid sequence of SEQ ID NO: 121, and a light chain having an amino acid sequence of SEQ ID NO: 122; or
f) a an anti-TL1A antibody comprising a heavy chain having an amino acid sequence of SEQ ID NO: 125, and a light chain having an amino acid sequence of SEQ ID NO: 126.

76. A method of inhibiting or reducing TL1A-mediated NFkB signaling in a subject in need thereof, wherein the method comprises administrating to the subject an anti-TL1A antibody selected from the following:
a) an anti-TL1A antibody comprising a variable heavy domain and a variable light domain, wherein the variable heavy domain and variable light domain are selected from the following:
i) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
ii) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
iii) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
iv) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
v) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
vi) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
vii) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
viii) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
ix) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
x) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80; or
b) a an anti-TL1A antibody comprising a heavy chain having an amino acid sequence of SEQ ID NO: 123, and a light chain having an amino acid sequence of SEQ ID NO: 124; or
c) a an anti-TL1A antibody comprising a heavy chain having an amino acid sequence of SEQ ID NO: 117, and a light chain having an amino acid sequence of SEQ ID NO: 118; or
d) a an anti-TL1A antibody comprising a heavy chain having an amino acid sequence of SEQ ID NO: 119, and a light chain having an amino acid sequence of SEQ ID NO: 120; or
e) a an anti-TL1A antibody comprising a heavy chain having an amino acid sequence of SEQ ID NO: 121, and a light chain having an amino acid sequence of SEQ ID NO: 122; or
f) a an anti-TL1A antibody comprising a heavy chain having an amino acid sequence of SEQ ID NO: 125, and a light chain having an amino acid sequence of SEQ ID NO: 126.

77. A method of inhibiting or reducing TL1A-induced cytokine secretion in a subject in need thereof, wherein the method comprises administrating to the subject an anti-TL1A antibody selected from the following:
a) an anti-TL1A antibody comprising a variable heavy domain and a variable light domain, wherein the variable heavy domain and variable light domain are selected from the following:
i) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
ii) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
iii) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
iv) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
v) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
vi) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
vii) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
viii) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
ix) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
x) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80; or
b) a an anti-TL1A antibody comprising a heavy chain having an amino acid sequence of SEQ ID NO: 123, and a light chain having an amino acid sequence of SEQ ID NO: 124; or
c) a an anti-TL1A antibody comprising a heavy chain having an amino acid sequence of SEQ ID NO: 117, and a light chain having an amino acid sequence of SEQ ID NO: 118; or
d) a an anti-TL1A antibody comprising a heavy chain having an amino acid sequence of SEQ ID NO: 119, and a light chain having an amino acid sequence of SEQ ID NO: 120; or
e) a an anti-TL1A antibody comprising a heavy chain having an amino acid sequence of SEQ ID NO: 121, and a light chain having an amino acid sequence of SEQ ID NO: 122; or
F) a an anti-TL1A antibody comprising a heavy chain having an amino acid sequence of SEQ ID NO: 125, and a light chain having an amino acid sequence of SEQ ID NO: 126.

78. The method of claim 77, wherein the cytokine is selected from the following cytokines: interferon-γ (IFN-γ), interleukin-1β (IL-1β); tumor necrosis factor-α (TNF-α); transforming growth factor-β (TGF-β); and granulocyte-macrophage colony stimulating factor (GM-CSF).

79. A method for treating a TL1A-associated disease, wherein the method comprises administrating to the subject an anti-TL1A antibody selected from the following:
a) an anti-TL1A antibody comprising a variable heavy domain and a variable light domain, wherein the variable heavy domain and variable light domain are selected from the following:
i) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
ii) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
iii) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
iv) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
v) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
vi) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
vii) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
viii) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
ix) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
x) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80; or
b) a an anti-TL1A antibody comprising a heavy chain having an amino acid sequence of SEQ ID NO: 123, and a light chain having an amino acid sequence of SEQ ID NO: 124; or
c) a an anti-TL1A antibody comprising a heavy chain having an amino acid sequence of SEQ ID NO: 117, and a light chain having an amino acid sequence of SEQ ID NO: 118; or
d) a an anti-TL1A antibody comprising a heavy chain having an amino acid sequence of SEQ ID NO: 119, and a light chain having an amino acid sequence of SEQ ID NO: 120; or
e) a an anti-TL1A antibody comprising a heavy chain having an amino acid sequence of SEQ ID NO: 121, and a light chain having an amino acid sequence of SEQ ID NO: 122; or
f) a an anti-TL1A antibody comprising a heavy chain having an amino acid sequence of SEQ ID NO: 125, and a light chain having an amino acid sequence of SEQ ID NO: 126.

80. The method of claim 79, wherein the TL1A-associated disease is: an inflammatory bowel disease, ulcerative colitis, Crohn's Disease, fibrostenotic Crohn's Disease, systemic sclerosis, interstitial lung disease, primary biliary cholangitis, primary sclerosing cholangitis, rheumatoid arthritis, and atopic dermatitis.

81. A TNF-like ligand 1A (TL1A) antigen binding domain, the TL1A binding domain comprising a variable light domain and a variable heavy domain selected from the following sets of variable light domains and variable heavy domains:
a) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
b) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
c) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
d) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
e) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
f) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
g) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
h) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
i) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
j) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80.

82. The TL1A antigen binding domain of claim 81, wherein the variable heavy domain and the variable light domain are selected from the following sets of variable heavy domains and variable light domains:
a) a variable heavy domain having an amino acid sequence that is at least 90% identical to SEQ ID NO:1, and a variable light domain having an amino acid sequence that is at least 90% identical to SEQ ID NO:5;
b) a variable heavy domain having an amino acid sequence that is at least 90% identical to SEQ ID NO:9, and a variable light domain having an amino acid sequence that is at least 90% identical to SEQ ID NO:13;
c) a variable heavy domain having an amino acid sequence that is at least 90% identical to SEQ ID NO:17, and a variable light domain having an amino acid sequence that is at least 90% identical to SEQ ID NO:21;
d) a variable heavy domain having an amino acid sequence that is at least 90% identical to SEQ ID NO:25, and a variable light domain having an amino acid sequence that is at least 90% identical to SEQ ID NO:29;
e) a variable heavy domain having an amino acid sequence that is at least 90% identical to SEQ ID NO:33, and a variable light domain having an amino acid sequence that is at least 90% identical to SEQ ID NO:37;
f) a variable heavy domain having an amino acid sequence that is at least 90% identical to SEQ ID NO:41, and a variable light domain having an amino acid sequence that is at least 90% identical to SEQ ID NO:45;
g) a variable heavy domain having an amino acid sequence that is at least 90% identical to SEQ ID NO:49, and a variable light domain having an amino acid sequence that is at least 90% identical to SEQ ID NO:53;
h) a variable heavy domain having an amino acid sequence that is at least 90% identical to SEQ ID NO:57, and a variable light domain having an amino acid sequence that is at least 90% identical to SEQ ID NO:61;
i) a variable heavy domain having an amino acid sequence that is at least 90% identical to SEQ ID NO:65, and a variable light domain having an amino acid sequence that is at least 90% identical to SEQ ID NO:69; and
j) a variable heavy domain having an amino acid sequence that is at least 90% identical to SEQ ID NO:73, and a variable light domain having an amino acid sequence that is at least 90% identical to SEQ ID NO:77.

83. The TL1A antigen binding domain of claim 81, wherein the variable heavy domain and the variable light domain are selected from the following sets of variable heavy domains and variable light domains:
a) a variable heavy domain having an amino acid sequence of SEQ ID NO:1, and a variable light domain having an amino acid sequence of SEQ ID NO:5;
b) a variable heavy domain having an amino acid sequence of SEQ ID NO:9, and a variable light domain having an amino acid sequence of SEQ ID NO:13;
c) a variable heavy domain having an amino acid sequence of SEQ ID NO: 17, and a variable light domain having an amino acid sequence of SEQ ID NO:21;
d) a variable heavy domain having an amino acid sequence of SEQ ID NO:25, and a variable light domain having an amino acid sequence of SEQ ID NO:29;
e) a variable heavy domain having an amino acid sequence of SEQ ID NO:33, and a variable light domain having an amino acid sequence of SEQ ID NO:37;
f) a variable heavy domain having an amino acid sequence of SEQ ID NO:41, and a variable light domain having an amino acid sequence of SEQ ID NO:45;
g) a variable heavy domain having an amino acid sequence of SEQ ID NO 49, and a variable light domain having an amino acid sequence of SEQ ID NO: 53;
h) a variable heavy domain having an amino acid sequence of SEQ ID NO: 57 and a variable light domain having an amino acid sequence of SEQ ID NO:61;
i) a variable heavy domain having an amino acid sequence of SEQ ID NO:65, and a variable light domain having an amino acid sequence of SEQ ID NO:69; and
j) a variable heavy domain having an amino acid sequence of SEQ ID NO:73, and a variable light domain having an amino acid sequence of SEQ ID NO:77.

84. A nucleic acid composition encoding the TL1A antigen binding domain, wherein the composition comprises a first nucleic acid encoding a variable heavy domain and a second nucleic acid encoding a variable light domain, wherein the variable heavy domain and variable light domain are selected from the following:
i) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
ii) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
iii) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
iv) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
v) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
vi) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
vii) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
viii) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
ix) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
x) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80.

85. An expression vector composition comprising:
a) a first expression vector comprising the first nucleic acid of claim 84; and
b) a second expression vector comprising the second nucleic acid of claim 84.

86. A host cell comprising the expression vector composition of claim 85.

87. A method of making a TL1A binding domain comprising culturing a host cell comprising a nucleic acid composition under conditions wherein the TL1A binding domain is expressed and recovering the TL1A binding domain,
wherein the nucleic acid composition comprises a first nucleic acid encoding a variable heavy domain and a second nucleic acid encoding a variable light domain, and wherein the variable heavy domain and variable light domain are selected from the following:
i) a variable heavy domain comprising a vhCDR1 having an amino acid sequence of SEQ ID NO:2, a vhCDR2 having an amino acid sequence of SEQ ID NO:3, and a vhCDR3 having an amino acid sequence of SEQ ID NO:4, and a variable light domain comprising a vlCDR1 having an amino acid sequence of SEQ ID NO:6, a vlCDR2 having an amino acid sequence of SEQ ID NO:7, and a vlCDR3 having an amino acid sequence of SEQ ID NO:8;
ii) a variable heavy domain comprising a vhCDR1 having an amino acid sequence of SEQ ID NO:10, a vhCDR2 having an amino acid sequence of SEQ ID NO:11, and a vhCDR3 having an amino acid sequence of SEQ ID NO:12, and a variable light domain comprising a vlCDR1 having an amino acid sequence of SEQ ID NO:14, a vlCDR2 having an amino acid sequence of SEQ ID NO:15, and a vlCDR3 having an amino acid sequence of SEQ ID NO:16;
iii) a variable heavy domain comprising a vhCDR1 having an amino acid sequence of SEQ ID NO:18, a vhCDR2 having an amino acid sequence of SEQ ID NO:19, and a vhCDR3 having an amino acid sequence of SEQ ID NO:20, and a variable light domain comprising a vlCDR1 having an amino acid sequence of SEQ ID NO:22, a vlCDR2 having an amino acid sequence of SEQ ID NO:23, and a vlCDR3 having an amino acid sequence of SEQ ID NO:24;
iv) a variable heavy domain comprising a vhCDR1 having an amino acid sequence of SEQ ID NO:26, a vhCDR2 having an amino acid sequence of SEQ ID NO:27, and a vhCDR3 having an amino acid sequence of SEQ ID NO:28, and a variable light domain comprising a vlCDR1 having an amino acid sequence of SEQ ID NO:30, a vlCDR2 having an amino acid sequence of SEQ ID NO:31, and a vlCDR3 having an amino acid sequence of SEQ ID NO:32;
v) a variable heavy domain comprising a vhCDR1 having an amino acid sequence of SEQ ID NO:34, a vhCDR2 having an amino acid sequence of SEQ ID NO:35, and a vhCDR3 having an amino acid sequence of SEQ ID NO:36, and a variable light domain comprising a vlCDR1 having an amino acid sequence of SEQ ID NO:38, a vlCDR2 having an amino acid sequence of SEQ ID NO:39, and a vlCDR3 having an amino acid sequence of SEQ ID NO:40;
vi) a variable heavy domain comprising a vhCDR1 having an amino acid sequence of SEQ ID NO:42, a vhCDR2 having an amino acid sequence of SEQ ID NO:43, and a vhCDR3 having an amino acid sequence of SEQ ID NO:44, and a variable light domain comprising a vlCDR1 having an amino acid sequence of SEQ ID NO:46, a vlCDR2 having an amino acid sequence of SEQ ID NO:47, and a vlCDR3 having an amino acid sequence of SEQ ID NO:48;
vii) a variable heavy domain comprising a vhCDR1 having an amino acid sequence of SEQ ID NO:50, a vhCDR2 having an amino acid sequence of SEQ ID NO:51, and a vhCDR3 having an amino acid sequence of SEQ ID NO:52, and a variable light domain comprising a vlCDR1 having an amino acid sequence of SEQ ID NO:54, a vlCDR2 having an amino acid sequence of SEQ ID NO:55, and a vlCDR3 having an amino acid sequence of SEQ ID NO:56;
viii) a variable heavy domain comprising a vhCDR1 having an amino acid sequence of SEQ ID NO:58, a vhCDR2 having an amino acid sequence of SEQ ID NO:59, and a vhCDR3 having an amino acid sequence of SEQ ID NO:60, and a variable light domain comprising a vlCDR1 having an amino acid sequence of SEQ ID NO:62, a vlCDR2 having an amino acid sequence of SEQ ID NO:63, and a vlCDR3 having an amino acid sequence of SEQ ID NO:64;
ix) a variable heavy domain comprising a vhCDR1 having an amino acid sequence of SEQ ID NO:66, a vhCDR2 having an amino acid sequence of SEQ ID NO:67, and a vhCDR3 having an amino acid sequence of SEQ ID NO:68, and a variable light domain comprising a vlCDR1 having an amino acid sequence of SEQ ID NO:70, a vlCDR2 having an amino acid sequence of SEQ ID NO:71, and a vlCDR3 having an amino acid sequence of SEQ ID NO:72; and
x) a variable heavy domain comprising a vhCDR1 having an amino acid sequence of SEQ ID NO:74, a vhCDR2 having an amino acid sequence of SEQ ID NO:75, and a vhCDR3 having an amino acid sequence of SEQ ID NO:76, and a variable light domain comprising a vlCDR1 having an amino acid sequence of SEQ ID NO:78, a vlCDR2 having an amino acid sequence of SEQ ID NO:79, and a vlCDR3 having an amino acid sequence of SEQ ID NO:80.

88. A pharmaceutical composition comprising:
a) a means for binding human TL1A; and
b) a pharmaceutically acceptable carrier.
